(19) United States
(12) Patent Application Publication
(10) Pub. No.: US 2024/0115569 A1
(43) Pub. Date: Apr. 11, 2024

(54) METHODS FOR BLOCKING HER2 SIGNALING FOR TREATING PULMONARY FIBROSIS

(71) Applicant: CEDARS-SINAI MEDICAL CENTER, Los Angeles, CA (US)

(72) Inventors: Paul W. Noble, Beverly Hills, CA (US); Dianhua Jiang, Encino, CA (US)

(73) Assignee: CEDARS-SINAI MEDICAL CENTER, Los Angeles, CA (US)

(21) Appl. No.: 18/276,115

(22) PCT Filed: Feb. 11, 2022
(86) PCT No.: PCT/US2022/016213

§ 371 Date: Aug. 7, 2023

Related U.S. Application Data
(60) Provisional application No. 63/149,102, filed on Feb. 12, 2021.

(51) Int. Cl. A61K 31/517; A61K 47/68; A61P 11/00

(52) U.S. Cl. CPC A61K 31/517; A61K 47/6803; A61P 11/00

(57) ABSTRACT

The invention of the present disclosure relates to methods for diagnosing and for treating a progressive lung disease in a subject. In various embodiments, the method for treating a progressive lung disease in a subject includes administering a pharmaceutical composition comprising a Human Epidermal Growth Factor Receptor 2 (HER2) blocking agent and a pharmaceutically acceptable carrier to the subject, wherein the method improves clinical outcome compared to an untreated control.

### CROSS-REFERENCE TO RELATED APPLICATIONS

This application includes a claim of priority under 35 U.S.C. § 119(e) to U.S. provisional patent application No. 63/149,102, filed Feb. 12, 2021, the entirety of which is hereby incorporated by reference.

### STATEMENT REGARDING FEDERALLY SPONSORED RESEARCH OR DEVELOPMENT

This invention was made with government support under Grant Nos. HL108793 and HL150829 awarded by National Institutes of Health. The Government has certain rights in the invention.

### FIELD OF THE DISCLOSURE

The present disclosure relates to methods for treating a progressive tissue fibrosis.

### BACKGROUND OF THE INVENTION

Progressive tissue fibrosis is a major cause of morbidity and mortality, caused by dysregulated wound healing response to tissue injury. Idiopathic pulmonary fibrosis (IPF) is a chronic and progressive lung disease of unknown cause that leads to the destruction of the gas-exchanging regions of the lung with accumulation of fibroblast that produce massive extracellular matrix (ECM). Although pirfenidone and nintedanib have been approved by the FDA to slow IPF progression and both therapies have been shown to reduce the rate of decline in the forced vital capacity, neither of these agents improves lung function or reduces fibrosis. The next breakthrough needs to identify novel targets to improve IPF treatment. Additional therapies are needed.

Lung fibroblasts reside in the interstitial spaces between the alveolar and capillary basal laminae under normal conditions. Pathologic hallmarks of IPF are emergence of fibroblastic foci and destruction of basement membrane. Lung fibroblasts are heterogeneous cells and histological observation of the fibroblastic foci indicates that a population of fibroblasts may migrate or invade through alveolar basement membranes after lung injury. It has been shown that the invasive phenotype of lung fibroblasts promotes severe fibrosis.

### SUMMARY OF THE INVENTION

The present disclosure provides a method for treating a subject with a progressive lung disease. In various embodiments, the method includes administering a pharmaceutical composition including a Human Epidermal Growth Factor Receptor 2 (HER2) blocking agent and a pharmaceutically acceptable carrier to the subject, the method improving clinical outcome compared to an untreated control. According to some embodiments, the progressive lung disease is pulmonary fibrosis. According to some embodiments, the progressive lung disease is idiopathic pulmonary fibrosis (IPF). According to some embodiments, the HER2 blocking agent includes an anti-HER2 antibody, a small molecule HER2 inhibitor, or both. According to some embodiments, the anti-HER2 antibody includes one or more of trastuzumab, trastuzumab-pkrb, trastuzumab-anns, trastuzumab-dkst, trastuzumab-dttb, trastuzumab-qyyp, margutuximab-cmkb, or pertuzumab. According to some embodiments, the anti-HER2 antibody includes trastuzumab and pertuzumab. According to some embodiments, the anti-HER2 antibody includes a recombinant hyaluronidase. According to some embodiments, the anti-HER2 antibody includes an antibody drug conjugate. According to some embodiments, the antibody-drug conjugate includes trastuzumab entansine, trastuzumab deruxtecan (fam-trastuzumab deruxtecan-nxki); or disitamab vedotin. According to some embodiments, the small molecule HER2 inhibitor is one or more of lapatinib, neratinib, tucatanib, pyrotinib, afatinib dimaleate, dacomitinib, or a pharmaceutically acceptable salt thereof. According to some embodiments, the anti-HER2 antibody includes one or more of trastuzumab, trastuzumab-pkrb, trastuzumab-anns, trastuzumab-dkst, trastuzumab-dttb, trastuzumab-qyyp, margutuximab-cmkb, or pertuzumab; and the small molecule HER2 inhibitor is one or more of lapatinib, neratinib, tucatanib, pyrotinib, afatinib dimaleate, dacomitinib, ribociclib, palbociclib, abemaciclib, or a pharmaceutically acceptable salt thereof. According to some embodiments, the clinical outcome is progression free survival or overall survival. According to some embodiments, the subject is treated when one or more, two or more, three or more, four or more, or all of CD108 (SEMA7A), CD142 (F3), CD49F (ITGA6), PD-L1, and PD-L2 encoded by RNA from the subject is upregulated compared to RNA encoded by a normal healthy control subject. According to some embodiments, CD108 (SEMA7A) encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; CD142 (F3) encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; CD49F (ITGA6) encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; PD-L1 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; and/or PD-L2 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject. According to some embodiments, the subject is treated when one or more, two or more, three or more, four or more, five or more, or all of transcription factors FOXF1, CREBRF, TSC22D1, MXI1, NFE2L2, and KLF9 encoded by RNA from the subject are downregulated compared to RNA encoded by a normal healthy control subject. According to some embodiments, the transcription factor FOXF1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; the transcription factor CREBRF encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; the transcription factor TSC22D1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; the transcription factor MXI1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; the transcription factor NFE2L2 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; and/or the transcription factor KLF9 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject. According to some embodiments, the subject is treated when transcription factors HMGA2 and/or DPF3 encoded by RNA from the subject are upregulated compared to RNA encoded by a normal healthy control subject. According to some embodiments, the transcription factor HMGA2 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject; and/or the transcription factor DPF3 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA encoded by the normal healthy control subject. According to some embodiments, the subject is treated when regulation of lncRNA from the subject, which comprises one or more of LINC00152 and FENDRR, is different compared to regulation of lncRNA from a normal healthy control subject such that: the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject; and/or the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject.

In various embodiments, the subject does not have cancer. In various embodiments, the method further includes identifying the subject in need of pulmonary fibrosis treatment before administering the pharmaceutical composition including the HER2 blocking agent.

According to various embodiments, the method decreases one or more symptoms of the fibrotic lung disease; or the method increases repair of a lung injury; or the method slows progression of the fibrotic lung disease; or the method decreases migration, invasion or both by lung fibroblasts obtained from the subject compared to an untreated control; or the method increases renewal of alveolar epithelial cell 2 (AEC2) cells; or a combination thereof.

The present disclosure provides a method for identifying a subject with a progressive lung disease as one who can benefit therapeutically from being treated with a pharmaceutical composition comprising a Human Epidermal Growth Factor Receptor 2 (HER2) blocking agent. In various embodiments, the method includes (1) obtaining a biological sample from the subject, wherein the biological sample comprises non-invasive lung fibroblasts, invasive lung fibroblasts, or both non-invasive lung fibroblasts and invasive lung fibroblasts; and (2) determining an RNA profile for the biological sample by: (i) identifying and quantifying expression of RNA from the lung fibroblasts in the biological sample by RNA sequencing; and (ii) comparing the RNA profile for the lung fibroblasts in the biological sample to an RNA profile of lung fibroblasts obtained from a normal healthy control subject. According to some embodiments, the subject is identified as the one who can benefit therapeutically from being treated with the pharmaceutical composition comprising the HER2 blocking agent, when the RNA profile from the biological sample is distinguishable from the RNA profile from the healthy control subject.

According to some embodiments, a method for identifying a subject with a progressive lung disease as one who can benefit therapeutically from being treated with a pharmaceutical composition comprising a HER2 blocking agent includes (3) determining an index of migration for the lung fibroblasts in the biological sample from the subject by: (i) in a 3D in vitro migration assay, quantifying the lung fibroblasts that have migrated through a porous membrane and a matrix comprising extracellular matrix proteins towards a fibroblast chemoattractant after about 24 hours; and (ii) normalizing a count of the lung fibroblasts in (3)(i) to a number of lung fibroblasts obtained from a control subject that have migrated through the membrane and matrix obtained from a control subject, thereby determining the index of migration. According to some embodiments, a method for identifying a subject with a progressive lung disease as one who can benefit therapeutically from being treated with a pharmaceutical composition comprising a HER2 blocking agent includes (4) determining an index of invasion for the lung fibroblasts derived from the biological sample from the subject by: (i) in a 3D in vitro invasion assay, quantifying the lung fibroblasts derived from the biological sample from the subject that have migrated through a porous membrane and into a matrigel matrix towards a fibroblast chemoattractant after about 24 hours; and (ii) normalizing a count of the lung fibroblasts in (4)(i) to a number of lung fibroblasts obtained from a control subject that have migrated through a porous membrane and into a matrigel matrix towards the fibroblast chemoattractant, thereby determining the index of invasion. According to some embodiments, the subject is identified as the one who can benefit therapeutically from being treated with the pharmaceutical composition comprising the HER2 blocking agent, when the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold higher than an index of migration of fibroblasts derived from a control. According to some embodiments, the subject is identified as the one who can benefit therapeutically from being treated with the pharmaceutical composition comprising the HER2 blocking agent, when the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold lower than an index of migration of fibroblasts derived from a control.

According to some embodiments, the subject is identified as the one who can benefit therapeutically from being treated with the pharmaceutical composition comprising the HER2 blocking agent, when: CD108 (SEMA7A) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to RNA from lung fibroblasts of the normal healthy control subject; CD142 (F3) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; CD49F (ITGA6) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; PD-L1 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or PD-L2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when: transcription factor FOXF1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor CREBRF encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor TSC22D1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor MXI1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor NFE2L2 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or transcription factor KLF9 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when: transcription factor HMGA2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or transcription factor DPF3 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when regulation of lncRNA from the subject, which comprises one or more of LINC00152 and FENDRR, is different compared to regulation of lncRNA from the normal healthy control subject such that: the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject; and/or the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject.

The present disclosure provides a method for treating a subject with a progressive lung disease and identified as one who can benefit therapeutically from being treated with a pharmaceutical composition comprising a Human Epidermal Growth Factor Receptor 2 (HER2) blocking agent. In various embodiments, the method includes treating the identified subject with the pharmaceutical composition comprising the HER2 blocking agent such that clinical outcome improves by the treating. According to some embodiments, the subject is identified as the one who can benefit therapeutically from being treated with the pharmaceutical composition by: (1) obtaining or having obtained a biological sample from the subject, wherein the biological sample comprises non-invasive lung fibroblasts, invasive lung fibroblasts, or both non-invasive lung fibroblasts and invasive lung fibroblasts; and (2) determining or having determined an RNA profile for the biological sample by: (i) identifying and quantifying or having identified and quantified expression of RNA from the lung fibroblasts in the biological sample by RNA sequencing; and (ii) comparing or having compared the RNA profile for the lung fibroblasts in the biological sample to an RNA profile of lung fibroblasts obtained from a healthy control subject. According to some embodiments, the subject is identified as the one who can benefit therapeutically from being treated with the pharmaceutical composition comprising the HER2 blocking agent, when the RNA profile from the biological sample is distinguishable from the RNA profile from the healthy control subject.

According to some embodiments, the subject is identified as the one who can benefit therapeutically from being treated with the pharmaceutical composition by (3) determining or having determined an index of migration for the lung fibroblasts in the biological sample from the subject by: (i) in a 3D in vitro migration assay, quantifying or having quantified the lung fibroblasts that have migrated through a porous membrane and a matrix comprising extracellular matrix proteins towards a fibroblast chemoattractant after about 24 hours; and (ii) normalizing or having normalized a count of the lung fibroblasts in (3)(i) to a number of lung fibroblasts obtained from a control subject that have migrated through the membrane and matrix obtained from a control subject, thereby determining the index of migration. According to some embodiments, the subject is identified as the one who can benefit therapeutically from being treated with the pharmaceutical composition by (4) determining or having determined an index of invasion for the lung fibroblasts derived from the biological sample from the subject by: (i) in a 3D in vitro invasion assay, quantifying or having quantified the lung fibroblasts derived from the biological sample from the subject that have migrated through a porous membrane and into a matrigel matrix towards a fibroblast chemoattractant after about 24 hours; and (ii) normalizing or having normalized a count of the lung fibroblasts in (4)(i) to a number of lung fibroblasts obtained from a control subject that have migrated through a porous membrane and into a matrigel matrix towards the fibroblast chemoattractant, thereby determining the index of invasion. According to some embodiments, the subject is identified as the one who can benefit therapeutically from being treated with the pharmaceutical composition comprising the HER2 blocking agent, when the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold higher than an index of migration of fibroblasts derived from a control. According to some embodiments, the subject is identified as the one who can benefit therapeutically from being treated with the pharmaceutical composition comprising the HER2 blocking agent, when the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold lower than an index of migration of fibroblasts derived from a control.

According to some embodiments, the subject is identified as the one who can benefit therapeutically from being treated with the pharmaceutical composition comprising the HER2 blocking agent, when: CD108 (SEMA7A) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to RNA from lung fibroblasts of the normal healthy control subject; CD142 (F3) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; CD49F (ITGA6) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; PD-L1 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or PD-L2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when: transcription factor FOXF1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor CREBRF encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor TSC22D1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor MXI1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; transcription factor NFE2L2 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or transcription factor KLF9 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when: transcription factor HMGA2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or transcription factor DPF3 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when regulation of lncRNA from the subject, which comprises one or more of LINC00152 and FENDRR, is different compared to regulation of lncRNA from the normal healthy control subject such that: the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject; and/or the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject.

### BRIEF DESCRIPTION OF THE DRAWINGS

FIG. 1A-FIG. 1D show invasion assays on normal and IPF lung fibroblasts and quality control of single cell RNA-seq. of IPF-1. The cell invasion assay was performed on normal and IPF fibroblasts shown in FIG. 1A. Cell number was counted on the images taken after the assay. FIG. 1B depicts a graph of the number of invaded cells in counted on the images from FIG. 1A. Data are the mean±SEM, **P<0.01, ***P<0.001 by student's t test.

FIG. 1C depicts the distribution of number of total genes (nFeature_RNA) and total molecules (nCount_RNA) detected per cell before quality control. Cells with more than 1000 genes were selected for further analysis (blue dashed lines). Distribution of percentage of mitochondria genes (percent.mt) per cell before quality control. Cells with less than 15% mitochondrial UMIs were selected for further analysis (blue dashed lines). FIG. 1D depicts the distribution of number of total genes (nFeature_RNA) and total molecules (nCount_RNA) detected per cell after quality control. Cells with more than 1000 genes were selected for further analysis (blue dashed lines). Distribution of percentage of mitochondria genes (percent.mt) per cell after quality control.

FIG. 2A-FIG. 2D depict single cell RNA-seq and clustering of invasive and non-invasive lung fibroblasts. FIG. 2A shows t-SNE projection of 9,315 single non-invasive and invasive lung fibroblasts of IPF1. FIG. 2B shows four distinct clusters that were detected and assigned by different colors. FIG. 2C depicts a heatmap of all significant genes in the four clusters shown in FIG. 2B. FIG. 2D depicts violin plots showing signature gene expression across the four clusters in FIG. 2B. non-in, non-invasive, in, invasive.

FIG. 3A-FIG. 3D show visualization of fibrosis related genes. FIG. 3A depicts violin plots showing visualization of fibrosis related markers HAS2, SERPINE1, CD274, PDCDILG2, ILI1 and myofibroblasts related markers ACTA2, EN1 and CTGF (Wilcoxon, p<2.2e-16 each comparison). FIG. 3B shows visualization of fibrosis related markers HAS2, SERPINE1, and CD274, PDCDILG2 by UMAP. FIG. 3C shows visualization of fibrosis related marker IL11 and myofibroblasts related markers ACTA2, EN1 and CTGF by UMAP. FIG. 3D shows t-SNE plots of proliferative related markers, MKI67, CDK1, HMAIR and PCNA gene expression.

FIG. 4A-FIG. 4D show integration of invasive and non-invasive fibroblasts from 4 IPF patients and normal controls. Visualization of sample integration. FIG. 4A shows visualization of sample integration. FIG. 4B shows visualization of invasive and non-invasive fibroblast distribution. FIG. 4C shows sub-clusters of invasive and non-invasive fibroblasts. FIG. 4D shows visualization of invasive and non-invasive specific genes in integrated data.

FIG. 5A-FIG. 5E depict metagene analysis and clustering of invasive and non-invasive lung fibroblasts. FIG. 5A shows the metagene profile for each cluster of non-invasive and invasive lung fibroblasts. Arrow marks overexpressed and under-expressed metagene signatures. FIG. 5B depicts correlation spanning tree on metagene data. Non-in: non-invasive; in: invasive. FIG. 5C depicts correlation spanning tree on metagene data. Color code indicate pseudotime score of the cells. FIG. 5D depicts correlation spanning tree on K-nearest neighbor tree. Non-in: non-invasive; in: invasive. FIG. 5E depicts correlation spanning tree on K-nearest neighbor tree. Color code indicate pseudotime score of the cells. Non-in: non-invasive; in: invasive.

FIG. 6A-FIG. 6I show that cell surface markers were identified to distinguish invasive and non-invasive fibroblasts. FIG. 6A visualization of SEMA7A, F3 and ITGA6 using t-SNE plot in the invasive and non-invasive clusters. FIG. 6B depicts visualization of SEMA7A, F3 and ITGA6 using violin plots in the invasive and non-invasive clusters. FIG. 6C depicts cell surface expression of SEMA7A, F3 and ITGA6 in invasive and non-invasive fibroblasts detected by flow cytometry. FIG. 6D depicts Western blot analysis of SEMA7A, F3 and ITGA6 expression in invasive and non-invasive fibroblasts. GAPDH served as the loading control. FIG. 6E shows representative images of migration and invasion of SEMA7A, F3 and ITGA6 negative and high fibroblasts. FIG. 6F shows representative index (SEMA7A Neg/High migration/invasion, n=4 per group; F3 Neg/High migration, n=12 per group, invasion, n=9 per group; ITGA6 Neg/High migration, n=9 per group, invasion, n=12 per group) of migration and invasion of SEMA7A, F3 and ITGA6 negative and high fibroblasts. FIG. 6G depicts a graph showing that IPF lungs showed increased number of SEMA7A+ fibroblasts in IPF lungs by flow cytometry analysis on lung single-cell homogenate in CD31⁻, CD45⁻, EPCAM⁻ cells from IPF (n=8) or healthy (n=4) samples. FIG. 6H shows trichrome staining of mouse lung receiving SEMA7A high and negative fibroblasts and age-matching mice treated with cell culture medium only. FIG. 6I shows hydroxyproline staining (n=10 per group) of mouse lung receiving SEMA7A high and negative fibroblasts. Dash-boxed regions are shown at higher magnification. Scale bar: 1 mm (FIG. 6E), 500 m (FIG. 6H). Data are the mean±SEM, *P<0.05, **P<0.01, ***P<0.001, ****P<0.0001 by student's t test. non-in, non-invasive, in, invasive, Neg, negative.

FIG. 7A-FIG. 7G show cell surface markers used to identify invasive fibroblasts. FIG. 7A shows expression of SEMA7A, F3 and ITGA6 in invasive and non-invasive fibroblasts by qRT-PCR (n=3-6 per group). FIG. 7B shows a heatmap of SEMA7A high, negative and invasive and non-invasive fibroblasts. FIG. 7C shows cell surface expression of CD274, F3 and ITGA6 in SEMA7A negative and high fibroblasts by flow cytometry. FIG. 7D shows cell sorting strategy of F3, SEMA7A and ITGA6 negative and high fibroblasts. FIG. 7E shows RNA level of F3, SEMA7A and ITGA6 expression in sorted F3, SEMA7A and ITGA6 negative and high fibroblasts (n=4 per group). FIG. 7F shows cell adhesion of SEMA7A^{neg}, SEMA7A^{high}, ITGA6^{neg} and ITGA6^{high} fibroblasts was quantified (n=4 per group). FIG. 7G shows cell surface expression of SEMA7A determined by flow cytometry on single-cell homogenate of CD31⁻, CD45⁻, EPCAM⁻ cells from IPF and healthy samples. Data are the mean±SEM, *P<0.05, **P<0.01, ***P<0.001, ****P<0.0001 by student's t test.

FIG. 8A-FIG. 8J show that incRNA, FENDRR was downregulated in invasive lung fibroblasts. FIG. 8A shows visualization of FENDRR and LINC00152 expression in the four clusters using t-SNE plot. non-in, non-invasive; in, invasive. FIG. 8B shows visualization of FENDRR and LINC00152 expression in the four clusters using violin plot. non-in, non-invasive; in, invasive. FIG. 8C shows representative images for RNAscope for FENDRR expression in invasive and non-invasive IPF lung fibroblasts. Data are the mean±SEM. Scale bar: 25 μm. FIG. 8D shows scoring of FIG. 8C (n=3 per group). Scoring categories are condensed to 0-3 and higher score guarantees higher gene expression. *P<0.05, **P<0.01, ***P<0.001 by student's t test. FIG. 8E depicts a graph showing qRT-PCR for FENDRR in invasive and non-invasive lung fibroblasts (n=6 per group). *P<0.05, **P<0.01, ***P<0.001 by student's t test. FIG. 8F depicts a graph showing qRT-PCR for FENDRR in IPF lung fibroblasts treated with si-CTL and si-FENDRR (n=3 per group). *P<0.05, **P<0.01, ***P<0.001 by 1-way ANOVA. non-in, non-invasive; in, invasive. FIG. 8G depicts a representative cell growth curve of lung fibroblasts treated with si-CTL and si-FENDRR. FIG. 8H depicts a graph showing an index of migration of fibroblasts from FIG. 3G treated with si-CTL and si-FENDRR. (n=12 per group). *P<0.05, **P<0.01, ***P<0.001 by 1-way ANOVA. FIG. 8I depicts a graph showing an index of invasion of fibroblasts from FIG. 3G treated with si-CTL and si-FENDRR. (n=12 per group). *P<0.05, **P<0.01, ***P<0.001 by 1-way ANOVA. FIG. 8J shows representative images of lung fibroblasts treated with si-CTL and si-FENDRR from FIG. 8G. Data are the mean±SEM. Scale bar: 1 mm.

FIG. 9A-FIG. 9F show results of knockdown assay of lncRNAs in lung fibroblasts. FIG. 9A shows representative images for knockdown assay of lncRNAs in lung fibroblasts. RNAscope for FENDRR expression in si-CTL and si-FENDRR fibroblasts (n=6-8 per group). Scale bar: 25 μm. FIG. 9B depicts scoring of images of FIG. 9A. RNAscope for FENDRR expression in si-CTL and si-FENDRR fibroblasts (n=6-8 per group). Data are the mean±SEM, *P<0.05, **P<0.01, ***P<0.001, ****P<0.0001 by 1-way ANOVA. FIG. 9C depicts qRT-PCR for LINC00152 expression in invasive and non-invasive fibroblasts (n=6 per group). Data are the mean SEM, *P<0.05, **P<0.01, ***P<0.001, ****P<0.0001 by student's t test. FIG. 9D depicts qRT-PCR for LINC00152 expression in fibroblasts of si-CTL and si-LINC00152 (D) (n=4 per group). Data are the mean±SEM, *P<0.05, **P<0.01, ***P<0.001, ****P<0.0001 by student's t test. FIG. 9E depicts index (E) (n=9 per group) of migration and invasion of SEMA7A high fibroblasts after LINC00152 knockdown and CTL. Data are the mean±SEM, *P<0.05, **P<0.01, ***P<0.001, ****P<0.0001 by student's t test. FIG. 9F shows representative images of migration and invasion of SEMA7A high fibroblasts after LINC00152 knockdown and CTL from FIG. 9E. Scale bar: 1 mm.

FIG. 10A-FIG. 10J depict transcription factors identified to distinguish invasive and non-invasive fibroblasts. FIG. 10A shows visualization of differentially expressing transcription factors, FOXF1, CREBRF, TSC22D1, MXI1, KLF9, and NFE2L2, using t-SNE plot in invasive and non-invasive fibroblast clusters. FIG. 10B shows visualization of differentially expressing transcription factors, HMGA2 and DPF3, using t-SNE plot in invasive and non-invasive fibroblast clusters. FIG. 10C depicts visualization of differentially expressing transcription factors, FOXF1, CREBRF, TSC22D1, MXI, KLF9, and NFE2L2, using violin plot in invasive and non-invasive fibroblast clusters. FIG. 10D depicts visualization of differentially expressing transcription factors, HMGA2 and DPF3, using violin plot in invasive and non-invasive fibroblast clusters. FIG. 10E depicts Western blot analysis of transcription factor expression, FOXF1, CREBRF, TSC22D1, MXI1, KLF9, and NFE2L2, in invasive and non-invasive fibroblasts. GAPDH or 3-tub served as loading control. FIG. 10F depicts Western blot analysis of transcription factor expression, HMGA2 and DPF3, in invasive and non-invasive fibroblasts. GAPDH or 1-tub served as loading control. FIG. 10G depicts Western blot analysis of transcription factor expression after knockdown of each FOXF1, CREBRF, TSC22D1, MXI1, KLF9, and NFE2L2. GAPDH or 1-tub served as loading control. FIG. 10H depicts Western blot analysis of transcription factor expression after knockdown of each HMGA2 and DPF3. GAPDH or 1-tub served as loading control. FIG. 10I shows representative images of migration and invasion of fibroblasts after knockdown assay depicted in FIG. 10G. Scale bar: 1 mm. non-in, non-invasive, in, invasive. FIG. 10J shows representative images of migration and invasion of fibroblasts after knockdown assay depicted in FIG. 10H. Scale bar: 1 mm. non-in, non-invasive, in, invasive.

FIG. 11A-FIG. 11F show that FOXF1 expression showed a negative correlation with SEMA7A expression. FIG. 11A shows relative expression of FOXF1, CREBRF, TSC22D1, MXI1, KLF9, NFE2L2 (A) (n=4-5 per group) in invasive and non-invasive fibroblasts by qRT-PCR. FIG. 11B shows relative expression of HMGA2 and DPF3 (B) (n=6 per group) in invasive and non-invasive fibroblast by qRT-PCR. FIG. 11C shows FOXF1 expression in invasive and noninvasive fibroblasts determined by single cell western blot. β-tubulin served as loading control. FIG. 11D shows FOXF1 and SEMA7A expression correlation in single cell RNA-seq by SeqGeq. FIG. 11E shows relative gene expression of FOXF1, SEMA7A, ACTA2 and COL1A1 in si-CTL and si-FOXF1 lung fibroblasts (n=4 per group). FIG. 11F shows cell surface expression of SEMA7A in si-CTL and si-FOXF1 lung fibroblasts. Data are the mean±SEM, *P<0.05, **P<0.01, ***P<0.001, ****P<0.0001 by 1-way ANOVA.

FIG. 12A-FIG. 12C show migration and invasion index of gene knockdown fibroblasts and control fibroblasts. FIG. 12 A shows relative expression of transcription factors after knockdown by qRT-PCR (n=3-9 per group). FIG. 12B shows migration and invasion ratio of non-invasive-specific transcription factor knockdown fibroblasts (n=3-6 per group) normalized to respective si-CTL lung fibroblasts. FIG. 12C shows migration and invasion ratio of invasive-specific transcription factor knockdown fibroblasts (C) (n=3-6 per group) normalized to respective si-CTL lung fibroblasts. Data are the mean±SEM, *P<0.05, **P<0.01, ***P<0.001, ****P<0.0001 by student's t test.

FIG. 13A-FIG. 13H show that the ERBB2 (HER2) signaling pathway was significantly activated in invasive fibroblasts. FIG. 13A, FIG. 13B, FIG. 13C, and FIG. 13D show that IPA analysis of invasive-1 cluster specific genes identified significantly activated and inhibited upstream regulators. The target gene number and p-value of overlap of the invasive-1 cluster were presented by dot plots. (FIG. 13A, FIG. 13C). The activation Z-score of the invasive-1 cluster was presented by bar graphs. (FIG. 13B, FIG. 13D). ERBB2 (HER2) was highlighted. FIG. 13E shows IPA analysis revealing the upstream regulators of the metastatic cancer cells based on the specific gene expression. FIG. 13F shows analysis of all the shared upstream regulators showing significant positive correlations between invasive fibroblasts and metastatic cancer cells. FIG. 13G shows p-HER2, total HER2 and SEMA7A protein levels in sorted SEMA7A high and negative fibroblasts in 9 IPF fibroblast lines determined by Western blot. GAPDH served as loading control. neg, negative. FIG. 13H shows p-HER2 and total HER2 in sorted fibroblasts from 3 normal and 4 IPF lung determined by Western blot.

FIG. 14A-FIG. 14D show that HER2 inhibitor, Lapatinib, could not change fibroblast survival. FIG. 14A shows ERBB2 (HER2) expression in invasive and non-invasive fibroblasts visualized by t-SNE. FIG. 14B shows ERBB2 (HER2) expression in invasive and non-invasive fibroblasts visualized by violin plots. FIG. 14C shows sorting strategy for normal and IPF lung fibroblasts for p-HER2 and total HER2 western blot. FIG. 14D shows results of calcein AM Assays performed on Lapatinib treated cells for 24 hours to check cell viability. Data are the mean±SEM (n=8 per group).

FIG. 15A-FIG. 15H show that blocking HER2 signaling attenuated fibroblast invasion and lung fibrosis. FIG. 15A shows representative images of migration and invasion of fibroblasts treated with different doses of Lapatinib or DMSO. Scale bar: 1 mm. FIG. 15B and FIG. 15C show an index of migration and invasion of fibroblasts treated with different doses of Lapatinib or DMSO of the images shown in FIG. 15A. Data are the mean±SEM; n=3 per group. **P<0.01, ***P<0.001 and ****P<0.0001 by 1-way ANOVA. FIG. 15D shows representative images of migration and invasion of fibroblasts treated with Pertuzumab or IgG1. Scale bar: 1 mm. FIG. 15E and FIG. 15F show an index of migration of fibroblasts treated with different doses of Pertuzumab or IgG1 shown in FIG. 15D. Data are the mean±SEM; n=3 per group. FIG. 15G shows experimental scheme, and Masson's trichrome staining of collagen in lung sections from NSG mice injected with SEMA7A high IPF fibroblasts treated with Lapatinib and vehicle control. Dash-boxed regions were shown at higher magnification. Scale bar: 500 μm. FIG. 15H shows hydroxyproline content in lung tissues from NSG mice injected with SEMA7A high IPF fibroblasts treated with Lapatinib and vehicle control. Data are the mean±SEM; n=10 per group. **P<0.01, ***P<0.001 and ****P<0.0001 by 2-way ANOVA.

FIG. 16A and FIG. 16B show that targeting HER2 signaling blunted bleomycin-induced lung fibrosis. FIG. 16A shows Masson's trichrome staining of collagen in lung sections from C57BL/6J mice injured with 1.25 U bleomycin and treated with Lapatinib or vehicle control. Dash-boxed regions were shown at higher magnification. Scale bar: 500 m (A). FIG. 16B shows hydroxyproline content in lung tissues from C57BL/6J mice injured with 1.25 U bleomycin and treated with Lapatinib or vehicle control. Dash-boxed regions were shown at higher magnification. Data are the mean±SEM (n=7-9 per group). *P<0.05, and ****P<0.0001 by 2-way ANOVA (B).

FIG. 17 shows that HER2 signaling activation increased fibroblast invasion and fibrosis. (A) The expression of invasive and non-invasive specific genes in HER2 overexpression normal fibroblasts were detected by qRT-PCR. (B) Heatmap of the differentially expressed gene of control and HER2 overexpressed normal human lung fibroblasts by bulk RNA-seq. (C) Volcano plot of the top differentially expressed gene between control and HER2 overexpressed normal human lung fibroblasts by bulk RNA-seq. Red dots indicated the genes at Fold_change>0.5 and black dots indicated the genes at Fold_change<0.5. (D) Relative expression of invasive and non-invasive specific genes in HER2 overexpression normal fibroblasts detected by bulk RNA-seq. (E) Expression of HER2, SEMA7A and FOXF1 in HER2 overexpression normal human lung fibroblasts was confirmed by Western blotting. GAPDH served as loading control. (F) Upregulated cell surface expression of SEMA7A in HER2 overexpression normal lung fibroblasts was confirmed by flow cytometry analysis. (G-H) Representative images (G) and index quantification (H) of normal lung fibroblast invasion after HER2 overexpression. OE, overexpression. Data are the mean±SEM. Scale bar: 1 mm. ns, no significance, *P<0.05, **P<0.01, ***P<0.001, ****P<0.0001 by student's t test. (I-J) Collagen deposition.

FIG. 18 shows that HER2 deficiency rescued the dysregulated gene profiles in IPF lung fibroblasts. (A-C) HER2 knockdown (KD) efficiency was confirmed by qRT-PCR (A) and Western blotting (B, C). (D) Cell surface protein level of HER2 and SEMA7A in HER2 knockdown (KD) IPF lung fibroblasts. (E-F) Representative images (E) and index quantification (F) of fibroblast invasion after HER2 knockdown (KD). (G) Protein levels of phosphorylated HER2 (p-HER2), total HER2, SEMA7A and FOXF1 in IPF lung fibroblasts after treatment of HER2 inhibitor, Lapatinib, at increasing concentration. (H) Downregulation of cell surface expression of SEMA7A, F3 and ITGA6 in Lapatinib treated fibroblasts was determined by flow cytometry analysis. (I) Transcription levels of other representative genes in IPF lung fibroblasts after Lapatinib treatment were determined by qRT-PCR. Scale bar: 1 mm. Data are the mean±SEM. *P<0.05, **P<0.01, ***P<0.001, ****P<0.0001 by student's t test.

FIG. 19 is a graphic summary of HER2 regulation of the gene network in fibroblast invasion and lung fibrosis. HER2 is a master regulator of fibroblast invasion related genes and is activated in invasive fibroblasts and IPF lung fibroblasts. HER2 blockage attenuates lung fibrosis in a humanized IPF model.

### DETAILED DESCRIPTION OF THE INVENTION

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “about” is used herein to mean within the typical ranges of tolerances in the art. For example, “about” can be understood as about 2 standard deviations from the mean. According to certain embodiments, about means +10%. According to certain embodiments, about means +5%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

Ranges provided herein are understood to include all individual integer values and all subranges within the ranges.

As used herein, the term “in combination with,” is not intended to imply that the therapy or the therapeutic agents must be administered at the same time and/or formulated for delivery together, although these methods of delivery are within the scope described herein. The therapeutic agents can be administered concurrently with, prior to, or subsequent to, one or more other additional therapies or therapeutic agents.

The term “active agent” refers to the ingredient, component or constituent of the compositions of the described invention responsible for the intended therapeutic effect.

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “administer” as used herein means to give or to apply. The term “administering,” as used herein, includes in vivo administration, as well as administration directly to tissue ex vivo. “Administering” may be accomplished by any route as disclosed below.

The term “alveolus” or “alveoli,” as used herein, refers to an anatomical structure that has the form of a hollow cavity. Found in the lung, the pulmonary alveoli are spherical outcroppings of the respiratory sites of gas exchange with the blood. The alveoli contain some collagen and elastic fibers. Elastic fibers allow the alveoli to stretch as they fill with air when breathing in. They then spring back during breathing out, in order to expel the carbon dioxide-rich air. The alveolar epithelium comprises two main cell types: the alveolar type I cell and the alveolar type II cell. The alveolar type I (AT1) cell is a complex branched cell with multiple cytoplasmic plates that are greatly attenuated and relatively devoid of organelles; these plates represent the gas exchange surface in the alveolus. Alveolar Type 1 cells are characterized by abundant caveolae and the expression of caveolin 1 protein. The alveolar type II (ATII) cell possesses few caveolae and express little caveolin 1 protein. Alveolar type II cells respond to damage of the vulnerable type I cell by dividing and acting as a progenitor cell for both type I and type II cells. Type II cells are highly metabolic, allowing these cells to perform three important activities that keep the alveolus functioning normally: surfactant production, surfactant homeostasis, and repair of the alveolus following injury

The term “antibody,” as used herein, refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, κ and λ; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies. Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage. The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

As used herein, the terms “antigen” refers to any substance that elicits an immune response.

The term “antigen-binding site,” as used herein, refers to the site at the tip of each arm of an antibody that makes physical contact with an antigen and binds it noncovalently. The antigen specificity of the antigen-binding site is determined by its shape and the amino acids present.

Antigen presenting cells. T cells recognize antigens on the surface of other cells and mediate their functions by interacting with, and altering, the behavior of antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an antigen-presenting cell (APC) that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated. (“Chapter 24: The adaptive immune system,” Molecular Biology of the Cell, Alberts, B. et al., Garland Science, N Y, 2002).

The term “antigenic determinant” or “epitope,” as used herein, refers to that portion of an antigenic molecule that is contacted by the antigen-binding site of a given antibody or antigen receptor.

The term “attenuate” and its various grammatical forms is used herein to refer to weakened or thinned.

The term “β-arrestin,” as used herein, refers to β-arrestins 1 and -2, which are ubiquitously expressed in most mammalian tissues and cell types. β-Arrestins were first thought to only regulate receptor desensitization and internalization. However, it was found that rather than controlling GPCR signal termination, productive β-arrestin dependent GPCR signaling paradigms were highly dependent on multi-protein complex formation and generated long-lasting cellular effects, in contrast to G protein signaling which is transient and functions through soluble second messenger systems. Thus, β-arrestins does not only ‘arrest’ G protein signaling, but it also affects GPCR signaling in a manner independent of G protein-signaling that involves the physical scaffolding of multiple signal transduction proteins where the ‘desensitized’ receptor functioned as a part of a mitogenic signaling complex.

The term “binding” and its other grammatical forms is used to mean a lasting attraction between chemical substances.

The term “binding motif,” as used herein, refers to sequences for specific binding. For example, sequence motifs are short, recurring patterns in DNA that are presumed to have a biological function, which often indicate sequence-specific binding sites for proteins such as nucleases and transcription factors (TF). Transcription factor binding motifs (TFBMs) are genomic sequences that specifically bind to transcription factors.

The term “binding specificity,” as used herein, refers to both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “biocompatible,” as used herein, refers to causing no clinically relevant tissue irritation, injury, toxic reaction, or immunological reaction to living tissue.

The term “biodegradable,” as used herein, refers to material that will break down actively or passively over time by simple chemical processes, by action of body enzymes or by other similar biological activity mechanisms.

The term “biomarker” (or “biosignature”), as used herein, refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes).

The term “bleomycin-induced pulmonary fibrosis model,” as used herein, refers to an animal model of pulmonary fibrosis in rodents. It causes inflammatory and fibrotic reactions within a short period of time, even more so after intratracheal instillation. The initial elevation of pro-inflammatory cytokines (interleukin-1, tumor necrosis factor-α, interleukin-6, interferon-γ) is followed by increased expression of pro-fibrotic markers (transforming growth factor-β1, fibronectin, procollagen-1), with a peak around day 14. The “switch” between inflammation and fibrosis appears to occur around day 9 after bleomycin. While the bleomycin model has the advantage that it is quite easy to perform, widely accessible and reproducible, and therefore fulfills important criteria expected from a good animal model. the bleomycin model has significant limitations in regard to understanding the progressive nature of human IPF. While bleomycin causes an inflammatory response, triggered by overproduction of free radicals, with induction of pro-inflammatory cytokines and activation of macrophages and neutrophils, thus resembling acute lung injury in some way, the subsequent development of fibrosis, however, is at least partially reversible, independent from any intervention, and the aspect of slow and irreversible progression of IPF in patients is not reproduced in the bleomycin model.

The term “bronchoalveolar lavage” (BAL) is used herein to refer to a medical procedure in which a bronchoscope is passed through the mouth or nose into the lungs and fluid is squirted into a small part of the lung and then collected for examination. “Bronchoalveolar lavage fluid” (BALF) is used herein to refer to the fluid collected from a BAL procedure. Bronchoalveolar lavage (BAL), performed during fiberoptic bronchoscopy is a useful adjunct to lung biopsy in the diagnosis of nonneoplastic lung diseases. BAL is able to provide cells and solutes from the lower respiratory tract and may provide important information about diagnosis and yield insights into immunologic, inflammatory, and infectious processes taking place at the alveolar level. BAL has been helpful in elucidating the key immune effector cells driving the inflammatory response in IPF. Increase in polymorphonuclear leukocytes, neutrophil products, eosinophils, eosinophil products, activated alveolar macrophages, alveolar macrophage products, cytokines, chemokines, growth factors for fibroblasts, and immune complexes have been noted in BAL of patients with IPF.

The term “carrier,” as used herein, describes a material that does not cause significant irritation to an organism and does not abrogate the biological activity and properties of the compound of the composition of the described invention. Carriers must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “cell adhesion” is used herein to refer to the process by which cells interact and attach to a surface, substrate or another cell.

The term “cell proliferation,” as used herein, refers to the process that results in an increase of the number of cells, and is defined by the balance between cell divisions and cell loss through cell death or differentiation. Exemplary markers of cell proliferation markers include, but are not limited to, marker of proliferation Ki-67 (MKI67), a nonhistone nuclear protein that is a convenient and reproducible biomarker for this process. Ki-67 protein is present during the active phases of the cell cycle but is absent during the inactive phases of the cell cycle, cyclin-dependent kinase 1 (CDK1), an archetypical kinase and a central regulator that drives cells through G2 phase and mitosis; hyaluronan mediated motility receptor (HIMMR), a hyaluronan receptor distinct from CD44 that compensates for the loss of CD44 in binding hyaluronic acid, and which supports cell migration, and proliferating cell nuclear antigen (PCNA), an evolutionarily well-conserved protein found in all eukaryotic species as well as in Archae that functions in DNA replication, chromatin remodeling, DNA repair, sister-chromatid cohesion and cell cycle control.

The term “chemoattractant,” as used herein, refers to a substance which attracts motile cells of a particular type.

The term “clinical outcome” or “outcome” is used to refer to a specific result or effect that can be measured. Examples of outcomes include progression-free survival and overall survival.

“Cluster of Differentiation” or “cluster of designation” (CD) molecules are utilized in cell sorting using various methods, including flow cytometry. Cell populations usually are defined using a “+” or a “−” symbol to indicate whether a certain cell fraction expresses or lacks a particular CD molecule.

CD31 (platelet/endothelial cell adhesion molecule; PECAM1) normally is found on endothelial cells, platelets, macrophages and Kupffer cells, granulocytes, T cells, natural killer cells, lymphocytes, megakaryocytes, osteoclasts and neutrophils. CD31 has a key role in tissue regeneration and in safely removing neutrophils from the body. Upon contact, the CD31 molecules of macrophages and neutrophils are used to communicate the health status of the neutrophil to the macrophage.

CD44 (hyaluronan receptor) is a cell-surface glycoprotein involved in cell-cell interactions, cell adhesion and migration.

CD45 (protein tyrosine phosphatase, receptor type, C; PTPRC) cell surface molecule is expressed specifically in hematopoietic cells. CD45 is a protein tyrosine phosphatase (PTP) with an extracellular domain, a single transmembrane segment, and two tandem intracytoplasmic catalytic domains, and thus belongs to receptor type PTP. Studies suggest it is an essential regulator of T-cell and B-cell antigen receptor signaling that functions by direct interaction with components of the antigen receptor complexes, or by activating various Src family kinases required for antigen receptor signaling. CD45 also suppresses JAK kinases, and thus functions as a regulator of cytokine receptor signaling. The CD45 family consists of multiple members that are all products of a single complex gene. Various known isoforms of CD45 include: CD45RA, CD45RB, CD45RC, CD45RAB, CD45RAC, CD45RBC, CD45RO, and CD45R (ABC). Different isoforms may be found on different cells. For example, CD45RA is found on naïve T cells and CD45RO is found on memory T cells.

CD49f, also called Integrin Subunit Alpha 6 (ITGA6), is a receptor for laminin on platelets and dimerizes with Integrin Subunit Beta 1 (ITGB1).

CD108, also called Semaphorin 7A (SEMA7A), is a glycosylphosphatidylinositol-anchored Semaphorin that has been previously reported to be regulated by TGF-β1 and plays a critical role in TGF-β1-induced fibrotic responses.

CD142, also known as F3, is a high-affinity receptor for coagulation factor VII and initiates the extrinsic pathway of blood coagulation. CD142 also plays a role in a variety of diseases such as sepsis, atherosclerosis, and cancer.

CD274, also called Programmed death-ligand 1 (PD-L1), is a cell surface protein that binds to PD-1, which is found on T cells, preventing the T cell from killing the cell containing the PD-L1.

The term “compatible,” as used herein, refers to the components of a composition are capable of being combined with each other in a manner such that there is no interaction that would substantially reduce the efficacy of the composition under ordinary use conditions.

The term “component,” as used herein, refers to a constituent part, element or ingredient.

The terms “composition” and “formulation” are used interchangeably herein to refer to a product of the described invention that comprises all active and inert ingredients.

The term “contact” and its various grammatical forms, as used herein, refers to a state or condition of touching or of immediate or local proximity.

The term “co-stimulatory molecule,” as used herein, refers to cell surface proteins on antigen-presenting cells that delivery co-stimulatory signals to naïve T cells. Examples are the B7 molecules on dendritic cells, which are ligands for CD28 on naïve T cells.

The term “co-stimulatory receptors,” as used herein, refers to cell-surface receptors in naïve lymphocytes through which the cells receive signals additional to those received through the antigen receptor and which are necessary for the full activation of the lymphocyte. Examples are CD30 and CD40 on B cells, and CD27 and CD28 on T cells.

The term “cytokine,” as used herein, refers to small soluble protein substances secreted by cells, which have a variety of effects on other cells. Cytokines mediate many important physiological functions, including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

As used herein, the term “derived from” refers to any method for receiving, obtaining, or modifying something from a source of origin.

The term “decrease” and its various grammatical forms is used herein to refer to a diminution, a reduction, an attenuation or abatement of the degree, intensity, extent, size, amount, density or number of occurrences, events or characteristics.

The term “differentiation” is used herein to refer to the process by which an immature cell becomes specialized in order to perform a specific function. According to certain embodiments, “differentiation” refers to fibroblast differentiation in fibrosis.

The term “domain,” as used herein, refers to a portion of a protein that has a tertiary structure of its own. Larger proteins generally are composed of several domains, each connected to the next by short flexible regions of polypeptide chain.

The term “drug,” as used herein, refers to any substance which is intended for use in the diagnosis, cure, mitigation, treatment or prevention of disease or disorder, or to affect the structure or function of the body

The term “effective amount” is used herein to include the amount of an agent that, when administered to a patient for treating a subject having a proliferative tissue fibrosis is sufficient to effect treatment of the disease (e.g., by diminishing, ameliorating or maintaining the existing disease or one or more symptoms of disease or its related comorbidities). The “effective amount” may vary depending on the agent, how it is administered, the disease and its severity and the history, age, weight, family history, genetic makeup, stage of pathological processes, the types of preceding or concomitant treatments, if any, and other individual characteristics of the patient to be treated. An effective amount includes an amount that results in a clinically relevant change or stabilization, as appropriate, of an indicator of a disease or condition. The term includes prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The ERBB2 gene, which is commonly referred to as the HER2 gene, encodes human epidermal growth factor 2. While ErbB2 is used to refer to the gene across both human and rodent species, HER2 is used in reference to the human gene and gene product and neu is used in reference to its rodent counterparts.

The term “expression,” as used herein, generally refers to the action of a gene in the production of a protein or phenotype. More specifically, it refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Expression may also refer to the post-translational modification of a polypeptide or protein.

The term “extracellular matrix” (ECM) is used herein to refer to a network of material such as proteins and polysaccharides that are secreted locally by cells and remain closely associated with them to provide structural, adhesive and biochemical signaling support.

The term “Fab fragment,” as used herein, refers to an antibody fragment composed of a single antigen-binding arm of an antibody without the Fc region, produced by cleavage of IgG by the enzyme papain. It contains the complete light chain plus the amino-terminal variable region and first constant region of the heavy chain, held together by an interchain disulfide bond.

The term “F(ab′)₂ fragment, as used herein, refers to an antibody fragment composed of two linked antigen-binding arms (Fab fragments) without the Fc regions, produced by cleavage of IgG with pepsin.

The term “FENDRR, FOXF1 adjacent noncoding RNA,” as used herein, refers to a spliced long non-coding RNA transcribed bi-directionally with FOXF1 on the opposite strand.

The term “fibroblast,” as used herein, refers to the most common type of cell found in connective tissue. Fibroblasts secrete collagen proteins that are used to maintain a structural framework for many tissues

The term “fibrosis,” as used herein, refers to excessive scarring, which exceeds the normal wound healing response to injury in many tissues. Although the extracellular matrix deposition appears unstructured disrupting the normal tissue architecture and subsequently impairing proper organ function, fibrogenesis is a highly orchestrated process determined by defined sequences of molecular signals and cellular response mechanisms. Persistent injury and parenchymal cell death provokes tissue inflammation, macrophage activation and immune cell infiltration. The release of biologically highly active soluble mediators (alarmins, cytokines, chemokines) lead to the local activation of collagen producing mesenchymal cells such as pericytes, myofibroblasts or Gli1 positive mesenchymal stem cell-like cells, to a transition of various cell types into myofibroblasts as well as to the recruitment of fibroblast precursors and ultimately to loss of function. Fibroproliferative disease may affect almost all tissues and organs, including the skin, kidneys, lungs, cardiac and vascular systems, eyes, liver, pancreas and intestine. Tissue fibrosis is a leading cause of morbidity and mortality.

The term “forced vital capacity,” as used herein, refers to the maximal volume of gas that can be exhaled from full inhalation by exhaling as forcefully and rapidly as possible.

The term “fragment” or “peptide fragment,” as used herein, refers to a small part derived, cut off, or broken from a larger peptide, polypeptide or protein, which retains the desired biological activity of the larger peptide, polypeptide or protein. Examples of antigen-binding fragments of an antibody molecule include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a diabody (dAb) fragment, which consists of a VH domain; (vi) a camelid or camelized variable domain; (vii) a single chain Fv (scFv); (viii) a single domain antibody. Antigen-binding antibody fragments can be produced by recombinant DNA techniques, or by enzymatic or chemical cleavage of intact antibodies and screened for binding in the same manner as are intact antibodies.

The term “gene,” as used herein, refers to a region of DNA that controls a discrete hereditary characteristic, usually corresponding to a single protein or RNA. This definition includes the entire functional unit, encompassing coding DNA sequences, noncoding regulatory DNA sequences, and introns.

The term “growth factor,” as used herein, refers to extracellular polypeptide molecules that bind to a cell-surface receptor triggering an intracellular signaling pathway, leading to proliferation, differentiation, or other cellular response that stimulate the accumulation of proteins and other macromolecules, e.g., by increasing their rate of synthesis, decreasing their rate of degradation, or both. Exemplary growth factors include fibroblast growth factor (FGF), insulin-like growth factor (IGF-1), transforming growth factor beta (TGF-β), and vascular endothelial growth factor (VEGF).

Fibroblast Growth Factor (FGF). The fibroblast growth factor (FGF) family currently has over a dozen structurally related members. FGF1 is also known as acidic FGF; FGF2 is sometimes called basic FGF (bFGF); and FGF7 sometimes goes by the name keratinocyte growth factor. Over a dozen distinct FGF genes are known in vertebrates; they can generate hundreds of protein isoforms by varying their RNA splicing or initiation codons in different tissues. FGFs can activate a set of receptor tyrosine kinases called the fibroblast growth factor receptors (FGFRs). Receptor tyrosine kinases are proteins that extend through the cell membrane. The portion of the protein that binds the paracrine factor is on the extracellular side, while a dormant tyrosine kinase (i.e., a protein that can phosphorylate another protein by splitting ATP) is on the intracellular side. When the FGF receptor binds an FGF (and only when it binds an FGF), the dormant kinase is activated, and phosphorylates certain proteins within the responding cell, activating those proteins.

FGFs are associated with several developmental functions, including angiogenesis (blood vessel formation), mesoderm formation, and axon extension. While FGFs often can substitute for one another, their expression patterns give them separate functions. For example, FGF2 is especially important in angiogenesis, whereas FGF8 is involved in the development of the midbrain and limbs.

The term “hepatocyte growth factor” (or HGF), as used herein, refers to a pleiotrophic growth factor, which induces cellular motility, survival, proliferation, and morphogenesis, depending upon the cell type. In the adult, HGF has been demonstrated to play a critical role in tissue repair, including in the lung. Administration of HGF protein or ectopic expression of HGF has been demonstrated in animal models of pulmonary fibrosis to induce normal tissue repair and to prevent fibrotic remodeling. HGF-induced inhibition of fibrotic remodeling may occur via multiple direct and indirect mechanisms including the induction of cell survival and proliferation of pulmonary epithelial and endothelial cells, and the reduction of myofibroblast accumulation.

Insulin-Like Growth Factor (IGF-1). IGF-1, a hormone similar in molecular structure to insulin, has growth-promoting effects on almost every cell in the body, especially skeletal muscle, cartilage, bone, liver, kidney, nerves, skin, hematopoietic cell, and lungs. It plays an important role in childhood growth and continues to have anabolic effects in adults. IGF-1 is produced primarily by the liver as an endocrine hormone as well as in target tissues in a paracrine/autocrine fashion. Production is stimulated by growth hormone (GH) and can be retarded by undernutrition, growth hormone insensitivity, lack of growth hormone receptors, or failures of the downstream signaling molecules, including tyrosine-protein phosphatase nonreceptor type 11 (also known as SHP2, which is encoded by the PTPN11 gene in humans) and signal transducer and activator of transcription 5B (STAT5B), a member of the STAT family of transcription factors. Its primary action is mediated by binding to its specific receptor, the Insulin-like growth factor 1 receptor (IGF1R), present on many cell types in many tissues. Binding to the IGF1R, a receptor tyrosine kinase, initiates intracellular signaling; IGF-1 is one of the most potent natural activators of the AKT signaling pathway, a stimulator of cell growth and proliferation, and a potent inhibitor of programmed cell death. IGF-1 is a primary mediator of the effects of growth hormone (GH). Growth hormone is made in the pituitary gland, released into the blood stream, and then stimulates the liver to produce IGF-1. IGF-1 then stimulates systemic body growth. In addition to its insulin-like effects, IGF-1 also can regulate cell growth and development, especially in nerve cells, as well as cellular DNA synthesis.

IGF-1 was shown to increase the expression levels of the chemokine receptor CXCR4 (receptor for stromal cell-derived factor-1, SDF-1) and to markedly increase the migratory response of MSCs to SDF-1. The IGF-1-induced increase in MSC migration in response to SDF-1 was attenuated by PI3 kinase inhibitor (LY294002 and wortmannin) but not by mitogen-activated protein/ERK kinase inhibitor PD98059. Without being limited by any particular theory, the data indicate that IGF-1 increases MSC migratory responses via CXCR4 chemokine receptor signaling which is PI3/Akt dependent.

The term “platelet derived growth factor” or “PDGF,” as used herein, refers to a major mitogen for connective tissue cells and certain other cell types. It is a dimeric molecule consisting of disulfide-bonded, structurally similar A- and B-polypeptide chains, which combine to homo- and heterodimers. The PDGF isoforms exert their cellular effects by binding to and activating two structurally related protein tyrosine kinase receptors, denoted the alpha-receptor and the beta-receptor. Activation of PDGF receptors leads to stimulation of cell growth, but also to changes in cell shape and motility; PDGF induces reorganization of the actin filament system and stimulates chemotaxis, i.e., a directed cell movement toward a gradient of PDGF. In vivo, PDGF has important roles during the embryonic development as well as during wound healing. Moreover, overactivity of PDGF has been implicated in several pathological conditions.

Transforming Growth Factor Beta (TGF-β). There are over 30 structurally related members of the TGF-β superfamily, and they regulate some of the most important interactions in development. The proteins encoded by TGF-β superfamily genes are processed such that the carboxy-terminal region contains the mature peptide. These peptides are dimerized into homodimers (with themselves) or heterodimers (with other TGF-β peptides) and are secreted from the cell. The TGF-β superfamily includes the TGF-β family, the activin family, the bone morphogenetic proteins (BMPs), the Vg-1 family, and other proteins, including glial-derived neurotrophic factor (GDNF, necessary for kidney and enteric neuron differentiation) and Müllerian inhibitory factor, which is involved in mammalian sex determination. TGF-β family members TGF-β1, 2, 3, and 5 are important in regulating the formation of the extracellular matrix between cells and for regulating cell division (both positively and negatively). TGF-β1 increases the amount of extracellular matrix epithelial cells make both by stimulating collagen and fibronectin synthesis and by inhibiting matrix degradation. TGF-βs may be critical in controlling where and when epithelia can branch to form the ducts of kidneys, lungs, and salivary glands.

The term tumor necrosis factor-alpha (“TNF-α”), as used herein, refers to a potent pro-inflammatory cytokine exerting pleiotropic effects on various cell types and plays a critical role in the pathogenesis of chronic inflammatory diseases. Transmembrane TNF-α, a precursor of the soluble form of TNF-α, is expressed on activated macrophages and lymphocytes as well as other cell types. After processing by TNF-α-converting enzyme (TACE), the soluble form of TNF-α is cleaved from transmembrane TNF-α and mediates its biological activities through binding to Types 1 and 2 TNF receptors (TNF-R1 and -R2) of remote tissues. Accumulating evidence suggests that not only soluble TNF-α, but also transmembrane TNF-α is involved in the inflammatory response.

Vascular Endothelial Growth Factor (VEGF). VEGFs are growth factors that mediate numerous functions of endothelial cells including proliferation, migration, invasion, survival, and permeability. The VEGFs and their corresponding receptors are key regulators in a cascade of molecular and cellular events that ultimately lead to the development of the vascular system, either by vasculogenesis, angiogenesis, or in the formation of the lymphatic vascular system. VEGF is a critical regulator in physiological angiogenesis and also plays a significant role in skeletal growth and repair.

VEGF's normal function creates new blood vessels during embryonic development, after injury, and to bypass blocked vessels. In the mature established vasculature, the endothelium plays an important role in the maintenance of homeostasis of the surrounding tissue by providing the communicative network to neighboring tissues to respond to requirements as needed. Furthermore, the vasculature provides growth factors, hormones, cytokines, chemokines and metabolites, and the like, needed by the surrounding tissue and acts as a barrier to limit the movement of molecules and cells.

The term “hyaluronan synthase 2” (Has2), as used herein, refers to an enzyme involved in hyaluronan/hyaluronic acid (HA) synthesis.

HER2 signaling pathway. The human epidermal growth factor receptor 2 (HER2, also known as ERBB2) proto-oncogene encodes a 185 kDa glycoprotein receptor tyrosine kinase, which is a member of the growth factor receptor family that includes four distinct receptors: EGFR/ErbB-1, HER2/ErbB-2, HER3/ErbB-3, and HER4/ErbB-4. Both homo- and heterodimers are formed by the four members of the EGFR family, with HER2 being the preferred and most potent dimerization partner for other ErbB receptors. HER2 can be activated by overexpression or by heterodimerization with other ErbBs that can be activated by ligand binding. HER2 activation leads to receptor phosphorylation, which triggers a cascade of downstream signals through multiple signaling pathways, such as MAPK, phosphoinositol 3-kinase/AKT, JAK/STAT and PKC, which ultimately results in the regulation of multiple cellular functions, such as growth, survival and differentiation.

“HER2 blocking agents” refer to a class of molecules that inhibit (block) a biologic activity or process mediated by HER2.

Idiopathic Pulmonary fibrosis (IPF, also known as cryptogenic fibrosing alveolitis, CFA, or Idiopathic Fibrosing Interstitial Pneumonia) is defined as a specific form of chronic, progressive fibrosing interstitial pneumonia of uncertain etiology that occurs primarily in older adults, is limited to the lungs, and is associated with the radiologic and histological pattern of usual interstitial pneumonia (UIP). It may be characterized by abnormal and excessive deposition of fibrotic tissue in the pulmonary interstitium. On high-resolution computed tomography (HRCT) images, UIP is characterized by the presence of reticular opacities often associated with traction bronchiectasis. As IPF progresses, honeycombing becomes more prominent. Pulmonary function tests often reveal restrictive impairment and reduced diffusing capacity for carbon monoxide. Studies have reported significant increases in TNF-α and IL-6 release in patients with idiopathic pulmonary fibrosis (IPF), which has been attributed to the level of expression of IL-1β. The onset of IPF symptoms, shortness of breath and cough, are usually insidious but gradually progress, with death occurring in 70% of patients within five years after diagnosis.

The term “improves” is used to convey that the present invention changes either the appearance, form, characteristics and/or the physical attributes of the subject, organ, tissue or cell to which it is being provided, applied or administered. For example, and without limitation, the change in form may be demonstrated by any of the following alone or in combination: a decrease in one or more symptoms of a disease or disorder; increase in renewal of alveolar epithelial cell 2 (AEC2) cells; repair of a lung injury; reduce lung fibrosis; reduction or elimination of the need for other active agents or therapeutics; and slower progression of fibrotic lung disease.

The term “inflammation,” as used herein, refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation,” as used herein, refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation,” as used herein, refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inhibit” and its various grammatical forms are used herein to refer to reducing the amount or rate of a process, to stopping the process entirely, or to decreasing, limiting, or blocking the action or function thereof. Inhibition may include a reduction or decrease of the amount, rate, action function, or process of a substance by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99%.

The term “inhibitor,” as used herein, refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

The term “injury,” as used herein, refers to damage or harm to a structure or function of the body caused by an outside agent or force, which may be physical or chemical.

The term “interleukin” is used herein to refer to a cytokine secreted by leukocytes as a means of communication with other leukocytes Interleukins regulate cell growth, differentiation, and motility, and stimulates immune responses, such as inflammation. Examples of interleukins include, interleukin-1 (IL-1), interleukin-1β (IL-1β), interleukin-6 (IL-6), interleukin-8 (IL-8), and interleukin-12 (IL-12).

The term “interstitial lung disease (ILD),” as used herein, refers to a large number of chronic lung disease characterized by varying degrees of inflammation and fibrosis.

The term “invasion” or “invasiveness” is used herein to refer to a process that includes penetration of and movement of cells through surrounding tissues.

The term “isoform,” as used herein, refers to any of two or more functionally similar proteins that have a similar but not identical amino acid sequence and are either encoded by different genes or by RNA transcripts from the same gene which have had different exons removed (spice variants).

The JAK/STAT pathway is the signaling cascade induced by cytokines that signal via this pathway. The signaling chains of the hematopoietin family of cytokine receptors are noncovalently associated with protein tyrosine kinases of the Janus kinase (JAK) family, which have two tandem kinase-like domains. There are four members of the JAK family: Jak1, Jak2, Jak3, and Tyk2. The dimerization or clustering of receptor signaling chains brings the JAKs into close proximity, causing phosphorylation of each JAK on a tyrosine residue that stimulates its kinase activity. The activated JAKs then phosphorylate their associated receptors on specific tyrosine residues. This phosphotyrosine, and the specific amino acid sequence surrounding it, creates a binding site that is recognized by SH2 domains found in other proteins, in particular members of a family of transcription factors known as signal transducers and activators of transcription (STATs). There are seven STATs (1-4, 5a, 5b, and 6), which reside in the cytoplasm in an inactive form until activated by cytokine receptors. Before activation, most STATS form homodimers, due to a specific homotypic interaction between domains present at the amino termini of the individual STAT proteins. The receptor specificity of each STAT is determined by the recognition of the distinctive phosphotyrosine sequence on each activated receptor by the different SH2 domains within the various STAT proteins. Recruitment of a STAT to the activated receptor brings the STAT close to an activated Janus kinase (JAK), which can then phosphorylate a conserved tyrosine residue in the carboxy terminus of the particular STAT. This leads to a rearrangement, in which the phosphotyrosine of each STAT protein binds to the SH2 domain of the other STAT, forming a configuration that can bind DNA with high affinity. Activated STATS predominantly form homodimers, with a cytokine typically activating one type of STAT. For example, IFN-gamma activates STAT1 and generates STAT 1 homodimers, while IL-4 activates STAT6, generating STAT1 homodimers. Other cytokine receptors can activate several STATS, and some STAT heterodimers can be formed. The phosphorylated STAT dimer enters the nucleus, where it acts as a transcription factor to initiate the expression of selected genes that can regulate growth and differentiation of particular subsets of lymphocytes. Since signaling by these receptors depends on tyrosine phosphorylation, dephosphorylation of the receptor complex by tyrosine phosphatases is one way that cells can terminate signaling, e.g., the nonreceptor tyrosine phosphatases SHP-1 and SHP2 (encoded by PTPN6 and PTPN11) and the transmembrane receptor tyrosine phosphatase CD45). Cytokine signaling can also be terminated by negative feedback involving specific inhibitors that are induced by cytokine activation. Suppressor of cytokine signaling (SOCS) proteins contain an SH2 domain that can recruit them to the phosphorylated JAK kinase or receptor; they can inhibit JAK kinases directly, compete for the receptor, and direct the ubiquitination and subsequent degradation of JAKs and STATs. SOCS proteins are induced by STAT activation, and thus inhibit receptor signaling after the cytokine has had its effect. Another class of inhibitory proteins consists of the protein inhibitors of activated STAT (PIAS) proteins, which also seem to be involved in promoting the degradation of receptors and pathway components.'

The terms LINC00152 and long intergenic non-coding RNA 152, as used herein, refer to a long intergenic non-coding RNA that, can promote cell proliferation.

The term “long intervening non-coding RNAs” (or “lincRNA”), as used herein, to refer to a population of non-coding RNAs which do not overlap with the coding region, while lncRNAs can overlap with coding regions.

The term “long non-coding RNA” (“lncRNAs”), as used herein, refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression

The term “lung function” is used herein to refer to a measure of how well the lung is working. Several types of lung function tests may be conducted, including spirometry, pulse oximetry, exercise stress test or arterial blood gas test. Additionally, hydroxyproline levels, lung density and total cell count in bronchoalveolar lavage fluid may be used to assess lung function. It is to be understood that any one of these tests may be used in combination with another.

For example, lung function may be assessed by determining the amounts of polymorphonuclear leukocytes, neutrophil products, eosinophils, eosinophil products, activated alveolar macrophages, alveolar macrophage products, cytokines, chemokines, growth factors for fibroblasts, and immune complexes in BAL fluid in an untreated control, or relative to a patient at a time point prior to treatment, where a decrease in the amounts of polymorphonuclear leukocytes, neutrophil products, eosinophils, eosinophil products, activated alveolar macrophages, alveolar macrophage products, cytokines, chemokines, growth factors for fibroblasts, and/or immune complexes is indicative of an increase in lung function.

Hydroxyproline is a major component of collagen, where it serves to stabilize the helical structure. Because hydroxyproline is largely restricted to collagen, the measurement of hydroxyproline levels can be used as an indicator of collagen content. A decrease in hydroxyproline levels relative to an untreated control subject with pulmonary fibrosis, or relative to a subject with pulmonary fibrosis at a time point prior to treatment, are indicative of an increase in lung function.

Pulmonary fibrosis involves gradual exchange of normal lung parenchyma with fibrotic tissue, which corresponds with an increase in lung density. Increase in lung density can be measured by computed tomography (CT). A decrease in lung density relative to an untreated control, or relative to a patient at a time point prior to treatment, are indicative of an increase in lung function.

The terms “lung interstitium” or “pulmonary interstitium” are used interchangeably herein to refer to an area located between the airspace epithelium and pleural mesothelium in the lung. Fibers of the matrix proteins, collagen and elastin, are the major components of the pulmonary interstitium. The primary function of these fibers is to form a mechanical scaffold that maintains structural integrity during ventilation.

The abbreviation “MAPK,” as used herein, refers to Mitogen-Activated Protein Kinase (MAPK) signaling which activates a three-tiered cascade with MAPK kinase kinases (MAP3K) activating MAPAK kinases (MAP2K) and finally MAPK. MAPKs are protein Ser/Thr kinases that convert extracellular stimuli into a wide range of cellular responses. The major MAPK pathways involved in inflammatory diseases are extracellular regulating kinase (ERK), p38 MAPK, and c-Jun NH2-terminal kinase (INK). All three MAPK pathways may be activated by TGF-β, and signaling through these cascades can further regulate the expression of Smad proteins and mediate Smad-independent TGF-β responses. These three MAPK pathways are all involved in TGF-β-induced fibrosis. Upstream kinases include TGFβ-activated kinase-1 (TAK1) and apoptosis signal-regulating kinase-1 (ASK1). Downstream of p38 MAPK is MAPK activated protein kinase 2 (MAPKAPK2 or MK2). TGF-β can signal in a noncanonical manner via the MAPK family.

The term “matrix,” as used herein, refers to a three dimensional network of fibers that contains voids (or “pores”) where the woven fibers intersect. The structural parameters of the pores, including the pore size, porosity, pore interconnectivity/tortuosity and surface area, can affect how substances (e.g., fluid, solutes) move in and out of the matrix.

The term “maximum tolerated dose,” as used herein, refers to the highest dose of a drug that does not produce unacceptable toxicity.

The term “messenger RNA” (“mRNA”), as used herein, refers to a coding RNA, which functions in protein translation.

The term “microRNA” (or “miRNA” or “miR”), as used herein, refers to a class of small, 18- to 28-nucleotide-long, noncoding RNA molecules.

The term “migration” is used herein to refer to movement of a cell from one place or location to another.

The term “modulate” and its various grammatical forms is used to refer to adjusting, or changing.

By “naturally occurring” or “wild type,” or grammatical equivalents herein is meant an amino acid sequence or a nucleotide sequence that is found in nature and includes allelic variations; that is, an amino acid sequence or a nucleotide sequence that usually has not been intentionally modified. Accordingly, by “non-naturally occurring” or “synthetic” or “recombinant” or grammatical equivalents thereof herein is meant an amino acid sequence or a nucleotide sequence that is not found in nature; that is, an amino acid sequence or a nucleotide sequence that usually has been intentionally modified. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purpose of the invention.

The term “nonlinear dimensionality reduction” (e.g., t-distributed stochastic neighbor embedding (t-SNE)), as used herein, refers to an algorithm for dimensionality reduction that is particularly well suited for the visualization of high-dimensional datasets. The algorithm applies a non-linear dimensionality reduction technique by keeping very similar data points close together in lower-dimensional space.

The abbreviation “NFκB,” as used herein, refers to which is a proinflammatory transcription factor. It switches on multiple inflammatory genes, including cytokines, chemokines, proteases, and inhibitors of apoptosis, resulting in amplification of the inflammatory response. The molecular pathways involved in NF-κB activation include several kinases. The classic (canonical) pathway for inflammatory stimuli and infections to activate NF-κB signaling involve the TKK (inhibitor of κB kinase) complex, which is composed of two catalytic subunits, IKK-α and IKK-β, and a regulatory subunit IKK-γ (or NFκB essential modulator. The IKK complex phosphorylates Nf-κB-bound IκBs, targeting them for degradation by the proteasome and thereby releasing NF-κB dimers that are composed of p65 and p50 subunits, which translocate to the nucleus where they bind to κB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes.

The term “overall survival” (or “OS”), as used herein, refers to the length of time from either the date of diagnosis or the start of treatment for a disease that patients diagnosed with the disease are still alive. “Overall survival rate” refers to the percentage of people in a study or treatment group who are still alive for a certain period of time after they were diagnosed with or started treatment for a disease.

The term “parenteral,” as used herein, refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques.

The term “pharmaceutical composition” is used herein to refer to a composition that is employed to prevent, reduce in intensity, cure or otherwise treat a target condition or disease. The terms “formulation” and “composition” are used interchangeably herein to refer to a product of the described invention that comprises all active and inert ingredients.

The term “pharmaceutically acceptable” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

As used herein, each of the terms “peptide,” “polypeptide” and “protein” refer to two or more amino acids covalently linked by an amide bond or non-amide equivalent. A peptide generally is considered an amino acid polymer of 40 or fewer amino acids. A polypeptide generally is considered an amino acid polymer containing more than 40 amino acids but less than 100 amino acids. A protein generally is considered an amino acid polymer with a defined sequence that is greater than 100 amino acids in size. The amino acid polymers of the present disclosure can be of any length. For example, they can have from about two to about 100 or more residues, such as, 5 to 12, 12 to 15, 15 to 18, 18 to 25, 25 to 50, 50 to 75, 75 to 100, 100 to 200, or more in length. The amino acid sequence can include L- and D-isomers, and combinations of L- and D-isomers. The amino acid sequence also can include modifications typically associated with post-translational processing of proteins, for example, cyclization (e.g., disulfide or amide bond), phosphorylation, glycosylation, carboxylation, ubiquitination, myristylation, or lipidation.

The term “pharmaceutically acceptable” is used to refer to the carrier, diluent or excipient being compatible with the other ingredients of the formulation or composition and not deleterious to the recipient thereof. For example, the term “pharmaceutically acceptable” can mean approved by a regulatory agency of the Federal or a state government or listed in the U.S. Pharmacopeia or other generally recognized pharmacopeia for use in animals, and more particularly in humans.

“Phosphopinositide 3-Kinase Pathway, AKT/mTOR and PAK2/c-Abl”. The phosphoatidylinositol 3-kinase (PI3K) pathway is a non-Smad pathway contributing to TGF-β induced fibrosis. It induces two profibrotic pathways: Akt-mammalian target of rapamycin (mTOR) and p21-activated kinase 2 (PAK2)/Abelson kinase (c-Abl). The phosphatidylinositol-3-kinase (PI3K)/Akt and the mammalian target of rapamycin (mTor) signaling pathways are crucial to many aspects of cell growth and survival. “They are so interconnected that they could be regarded as a single pathway that, in turn, heavily interacts with many other pathways, including that of hypoxia inducible factors (HIFs).

PI3Ks constitute a lipid kinase family characterized by the capability to phosphorylate inositol ring 3′—OH group in inositol phospholipids. Class I PI3Ks are heterodimers composed of a catalytic (CAT) subunit (i.e., p110) and an adaptor/regulatory subunit (i.e., p85). This classis further divided into two subclasses: subclass IA (PI3Kα, β, and δ), which is activated by receptors with protein tyrosine kinase activity, and subclass IB (PI3Kγ), which is activated by receptors coupled with G proteins.

Activation of growth factor receptor protein tyrosine kinases results in autophosphorylation on tyrosine residues. P13K is then recruited to the membrane by directly binding to phosphotyrosine consensus residues of growth factor receptors or adaptors through one of the two SH2 domains in the adaptor subunit. This leads to allosteric activation of the CAT subunit. PI3K activation leads to the production of the second messenger phosphatidylinositol-4,4-bisphosphate (PI3,4,5-P3) from the substrate phosphatidylinositol-4,4-bisphosphate (PI-4,5-P2). PI3,4,5-P3 then recruits a subset of signaling proteins with pleckstrin homology (PH) domains to the membrane, including protein serine/threonine kinase-3′-phosphoinositide-dependent kinase I (PDK1) and Akt/protein kinase B (PKB). Akt/PKB, on its own, regulates several cell processes involved in cell survival and cell cycle progression.

Akt. Akt (also known as protein kinase B) is a 60 kDa serine/threonine kinase. It is activated in response to stimulation of tyrosine kinase receptors such as platelet-derived growth factor (PDGF), insulin-like growth factor, and nerve growth factor. Stimulation of Akt has been shown to be dependent on phosphatidylinositol 3-kinase (PI3-kinase) activity.

Akt has been shown to act as a mediator of survival signals that protect cells from apoptosis in multiple cell lines. For example, phosphorylation of the pro-apoptotic Bad protein by Akt was found to decrease apoptosis by preventing Bad from binding to the anti-apoptotic protein Bcl-XL. Akt was also shown to promote cell survival by activating nuclear factor-kB (NF-kB) and inhibiting the activity of the cell death protease caspase-9.

mTOR signaling pathway: Mechanistic target of rapamycin (mTOR) is an atypical serine/threonine kinase that is present in two distinct complexes. The first, mTOR complex 1 (mTORC1), is composed of mTOR, Raptor, GβL, and DEPTOR and is inhibited by rapamycin. It is a master growth regulator that senses and integrates diverse nutritional and environmental cues, including growth factors, energy levels, cellular stress, and amino acids. It couples these signals to the promotion of cellular growth by phosphorylating substrates that potentiate anabolic processes such as mRNA translation and lipid synthesis, or limit catabolic processes such as autophagy. The small GTPase Rheb, in its GTP-bound state, is a necessary and potent stimulator of mTORC1 kinase activity, which is negatively regulated by its GTPase-activating protein (GAP), the tuberous sclerosis heterodimer TSC1/2. TSC1 and TSC2 are the tumour-suppressor genes mutated in the tumour syndrome TSC (tuberous sclerosis complex). Their gene products form a complex (the TSC1-TSC2 (hamartin-tuberin) complex), which, through its GAP activity towards the small G-protein Rheb (Ras homologue enriched in brain), is a critical negative regulator of mTORC1 (mammalian target of rapamycin complex 1). Most upstream inputs are funneled through Akt and TSC1/2 to regulate the nucleotide-loading state of Rheb. In contrast, amino acids signal to mTORC1 independently of the PI3K/Akt axis to promote the translocation of mTORC1 to the lysosomal surface where it can become activated upon contact with Rheb. This process is mediated by the coordinated actions of multiple complexes, including the v-ATPase, Ragulator, the Rag GTPases, and GATOR1/2. The second complex, mTOR complex 2 (mTORC2), is composed of mTOR, Rictor, GOL, Sin1, PRR5/Protor-1, and DEPTOR. mTORC2 promotes cellular survival by activating Akt, regulates cytoskeletal dynamics by activating PKCα, and controls ion transport and growth via SGK1 phosphorylation. Aberrant mTOR signaling is involved in many disease states

PI3K also acts as a branch point in response to TGF-β, leading to activation of PAK2/c-Abl, which stimulates collagen gene expression in normal fibroblasts, and induces fibroblast proliferation, thereby increasing the number of myofibroblast precursors. PAK2/c-Abl promotes fibrosis through its downstream mediators, including PKCδ/Fli-1 and early growth response (Egr)-1, -2, and -3.

The term “Programmed cell Death protein 1” (known also as “PD-1” and “CD274”) is a type I transmembrane protein preferentially expressed in immune cells such as T, B and NK cells. PD-Ligand 1 (PD-L1, also known as CD274, B7-H1, PDCDIL1, PDCDILG1, and PDL1) is a member of the B7 family of co-stimulatory/co-inhibitory molecules of antigen presentation expressed by a wide range of cell types, including cancer cells. PD1 is made of an extracellular immunoglobulin-like binding domain, a transmembrane region and a cytoplasmic domain containing an immunoreceptor tyrosine-based inhibitory motif (ITIM) and an immunoreceptor tyrosine-based switch motif (ITSM). These motifs are implicated in its immunosuppressive effects. When engaged to its receptor PD-L1, PD-1 strongly interferes with T cell receptor (TCR) signal transduction through several poorly understood molecular mechanisms. Interfering with PD-1 signal transduction either by antibody blockade or any other means enhances T cell functions by potentiating signal transduction from the TCR signalosome. PD-L-PD-1 interactions lead to cell cycle arrest in G0/G1 but do not increase cell death. In addition, ligation of PD-1+TCR leads to rapid phosphorylation of SHP-2, as compared to TCR ligation alone, and inhibits T cell activation.

PD-L2 is a second ligand for PD-1. Engagement of PD-1 by PD-L2 dramatically inhibits T cell receptor (TCR)-mediated proliferation and cytokine production by CD4+ T cells. At low antigen concentrations, PD-L2-PD-1 interactions inhibit strong B7-CD28 signals. In contrast, at high antigen concentrations, PD-L2-PD-1 interactions reduce cytokine production but do not inhibit T cell proliferation. At low antigen concentrations, PD-L2-PD-1 interactions inhibit strong B7-CD28 signals. In contrast, at high antigen concentrations, PD-L2-PD-1 interactions reduce cytokine production but do not inhibit T cell proliferation.

The term “progression” is used herein to refer to the course of a disease as it becomes worse.

The term “progression-free survival” (or PFS) is used herein to refer to the length of time during and after the treatment of a disease that a patient lives with the disease but it does not get worse.

Protein kinase C (PKC) is a multi-gene family of serine/threonine protein kinases that plays critical roles in signal transduction and cell regulation. It can be categorized into three groups based on their structural variations and biochemical properties. The classical or conventional (c) PKCs (α, βI, βII, γ) require Ca2+ and DAG/phorbol esters for their activities. The novel (n) (δ, ε, η, θ) PKCs are insensitive to Ca2+ but respond to DAG/phorbol esters. The atypical (a) PKCs (ξ, ξ) are insensitive to both Ca2+ and DAG/phorbol esters. While the physiological stimulator DAG causes transient activation of conventional and novel PKCs, the tumor-promoting phorbol esters cause persistent activation. Activation of PKCs induces their translocation to the membrane followed by their degradation or downregulation. Protein kinase C3 controls NFκB activation in B cells through selective phosphorylation of the IκB kinase (IKK) alpha subunit.

The term “pulmonary fibrosis” or “PF,” as used herein, refers to a progressive chronic inflammatory disease with a poor clinical outcome. Pulmonary fibrosis (PF) occurs in association with a wide range of diseases, including scleroderma (systemic sclerosis), sarcoidosis, and infection, and as a result of environmental exposures (e.g., silica dust or asbestos), but in most patients it is idiopathic and progressive. Pulmonary fibrosis is characterized by parenchymal honeycombing (meaning the characteristic appearance of variably sized cysts in a background of densely scarred lung tissue. Microscopically, enlarged airspaces surrounded by fibrosis with hyperplastic or bronchiolar type epithelium are present. (From https://emedicine.medscape.com/article/2078590-overview), reduced lung compliance, and restrictive lung function (meaning a decreased lung capacity or volume, so a person's breathing rate often increases to meet the oxygen needs on inhalation). Fibrosis of the interstitial spaces (meaning the walls of the air sacs of the lungs (alveoli) and the spaces around blood vessels and small airways) hinders gas exchange, culminating in abnormal oxygenation and clinical dyspnea (meaning shortness of breath, inability to take a deep breath, or chest tightness). Progressive pulmonary fibrosis also leads to pulmonary hypertension, right-sided heart failure, and ultimately respiratory failure.

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

The term “reduce” and its various grammatical forms is used herein to refer to a diminution, a decrease, an attenuation or abatement of the degree, intensity, extent, size, amount, density or number of occurrences, events or characteristics.

The term “repair,” as used herein, as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function.

The term “RNA molecule” or “ribonucleic acid molecule,” as used herein, refers to a linear, single-stranded polymer composed of ribose nucleotides, that is synthesized by transcription of DNA or by copying of RNA. It encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties.

The term SERPINE1, as used herein, refers to a gene that encodes plasminogen activator inhibitor 1 (PAI-1). PAI-1 is involved in normal blood clotting (hemostasis).

The term “signature,” as used herein, refers to a specific and complex combination of biomarkers that reflect a biological state.

The term “single-cell regulome data analysis toolbox” (or “SCRAT”), as used herein, refers to software with a graphical user interface for studying cell heterogeneity according to single-cell mapping technologies and based on self-organizing maps (SOM) machine learning. SCRAT takes sequences and aggregates the sequences from each single cell according to a chosen feature (e.g., binding motifs, genes, domains, etc.) and normalizes the reads to adjust for library size. Cells are clustered into subpopulations based on chosen features to determine cell population heterogeneity. Clustered cell subpopulations can be compared to cellular identity accumulated in a database. (Ji, Z et al. Bioinfomatics (2017) 33(18): 2930-2932).

The term “single-cell RNA sequencing” (or “scRNA-seq”), as used herein, describes techniques used to sequence RNA from multiple cells each identified by a unique barcoded tag. Identified RNA sequences are categorized by the cell of origin. The transcriptomes of a cell population can be sequenced in parallel. Sequence RNA is used as input for SCAT. GemCode™ (scRNA-seq; 10× Genomics) is a “gel bead in emulsion” method of scRNA-seq. RNAs from an individual cell are tagged with the same unique barcodes and distinguishable from other cells.

The term “signal molecule,” as used herein, refers to an extracellular or intracellular molecule that cues the response of a cell to the behavior of other cells or objects in the environment, e.g., by binding to a receptor. The term “signal transduction” as used herein refers to the relaying of a signal by conversion from one physical chemical form to another. In cell biology, it refers to the process by which a cell converts an extracellular signal into a response.

The term “small molecule inhibitor” is used herein to refer to a low molecular weight (<900 Daltons) organic compound that is used to inhibit a disease or process in a subject,

Smad-dependent pathway for TGF-β signaling. The classical Smad-dependent pathway for transforming growth factor-β signaling occurs when TGF-β receptor type 2, which is constitutively active, transphosphorylates and forms a complex with the TGF-β-bound TGF-β receptor type 1. This complex then phosphorylates serine residues of cytoplasmic receptor-activated Smad (R-Smad), a complex of Smad2 and Smad3. These two heterodimerize and bind to the common mediator Smad (Co-Smad) Smad4, and the whole complex translocates across the nuclear membrane to interact with specific cis-acting elements in the regulatory regions of its target genes, recruiting coactivators such as p300 and CBP; corepressors such as c-Ski, SnoN, transforming growth-inhibiting factor, and Smad nuclear-interacting protein 1; or transcription factors such as AP-1 and Sp1 to modulate gene expression. Inhibitory Smad (I-Smad) Smad6 or Smad7, acting as negative regulators, not only antagonizes the TGF-β/Smad pathway by binding to TGF-β1 or competing with activated R-Smad for binding to Co-Smad, but also recruits the E3 ubiquitin-protein ligases Smurf1 and Smurf2, which target Smad proteins for proteasomal degradation, thereby blocking Smad2/3 activation, facilitating receptor degradation, and eventually terminating Smad-mediated signaling.

The terms “subject”, “animal,” and “patient” are used interchangeably to refer, for example, and without limitation, to humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals. According to some embodiments, the terms “animal,” “patient,” and “subject” may refer to any or combination of: dogs, cats, pigs, cows, horses, goats, sheep or other domesticated non-human mammals.

The term a “subject in need” of treatment for a particular condition can be a subject having that condition, diagnosed as having that condition, or at risk of developing that condition.

The term “T cell” or T lymphocyte,” as used herein, refers to one of the two types of antigen-specific lymphocytes responsible for adaptive immune responses, the other being the B cells. T cells are responsible for cell-mediated adaptive immune reactions. The highly variable antigen receptor on T cells is called the T cell receptor, which recognizes a complex of primarily peptide antigen bound to major histocompatibility complex (MHC) molecules on cell surfaces. There are two main lineages of T cells: those carrying α:β receptors, and those carrying γ:δ receptors. Effector T cells perform a variety of functions in immune responses, acting always by interacting with another cell in an antigen-specific manner. Some T cells activate macrophages, some help B cells produce antibody, and some kill cells infected with viruses and other intracellular pathogens.

The term “T-cell receptor” or “TCR,” as used herein, refers to the cell surface receptor for antigen on T lymphocytes. It consists of a disulfide-linked heterodimer of the highly variable α and β chains in a complex with the invariant CD3 and ζ proteins, which have a signaling function. An alternative TCR is made up of variable γ and δ chains in a complex with the invariant CD3 and ζ protein is expressed on a subset of T cells.

The term “therapeutic agent,” as used herein, refers to a drug, molecule, composition or other substance that provides a therapeutic effect. The term “active,” as used herein, refers to the ingredient, component or constituent of the compositions of the present invention responsible for the intended therapeutic effect. The terms “therapeutic agent” and “active agent” are used interchangeably.

The term “therapeutic component,” as used herein, refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The term “therapeutic effect,” as used herein, refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

Therapeutic window, potency and efficacy. The term “potency,” as used herein, refers to efficacy, effectiveness, or strength of a drug. The potency of a drug is the reciprocal of dose, and has the units of persons/unit weight of drug or body weight/unit weight of drug. Relative potency compares the relative activity of drugs in a series relative to some prototypic member of the series. “Efficacy” connotes the property of a drug to achieve the desired response, and maximum efficacy denotes the maximum achievable effect.

The intensity of effect of a drug (y-axis) can be plotted as a function of the dose of drug administered (X-axis). Goodman & Gilman's The Pharmacological Basis of Therapeutics, Ed. Joel G. Hardman, Lee E. Limbird, Eds., 10th Ed., McGraw Hill, New York (2001), p. 25, 50). These plots are referred to as dose-effect curves. Such a curve can be resolved into simpler curves for each of its components. These concentration-effect relationships can be viewed as having four characteristic variables: potency, slope, maximal efficacy, and individual variation.

The location of the dose-effect curve along the concentration axis is an expression of the potency of a drug. Id. If the drug is to be administered by transdermal absorption, a highly potent drug is required, since the capacity of the skin to absorb drugs is limited.

The slope of the dose-effect curve reflects the mechanism of action of a drug. The steepness of the curve dictates the range of doses useful for achieving a clinical effect.

Maximal or clinical efficacy refers to the maximal effect that can be produced by a drug. Maximal efficacy is determined principally by the properties of the drug and its receptor-effector system and is reflected in the plateau of the curve. In clinical use, a drug's dosage may be limited by undesired effects.

Biological variability. An effect of varying intensity may occur in different individuals at a specified concentration or a drug. It follows that a range of concentrations may be required to produce an effect of specified intensity in all subjects.

Lastly, different individuals may vary in the magnitude of their response to the same concentration of a drug when the appropriate correction has been made for differences in potency, maximal efficacy and slope.

The duration of a drug's action is determined by the time period over which concentrations exceed the MEC. Following administration of a dose of drug, its effects usually show a characteristic temporal pattern. A plot of drug effect vs. time illustrates the temporal characteristics of drug effect and its relationship to the therapeutic window. A lag period is present before the drug concentration exceeds the minimum effective concentration (MEC) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. The therapeutic window reflects a concentration range that provides efficacy without unacceptable toxicity. Accordingly, another dose of drug should be given to maintain concentrations within the therapeutic window.

As used herein, the term “tissue” refers to a collection of similar cells and the intercellular substances surrounding them.

The term “transcription factor,” as used herein, refers to proteins that bind to DNA-regulatory sequences (enhancers and silencers), usually localized in the 5′-upstream region of target genes, to modulate the rate of gene transcription. This may result in increased or decreased gene transcription, protein synthesis, and subsequent altered cellular function. Examples include, without limitation, Forkhead Box F1 (FOXF1), which during development promotes lung morphogenesis by regulating mesenchymal-epithelial signaling and stimulating cellular proliferation in fetal lung mesenchyme; CREB3 Regulatory Factor (CREBRF), which acts as a negative regulator of the endoplasmic reticulum stress response or unfolded protein response (UPR); it represses the transcriptional activity of CREB3 during the UPR; TSC22 Domain Family Member 1 (TSC22D1), which encodes a member of the TSC22 domain family of leucine zipper transcription factors; the encoded protein is stimulated by transforming growth factor beta, and regulates the transcription of multiple genes; MAX Interactor 1, Dimerization Protein (MXI1), which is a transcriptional repressor thought to negatively regulate MYC function; Kruppel Like Factor 9 (KLF9), a transcription factor that binds to GC box elements located in the promoter; binding of the encoded protein to a single GC box inhibits mRNA expression while binding to tandemly repeated GC box elements activates transcription; Nuclear factor erythroid 2-related factor 2 (NFE2L2), a transcription factor that plays a key role in the response to oxidative stress, High-mobility group AT-hook 2 (HMGA2), a non-histone architectural transcription factor that modulates the transcription of several genes by binding to AT-rich sequences in the minor groove of B-form DNA and alters the chromatin structure; and Double PHD Fingers 3 (DPF3), which belongs to the neuron-specific chromatin remodeling complex (nBAF complex; in the complex, it acts as a tissue-specific anchor between histone acetylations and methylations and chromatin remodeling.

The term “transcriptome,” as used herein, refers to the full range of messenger RNA (or mRNA) molecules expressed by an organism. The term “transcriptome” also refers to the array of mRNA transcripts produced in a particular cell or tissue type.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

Treatment includes eliciting a clinically significant response without excessive levels of side effects. Treatment also includes prolonging survival as compared to expected survival if not receiving treatment.

The term “wound healing” refers to the processes by which the body repairs trauma to any of its tissues, especially those caused by physical means and with interruption of continuity. A wound-healing response can be viewed as comprising four separate phases, comprising: 1) an initial phase post injury involving hemostasis; 2) a second phase involving inflammation; 3) a third phase involving granulation and proliferation; and 4) a fourth phase involving remodeling and maturation. The culmination of the wound-healing response results in the replacement of normal tissue structures with fibroblastic mediated scar tissue. Processes involved in the wound healing response, however, can go awry and produce an exuberance of fibroblastic proliferation, which can result in tissue damage, including hypertrophic scarring (a widened or unsightly scar that does not extend the original boundaries of the wound).

Initial Phase—Hemostatsis

An initial injury results in an outflow of blood and lymphatic fluid. This is also the process during which the initial reparative blood clot is created. Both the intrinsic coagulation pathways, so called because all of the components are intrinsic to plasma, and the extrinsic coagulation pathways are activated. The intrinsic and extrinsic systems converge to activate the final common pathways causing fibrin formation. It is generally recognized that these systems function together and interact in vivo.

The intrinsic coagulation pathway is initiated when blood contacts any surface except normal endothelial and blood cells. This pathway, also known as the contact activation pathway, begins with formation of the primary complex on collagen by high-molecular weight kininogen (HMWK), prekallikrein, and coagulation factor (Factor) XII (Hageman factor). Prekallikrein is converted to kallikrein and Factor XII becomes Factor XIIa. Factor XIIa converts Factor XI into Factor XIa. Factor XIa activates Factor IX, which, with its co-factor FVIIIa form the tenase complex, which activates Factor X to Factor Xa.

The extrinsic coagulation pathway, also known as the tissue factor pathway, generates a thrombin burst and is initiated when tissue thromboplastin activates Factor VII. Upon vessel injury, tissue factor (TF), a nonenzymatic lipoprotein cofactor that greatly increases the proteolytic efficiency of Factor Vila, is exposed to the blood and enzyme coagulation factor VII (proconvertin) circulating in the blood. Once bound to TF, Factor VII is activated to Factor Vila by different proteases, including thrombin (Factor IIa), Factors Xa, IXa, XIIa and the Factor VIIa-TF complex itself. The Factor VIIa-TF complex activates Factors IX and X. The activation of Factor Xa by the Factor VIIa-TF complex almost immediately is inhibited by tissue factor pathway inhibitor (TFPI). Factor Xa and its cofactor Va form the prothrombinase complex which activates the conversion of prothrombin to thrombin. Thrombin then activates other components of the coagulation cascade, including Factors V and VIII (which activates Factor XI, which, in turn, activates Factor IX), and activates and releases Factor VIII from being bound to von Willebrand Factor (vWF). Factors Vila and IXa together form the “tenase” complex, which activates Factor X, and so the cycle continues.

As currently understood, coagulation in vivo is a 3-step process centered on cell surfaces. In the first step, coagulation begins primarily by initiation with tissue factor, which is present on the subendothelium, tissues not normally exposed to blood, activated monocytes and endothelium when activated by inflammation. Factors VII and Vila bind to tissue factor and adjacent collagen. The factor Vila-tissue factor complex activates factor X and IX. Factor Xa activates factor V, forming a prothrombinase complex (factor Xa, Va and calcium) on the tissue factor expressing cell. In the second step, coagulation is amplified as platelets adhere to the site of injury in the blood vessel. Thrombin is activated by platelet adherence and then acts to fully activate platelets, enhance their adhesion and to release factor V from the platelet α-granules. Thrombin on the surface of activated platelets activates factors V, VIII and XI, with subsequent activation of factor IX. The tenase complex (factors IXa, Villa and calcium) now is present on platelets where factor Xa can be produced and can generate another prothrombinase complex on the platelet so that there can be large-scale production of thrombin. Propagation, the third step, and is a combination of activation of the prothrombinase complexes that allow large amounts of thrombin to be generated from prothrombin. More platelets can be recruited, as well as activation of fibrin polymers and factor XIII.

The inflammatory phase (see below) begins during the hemostasis phase. Thrombocytes, as well as recruited white blood cells, release numerous factors to ramp up the healing process. Alpha-granules liberate platelet-derived growth factor (PDGF), platelet factor IV, and transforming growth factor beta (TGF-β). The processes of inflammation, collagen degradation and collagenogenesis, myoblastic creation from transformed fibroblasts, growth of new blood vessels, and reepithelialization are mediated by a host of cytokines and growth factors. The interleukins strongly influence the inflammatory process. Vascular endothelial growth factor (VEGF) and other factors enhance blood vessel formation, and some have multiple roles, such as fibroblast growth factor (FGF)-2, which affects not only the process of angiogenesis but also that of reepithelialization. Vasoactive amines, such as histamine and serotonin, are released from dense bodies found in thrombocytes. PDGF is chemotactic for fibroblasts and, along with TGF-β, is a potent modulator of fibroblastic mitosis, leading to prolific collagen fibril construction in later phases. Fibrinogen is cleaved into fibrin, and the framework for completion of the coagulation process is formed. Fibrin provides the structural support for cellular constituents of inflammation. This process starts immediately after the insult and may continue for a few days.

Second Phase: Inflammation

The early component of the inflammatory phase is predominated by the influx of the polymorphonuclear leukocytes (PMNs) and the later component of the inflammatory phase is predominated by monocytes/macrophages.

Within the first 6-8 hours, PMNs engorge the wound. TGF-β facilitates PMN migration from surrounding blood vessels, from which they extrude themselves from these vessels. These cells cleanse the wound, clearing it of debris. The PMNs attain their maximal numbers in 24-48 hours and commence their departure by hour 72. Other chemotactic agents are released, including FGF, TGF-β and TGF-α, PDGF, and plasma-activated complements C3a and C5a (anaphylactic toxins). They are sequestered by macrophages or interred within the scab or eschar.

As the process continues, monocytes also exude from surrounding blood vessels. Once they leave the vessel, these are termed macrophages. The macrophages continue the cleansing process, manufacture various growth factors during days 3-4, and orchestrate the multiplication of endothelial cells with the sprouting of new blood vessels, the duplication of smooth muscle cells, and the creation of the milieu created by the fibroblast. Many factors influencing the wound healing process are secreted by macrophages, including TGFs, cytokines and interleukin (IL), tumor necrosis factor (TNF), and PDGF.

Third Phase: Granulation and Proliferation

The granulation and proliferation phase consists of an overall and ongoing process, comprising subphases termed the “fibroplasia, matrix deposition, angiogenesis and reepithelialization” subphases.

By days 5-7, fibroblasts have migrated into the wound, laying down new collagen of subtypes I and III. Early in normal wound healing, type III collagen predominates but is later replaced by type I collagen.

Tropocollagen is the precursor of all collagen types and is transformed within the cell's rough endoplasmic reticulum, where proline and lysine are hydroxylated. Disulfide bonds are established, allowing 3 tropocollagen strands to form a triple left-handed triple helix, termed procollagen. As the procollagen is secreted into the extracellular space, peptidases in the cell membrane cleave terminal peptide chains, creating true collagen fibrils.

The wound is suffused with glycosaminoglycans (GAGs) and fibronectin produced by fibroblasts. These GAGs include heparin sulfate, hyaluronic acid, chondroitin sulfate, and keratin sulfate. Proteoglycans are GAGs that are bonded covalently to a protein core and contribute to matrix deposition.

Angiogenesis results from parent vessel offshoots. The formation of new vasculature requires extracellular matrix and basement membrane degradation followed by migration, mitosis, and maturation of endothelial cells. Basic FGF and vascular endothelial growth factor are believed to modulate angiogenesis.

Re-epithelization occurs with the migration of cells from the periphery of the wound and accessory or adjoining tissues. This process commences with the spreading of cells within 24 hours. Division of peripheral cells occurs in hours 48-72, resulting in a thin epithelial cell layer, which bridges the wound. Epidermal growth factors are believed to play a key role in this aspect of wound healing.

This succession of subphases can last up to 4 weeks in the clean and uncontaminated wound.

Fourth Phase: Remodeling and Maturation

After the third week, the wound undergoes constant alterations, known as remodeling, which can last for years after the initial injury occurred. Collagen is degraded and deposited in an equilibrium-producing fashion, resulting in no change in the amount of collagen present in the wound. The collagen deposition in normal wound healing reaches a peak by the third week after the wound is created. Contraction of the wound is an ongoing process resulting in part from the proliferation of specialized fibroblasts termed myofibroblasts, which provide mechanical support and integrity to the tissue after initial injury. Wound contraction occurs to a greater extent with secondary healing (i.e., healing by second intention, which describes a wound left open and allowed to close by reepithelialization and contraction by myofibroblasts) than with primary healing (i.e., healing by first intention, which describes a wound closed by approximation of wound margins or by placement of a graft or flap, or wounds created and closed in the operating room, unlike via reepithelialization and contraction by myofibroblasts). Maximal tensile strength (the greatest longitudinal stress a substance can bear without tearing apart) of the wound is achieved by the 12th week, and the ultimate resultant scar has only 80% of the tensile strength of the original skin that it has replaced. At the end of tissue repair, the reconstructed ECM takes over the mechanical load and myofibroblasts disappear by massive apoptosis.

### Embodiments

To define the molecular mechanisms of fibroblast invasion, we performed a single cell RNA-seq survey of invasive fibroblasts and non-invasive fibroblasts from four normal and four IPF human lungs using an in vitro assay system which had been previously used to evaluate the ability of lung fibroblasts to spontaneously invade Matrigel and commonly used to analyze the metastatic potential of cancer cells. Different subtypes in invasive or non-invasive lung fibroblasts were classified, and their gene signatures, specific cell surface markers, long non-coding RNA (lncRNA), key transcription factors and signaling pathways were further confirmed mechanistically and functionally. After combination of all the specific genes for Ingenuity Pathway Analysis (IPA), we found that the ERBB2 (HER2) signaling pathway was significantly activated in invasive fibroblasts and was markedly inhibited in non-invasive fibroblasts. Blocking HER2 signaling by a small-molecular inhibitor or a neutralizing antibody inhibited fibroblast invasion and lung fibrosis.

### Methods

According to one aspect, the present disclosure provides a method for treating a subject with a progressive lung disease comprising administering a pharmaceutical composition comprising a Human Epidermal Growth Factor Receptor 2 (HER2) blocking agent and a pharmaceutically acceptable carrier, wherein the method improves clinical outcome compared to an untreated control. HER2 is a transmembrane tyrosine kinase receptor which belongs to the family of the EGFR (epidermal growth factor receptor).

In various embodiments, the subject does not have cancer. In various embodiments, the method further includes identifying the subject in need of pulmonary fibrosis treatment before administering the pharmaceutical composition including the HER2 blocking agent.

According to some embodiments, the progressive lung disease is a fibrotic lung disease. According to some embodiments, the fibrotic lung disease is pulmonary fibrosis. According to some embodiments, the pulmonary fibrosis is idiopathic pulmonary fibrosis (IPF).

According to some embodiments of the method, the HER2 blocking agent comprises an anti-HER2 antibody, a small molecule HER2 inhibitor, or both.

According to some embodiments, the anti-HER2 antibody comprises trastuzumab, pertuzumab, inetetamab; or margutuximab or a biosimilar thereof.

According to some embodiments, the anti-HER2 antibody comprises trastuzumab (sold under the brand name of Herceptin®). Trastuzumab is a humanized mAb directed against the extracellular domain of the tyrosine kinase receptor HER2 (also known as HER2/neu). According to some embodiments, the anti-HER2 antibody is a biosimilar of Herceptin, e.g., trastuzumab-pkrb (sold as Herzuma), trastuzumab-anns (sold as Kanjinti), trastuzumab-dkst (sold as Ogiviri), trastuzumab-dttb (sold as Ontruzant), trastuzumab-qyyp (sold as Traziimera). According to some embodiments, the anti-HER2 antibody comprises margetuximab-cmkb (sold as Margenza) a monoclonal antibody derivative of trastuzumab that binds to the same epitope of the HER2 receptor with similar affinity and antiproliferative effects as trastuzumab. In comparison to trastuzumab, it has an IgG1 Fc region that is genetically engineered to have up to 6.6× greater affinity for the stimulatory CD16A FcγRIIIA on NK cells and 8.4× less affinity for inhibitory CD32B FcγRIIB found on immune effector cells (NK cells and macrophages) within the innate immune system's antibody dependent cytotoxicity (ADCC) process.

According to some embodiments, the anti-HER2 antibody comprises pertuzumab (sold as Perjeta). Pertuzumab is a humanized monoclonal antibody that binds to the extracellular domain II of HER2. Because domain II of HER2 is a region necessary for dimerization with other HER family receptors and signaling, pertuzumab inhibits ligand-induced dimerization and its downstream signaling. Its mechanism of action is complementary to trastuzumab (sold under the brand name of Herceptin®), inhibiting ligand-dependent HER2-HER3 dimerization and reducing signaling via intracellular pathways such as phosphatidylinositol 3-kinase (PI3K/Akt). According to some embodiments, the anti-HER2 antibody comprises trastuzumab and pertuzumab.

According to some embodiments, the small molecule is lapatinib, a small molecule tyrosine kinase inhibitor of both the epidermal growth factor receptor (EGFR) and HER2. Lapatinib ditosylate (GW2016; GW572016; TYKERB®), a member of the quinazoline family with a 4-anilinoquinazoline core, is a reversible, small molecule tyrosine kinase dual inhibitor of EGFR and HER2. In contrast to trastuzumab which binds to the extracellular domain of HER2, lapatinib exerts its activity intracellularly by competing with ATP for the ATP-binding domain in the cytoplasmic tail of the tyrosine kinase receptor. Inhibition of tyrosine kinase phosphorylation is its major mechanism of action, which dampens and/or abrogates signal transduction along the PI3K/Akt and the ras/raf/MAPK pathways. Compared with erlotinib and gefitinib (two isolated EGFR-specific tyrosine kinase inhibitors), lapatinib binds to an inactive-like conformation of EGFR and has comparatively slower inhibitor dissociation rates, with estimated Ki-app values of 3 nM and 13 nM against EGFR and ErbB2 respectively.

According to some embodiments, the small molecule is Neratinib (Nerlynx®), an irreversible oral pan-TK (tyrosine kinase) inhibitor of several members of the HER family (EGFR/IHER1, HER2, HER4).

According to some embodiments, the small molecule inhibitor comprises tucatinib (ONT-380), which binds to and inhibits the intracellular TK domain of the HER2 receptor. Tucatinib inhibits pro-proliferative downstream cell-signaling pathways of HER2 receptor activation and demonstrates promise crossing the blood-brain-barrier.

According to some embodiments, the small molecule inhibitor comprises Pyrotinib, an irreversible pan-TK inhibitor of the HER family (HER1, HER2, HER4) of transmembrane TK-receptors. It works by inhibiting downstream pro-proliferative signaling.

According to some embodiments, the small molecule inhibitor comprises dacomitinib (VIZIMPRO®, Pfizer), an irreversible small molecule inhibitor of the activity of the human epidermal growth factor receptor (EGFR) family (EGFR/IER1, HER2, and HER4) tyrosine kinases. It achieves irreversible inhibition via covalent bonding to the cysteine residues in the catalytic domains of the HER receptors.

According to some embodiments, the small molecule inhibitor comprises afatinib maleate (GILOTRIF®, Boehringer Ingelheim Pharma GmbH & Co. KG)). Afatinib is a protein kinase inhibitor that also irreversibly inhibits human epidermal growth factor receptor 2 (Her2) and epidermal growth factor receptor (EGFR) kinases.

According to some embodiments, the HER2 blocking agent comprises a recombinant hyaluronidase to facilitate delivery. According to some embodiments, the anti-HER2 antibody comprises trastuzumab, pertuzumab and hyaluronidase-zzxf (sold as Phesgo).

According to some embodiments, the HER2 blocking agent is an antibody drug conjugate. For example, according to some embodiments, the anti-HER2-antibody comprises trastuzumab emtansine (sold as Kadcyla or T-DM1). T-DM1 is an antibody-drug conjugate, which has several mechanisms of action consisting of the anti-HER2 effects of trastuzumab and the effects of DM1, a cytotoxic anti-microtubule agent released within the target cells upon degradation of the human epidermal growth factor receptor-2 (HER2)-T-DM1 complex in lysosomes. According to some embodiments, the anti-HER2 antibody comprises a trastuzumab drug conjugate comprising fam-trastuzumab deruxecan-nxki (sold as Enhertu® or DS-8201a), a humanized anti-HER2 IgG1 with a small molecule, DXd, which is a topoisomerase I inhibitor, attached to the antibody by a cleavable linker. According to some embodiments, the anti-HER2 antibody comprises disitamab vedotin in which the antibody component is a humanized monoclonal antibody targeting HER2, and the small molecule toxin is monomethyl auristatin E (MMAE), a synthetic antineoplastic agent with a protease cleavable linker covalently attached MMAE to the antibody. Disitamab vedotin selectively delivers anti-cancer agent monomethyl auristatin E (MMAE) into HER2+ tumor cells.

According to some embodiments, the clinical outcome is progression free survival. According to some embodiments, the clinical outcome is overall survival.

According to some embodiments, the method decreases one or more symptoms of the fibrotic lung disease. For example, the symptoms of the fibrotic lung disease include shortness of breath (dyspnea), a dry cough, fatigue, unexplained weight loss, aching muscles and joints, and widening and rounding of the tips of the fingers or toes (clubbing).

According to some embodiments, the method increases repair of a lung injury. According to some embodiments, the method slows progression of fibrotic lung disease. According to some embodiments, the method, increases renewal of alveolar epithelial cell 2 (AEC2) cells. According to some embodiments, compared to an untreated control, the method decreases migration, invasion or both by lung fibroblasts obtained from the subject.

According to some embodiments, the present disclosure provides a method for treating a subject with a progressive lung disease with a pharmaceutical composition comprising a HER2 blocking agent, the method including: (a) identifying or having identified the subject as one who can benefit therapeutically from being treated with the pharmaceutical composition by: (1) obtaining or having obtained a biological sample from the subject, wherein the biological sample comprises non-invasive lung fibroblasts, invasive lung fibroblasts, or both; and (2) determining or having determined an RNA profile for the biological sample by: (i) identifying and quantifying or having identified and quantified expression of RNA from the lung fibroblasts in the biological sample by RNA sequencing; and (ii) comparing or having compared the RNA profile for the lung fibroblasts in the biological sample to an RNA profile of lung fibroblasts obtained from a healthy control subject; (b) identifying or having identified the subject as one who can benefit therapeutically from being treated with the pharmaceutical composition comprising the HER2 blocking agent, when the RNA profile from the biological sample is distinguishable from the RNA profile from the healthy control subject; and (c) treating the identified subject with the pharmaceutical composition comprising the HER2 blocking agent, wherein the treating improves clinical outcome.

According to further embodiments, the subject is identified by determining or having determined an index of migration for the lung fibroblasts in the biological sample from the subject by: (i) in a 3D in vitro migration assay, quantifying or having quantified the lung fibroblasts that have migrated through a porous membrane and a matrix comprising extracellular matrix proteins towards a fibroblast chemoattractant after about 24 hours; and (ii) normalizing or having normalized a count of the lung fibroblasts in (i) to a number of lung fibroblasts obtained from a control subject that have migrated through the membrane and matrix obtained from a control subject, thereby determining the index of migration; and identifying or having identified the subject as one who can benefit therapeutically from being treated with the pharmaceutical composition comprising the HER2 blocking agent, when (i) the index of migration is about 1.5 fold, about 2 fold, about 2.5 fold, about 3 fold, about 3.5 fold, or about 4 fold higher than an index of migration of fibroblasts derived from a control, and/or (ii) the index of migration is about 1.5 fold, about 2 fold, about 2.5 fold, about 3 fold, about 3.5 fold, or about 4 fold lower than an index of migration of fibroblasts derived from a control.

According to further embodiments, the subject is identified by determining or having determined an index of invasion for the lung fibroblasts derived from the biological sample from the subject by: (i) in a 3D in vitro invasion assay, quantifying or having quantified the lung fibroblasts derived from the biological sample from the subject that have migrated through a porous membrane and into a matrigel matrix towards a fibroblast chemoattractant after about 24 hours; and (ii) normalizing or having normalized a count of the lung fibroblasts in (i) to a number of lung fibroblasts obtained from a control subject that have migrated through a porous membrane and into a matrigel matrix towards the fibroblast chemoattractant, thereby determining the index of invasion; and (b) identifying or having identified the subject as one who can benefit therapeutically from being treated with the pharmaceutical composition comprising the HER2 blocking agent.

According to some embodiments, the present disclosure provides a method for identifying a subject with a progressive lung disease as one who can benefit therapeutically from being treated with a pharmaceutical composition comprising a HER2 blocking agent, the method including: (1) obtaining a biological sample from the subject, wherein the biological sample comprises non-invasive lung fibroblasts, invasive lung fibroblasts, or both; and (2) determining an RNA profile for the biological sample by: (i) identifying and quantifying expression of RNA from the lung fibroblasts in the biological sample by RNA sequencing; and (ii) comparing the RNA profile for the lung fibroblasts in the biological sample to an RNA profile of lung fibroblasts obtained from a healthy control subject. The subject is identified as the one who can benefit therapeutically from being treated with the pharmaceutical composition comprising the HER2 blocking agent, when the RNA profile from the biological sample is distinguishable from the RNA profile from the healthy control subject,

According to some embodiments, a method for identifying a subject with a progressive lung disease as one who can benefit therapeutically from being treated with a pharmaceutical composition comprising a HER2 blocking agent includes determining an index of migration for the lung fibroblasts in the biological sample from the subject by: (i) in a 3D in vitro migration assay, quantifying the lung fibroblasts that have migrated through a porous membrane and a matrix comprising extracellular matrix proteins towards a fibroblast chemoattractant after about 24 hours; and (ii) normalizing a count of the lung fibroblasts in (i) to a number of lung fibroblasts obtained from a control subject that have migrated through the membrane and matrix obtained from a control subject, thereby determining the index of migration. The subject is identified as the one who can benefit therapeutically from being treated with the pharmaceutical composition comprising the HER2 blocking agent, when the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold higher than an index of migration of fibroblasts derived from a control, and/or (iii) the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold lower than an index of migration of fibroblasts derived from a control.

According to some embodiments, a method for identifying a subject with a progressive lung disease as one who can benefit therapeutically from being treated with a pharmaceutical composition comprising a HER2 blocking agent includes determining an index of invasion for the lung fibroblasts derived from the biological sample from the subject by: (i) in a 3D in vitro invasion assay, quantifying the lung fibroblasts derived from the biological sample from the subject that have migrated through a porous membrane and into a matrigel matrix towards a fibroblast chemoattractant after about 24 hours; and (ii) normalizing a count of the lung fibroblasts in (i) to a number of lung fibroblasts obtained from a control subject that have migrated through a porous membrane and into a matrigel matrix towards the fibroblast chemoattractant, thereby determining the index of invasion.

According to some embodiments, the present disclosure provides a method for treating a subject with a progressive lung disease and identified as one who can benefit therapeutically from being treated with a pharmaceutical composition comprising a HER2 blocking agent, the method including treating the identified subject with the pharmaceutical composition comprising the HER2 blocking agent such that clinical outcome improves by the treating, wherein the subject is identified as the one who can benefit therapeutically from being treated with the pharmaceutical composition by: (1) obtaining or having obtained a biological sample from the subject, wherein the biological sample comprises non-invasive lung fibroblasts, invasive lung fibroblasts, or both; (2) determining or having determined an RNA profile for the biological sample by: (i) identifying and quantifying or having identified and quantified expression of RNA from the lung fibroblasts in the biological sample by RNA sequencing; and (ii) comparing or having compared the RNA profile for the lung fibroblasts in the biological sample to an RNA profile of lung fibroblasts obtained from a healthy control subject. The subject is identified as the one who can benefit therapeutically from being treated with the pharmaceutical composition comprising the HER2 blocking agent, when (i) the RNA profile from the biological sample is distinguishable from the RNA profile from the healthy control subject

According to further embodiments, a method for treating a subject with a progressive lung disease and identified as one who can benefit therapeutically from being treated with a pharmaceutical composition comprising a HER2 blocking agent includes determining or having determined an index of migration for the lung fibroblasts in the biological sample from the subject by: (i) in a 3D in vitro migration assay, quantifying or having quantified the lung fibroblasts that have migrated through a porous membrane and a matrix comprising extracellular matrix proteins towards a fibroblast chemoattractant after about 24 hours; and (ii) normalizing or having normalized a count of the lung fibroblasts in (i) to a number of lung fibroblasts obtained from a control subject that have migrated through the membrane and matrix obtained from a control subject, thereby determining the index of migration. The subject is identified as the one who can benefit therapeutically from being treated with the pharmaceutical composition comprising the HER2 blocking agent, when (ii) the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold higher than an index of migration of fibroblasts derived from a control, and/or (iii) the index of migration is about 1.5 fold, about 2 fold, about 2.5 fold, about 3 fold, about 3.5 fold, about 4 fold, about 1.5 fold to about 4 fold, about 1.5 fold to about 3 fold, or about 3 fold to about 4 fold lower than an index of migration of fibroblasts derived from a control.

According to further embodiments, a method for treating a subject with a progressive lung disease and identified as one who can benefit therapeutically from being treated with a pharmaceutical composition comprising a HER2 blocking agent includes determining or having determined an index of invasion for the lung fibroblasts derived from the biological sample from the subject by: (i) in a 3D in vitro invasion assay, quantifying or having quantified the lung fibroblasts derived from the biological sample from the subject that have migrated through a porous membrane and into a matrigel matrix towards a fibroblast chemoattractant after about 24 hours; and (ii) normalizing or having normalized a count of the lung fibroblasts in (i) to a number of lung fibroblasts obtained from a control subject that have migrated through a porous membrane and into a matrigel matrix towards the fibroblast chemoattractant, thereby determining the index of invasion.

According to some embodiments, the subject is identified as the one who can benefit therapeutically from being treated with the pharmaceutical composition comprising the HER2 blocking agent, when:
CD108 (SEMA7A) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to RNA from lung fibroblasts of the normal healthy control subject;
CD142 (F3) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject;
CD49F (ITGA6) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject;
PD-L1 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or
PD-L2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when:
transcription factor FOXF1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject;
transcription factor CREBRF encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject;
transcription factor TSC22D1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject;
transcription factor MXI1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject;
transcription factor NFE2L2 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or
transcription factor KLF9 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when:
transcription factor HMGA2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject; and/or
transcription factor DPF3 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the RNA from lung fibroblasts of the normal healthy control subject.

According to some embodiments, the subject is identified as the one who can benefit therapeutically from being treated with the pharmaceutical composition comprising the HER2 blocking agent, when regulation of lncRNA from the subject, which comprises one or more of LINC00152 and FENDRR, is different compared to regulation of lncRNA from the normal healthy control subject such that:
the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject; and/or
the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the normal healthy control subject.

According to some embodiments, the biological sample comprises lung tissue. According to some embodiments, the biological sample comprises bronchoalveolar lavage fluid.

According to some embodiments, the membrane has a pore size of about 0.8 μm. According to some embodiments, the chemoattractant is a growth factor. According to some embodiments, the growth factor is platelet-derived growth factor (PDGF).

According to some embodiments, the identified and quantified RNA from the lung fibroblasts derived from the subject is an RNA that encodes cell surface markers, transcription factors, long non-coding RNA (lncRNA), or a combination thereof.

According to some embodiments, the cell surface markers encoded by the RNA from the subject include one or more of CD108 (SEMA7A), CD142 (F3), CD49F (ITGA6), PD-L1, and PD-L2. According to some embodiments, the cell surface markers encoded by the RNA from the subject include two or more of CD108 (SEMA7A), CD142 (F3), CD49F (ITGA6), PD-L1, and PD-L2. According to some embodiments, the cell surface markers encoded by the RNA from the subject include three or more of CD108 (SEA7A), CD142 (F3), CD49F (ITGA6), PD-L1, and PD-L2. According to some embodiments, the cell surface markers encoded by the RNA from the subject include four or more of CD108 (SEMA7A), CD142 (F3), CD49F (ITGA6), PD-L1, and PD-L2. According to some embodiments, the cell surface markers encoded by the RNA from the subject include CD108 (SEMA7A), CD142 (F3), CD49F (ITGA6), PD-L1, and PD-L2.

According to some embodiments, one or more, two or more, three or more, four or more, or all of CD108 (SEMA7A), CD142 (F3), CD49F (ITGA6), PD-L1, and PD-L2 encoded by the RNA from the subject is upregulated compared to the RNA from the normal healthy control. According to some embodiments, the cell surface marker CD108 (SEMA7A) encoded by the RNA from the subject is upregulated about 1.5 fold, about 2 fold, about 2.5 fold, about 3 fold, about 3.5 fold, about 4 fold, about 4.5 fold, or about 5 fold compared to the RNA encoded by healthy control. According to some embodiments, the cell surface marker CD142 (F3) encoded by the RNA from the subject is upregulated about 1.5 fold, about 2 fold, about 2.5 fold, about 3 fold, about 3.5 fold, about 4 fold, about 4.5 fold, or about 5 fold compared to the RNA from the healthy control. According to some embodiments, the cell surface marker CD49F (ITGA6) encoded by the RNA from the subject is upregulated about 1.5 fold, about 2 fold, about 2.5 fold, about 3 fold, about 3.5 fold, about 4 fold, about 4.5 fold, or about 5 fold compared to the RNA from the healthy control. According to some embodiments, the cell surface marker PD-L1 encoded by the RNA from the subject is upregulated about 1.5 fold, about 2 fold, about 2.5 fold, about 3 fold, about 3.5 fold, about 4 fold, about 4.5 fold, or about 5 fold compared to the RNA from the healthy control. According to some embodiments, the cell surface marker PD-L2 encoded by the RNA from the subject is upregulated about 1.5 fold, about 2 fold, about 2.5 fold, about 3 fold, about 3.5 fold, about 4 fold, about 4.5 fold, or about 5 fold compared to the RNA from the healthy control.

According to some embodiments, the transcription factors encoded by the RNA from the subject include one or more of HMGA2, DPF3, FOXF1, CREBRF, TSC22D1, MXI1, NFE2L2, and KLF9. According to some embodiments, the transcription factors encoded by the RNA from the subject include two or more of HMGA2, DPF3, FOXF1, CREBRF, TSC22D1, MXI1, NFE2L2, and KLF9. According to some embodiments, the transcription factors encoded by the RNA from the subject include three or more of HMGA2, DPF3, FOXF1, CREBRF, TSC22D1, MXI, NFE2L2, and KLF9. According to some embodiments, the transcription factors encoded by the RNA from the subject include four or more of HMGA2, DPF3, FOXF1, CREBRF, TSC22D1, MXI1, NFE2L2, and KLF9. According to some embodiments, the transcription factors encoded by the RNA from the subject include five or more of HMGA2, DPF3, FOXF1, CREBRF, TSC22D1, MXI1, NFE2L2, and KLF9. According to some embodiments, the transcription factors encoded by the RNA from the subject include six or more of HMGA2, DPF3, FOXF1, CREBRF, TSC22D1, MXI1, NFE2L2, and KLF9. According to some embodiments, the transcription factors encoded by the RNA from the subject include seven or more of HMGA2, DPF3, FOXF1, CREBRF, TSC22D1, MXI1, NFE2L2, and KLF9. According to some embodiments, the transcription factors encoded by the RNA from the subject include HMGA2, DPF3, FOXF1, CREBRF, TSC22D1, MXI1, NFE2L2, and KLF9.

According to some embodiments, the transcription factors FOXF1, CREBRF, TSC22D1, MXI1, NFE2L2, and KLF9 encoded by the RNA from the subject are downregulated. According to some embodiments, the transcription factor FOXF1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the transcription factor CREBRF encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the transcription factor TSC22D1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the transcription factor MXI1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the transcription factor NFE2L2 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the transcription factor KLF9 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control.

According to some embodiments, the transcription factors HMGA2 and DPF3 encoded by the RNA from the subject are upregulated compared to the healthy control. According to some embodiments, the transcription factor HMGA2 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the transcription factor DPF3 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control.

According to some embodiments, the lncRNA from the subject comprises one or more of LINC00152 and FENDRR. According to some embodiments, the FENDRR encoded by the lncRNA from the subject is down regulated and LINC00152 is upregulated compared to the healthy control. According to some embodiments, the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control. According to some embodiments, the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to 5 fold compared to the healthy control.

According to some embodiments, the HER2 blocking agent decreases the index of migration by about 1.5 fold, 2 fold, 2.5 fold, or about 1.5 fold to about 2.5 fold compared to an untreated control. According to some embodiments, the HER2 blocking agent decreases the index of invasion by about 1.5 fold, 2 fold, 2.5 fold, or about 1.5 fold to about 2.5 fold compared to an untreated control.

### Compositions

According to some embodiments, the pharmaceutical compositions of the present disclosure comprises one or more active agents. According to some embodiments, the active agent comprises a HER2 blocking agent as described above. According to some embodiments, the active agents include one or a combination of immunomodulators, analgesics, anti-inflammatory compounds, anti-fibrotic compounds, proton pump inhibitors, or a supportive therapy, e.g., oxygen therapy.

Examples of immunomodulators include corticosteroids, for example, prednisone, azathioprine, mycophenolate, mycophenolate mofetil, colchicine, and interferon-gamma 1b.

Examples of analgesics include codeine, hydrocodone, oxycodone, methadone, hydromorphone, morphine, and fentanyl.

Examples of anti-inflammatory compounds include aspirin, celecoxib, diclofenac, diflunisal, etodolac, ibuprofen, indomethacin, ketoprofen, ketorolac nabumetone, naproxen, nintedanib, oxaprozin, pirfenidone, piroxicam, salsalate, sulindac, and tolmetin.

Examples of anti-fibrotic compounds are nintedanib and pirfenidone.

Examples of proton pump inhibitors are omeprazole, lansoprazole, dexlansoprazole, esomeprazole, pantoprazole, rabeprazole, and ilaprazole.

The HER2 blocking agent(s) of the present disclosure may exist in various solid states including an amorphous form (non-crystalline form), and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these forms can be used as an alternative form to the free base or free acid forms of the compounds, and are within the scope of the described invention.

The compounds and compositions of the described invention can be administered, inter alia, as pharmaceutically acceptable salts, esters, amides or prodrugs. The term “salts” is used herein to refer to inorganic and organic salts of compounds of the described invention. The salts can be prepared in situ during the final isolation and purification of a compound, or by separately reacting a purified compound in its free base or acid form with a suitable organic or inorganic base or acid and isolating the salt thus formed.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made. According to some embodiments, the pharmaceutical compositions comprise an HER2 blocking agent, a small molecule (e.g., Lapatinib) or pharmaceutically acceptable salt thereof, or an antibody (e.g., Pertuzumab) or fragment thereof, and at least one pharmaceutically acceptable carrier.

The phrase “pharmaceutically acceptable carrier” is art recognized. It is used to mean any substantially non-toxic carrier conventionally useable for administration of pharmaceuticals in which the isolated polypeptide of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

The compositions may contain wetting agents, emulsifiers and lubricants, such as sodium lauryl sulfate and magnesium stearate, as well as coloring agents, release agents, coating agents, sweetening, flavoring and perfuming agents, preservatives and antioxidants. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil.

The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Formulations of the invention of the present disclosure include those suitable for oral, nasal, topical, buccal, sublingual, rectal, vaginal and/or parenteral administration. The formulations may conveniently be presented in unit dosage form and may be prepared by any methods well known in the art of pharmacy. Specific modes of administration will depend on the indication. The selection of the specific route of administration and the dose regimen is to be adjusted or titrated by the clinician according to methods known to the clinician in order to obtain the optimal clinical response. The amount of compound to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

The amount of active ingredient that can be combined with a carrier material to produce a single dosage form will generally be that amount of the compound that produces a therapeutic effect.

Pharmaceutical formulations containing the compounds of the described invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the described invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The invention of the present disclosure relates to all routes of administration including intramuscular, subcutaneous, sublingual, intravenous, intraperitoneal, intrathecal, intranasal, intratracheal, topical, intradermal, intramucosal, intracavernous, intraocular, intrarectal, into a sinus, gastrointestinal, intraductal, intrathecal, subdural, extradural, intraventricular, intrapulmonary, into an abscess, intraarticular, into a bursa, subpericardial, into an axilla, intrauterine, into the pleural space, intravaginal, intraurethral, intradermal, intrabuccal, transmucosal, transdermal, via inhalation, via nebulizer and via subcutaneous injection. Alternatively, the pharmaceutical composition may be introduced by various means into cells that are removed from the individual. Such means include, for example, microprojectile bombardment and liposome or other nanoparticle device.

The local delivery of therapeutic amounts of a compound for the treatment of a lung injury or fibrotic lung disease can be by a variety of techniques that administer the compound at or near the targeted site. Examples of local delivery techniques are not intended to be limiting but to be illustrative of the techniques available. Examples include local delivery catheters, site specific carriers, implants, direct injection, or direct applications, such as topical application and, for the lungs, administration by inhalation.

Local delivery by an implant describes the surgical placement of a matrix that contains the pharmaceutical agent into the affected site. The implanted matrix releases the pharmaceutical agent by diffusion, chemical reaction, or solvent activators.

For administration by inhalation, the compounds for use according to the described invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compositions or pharmaceutical compositions of the described invention can be formulated for parenteral administration by injection, e.g., by bolus injection or continuous infusion. The compounds can be administered by continuous infusion subcutaneously over a predetermined period of time. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For parenteral administration, a composition or pharmaceutical composition can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of analog in 0.9% sodium chloride solution.

For oral administration, the active agent(s) can be admixed with carriers and diluents, molded into tablets, or enclosed in gelatin capsules. The mixtures can alternatively be dissolved in liquids such as 10% aqueous glucose solution, isotonic saline, sterile water, or the like, and administered intravenously or by injection. The compounds can be formulated readily by combining them with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragecanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Solid dosage forms for oral administration include capsules, tablets, pills, powders and granules. In solid dosage forms, the composition or pharmaceutical compositions are generally admixed with at least one inert pharmaceutically acceptable carrier such as sucrose, lactose, starch, or other generally regarded as safe (GRAS) additives. Such dosage forms can also comprise, as is normal practice, an additional substance other than an inert diluent, e.g., lubricating agent such as magnesium state. With capsules, tablets, and pills, the dosage forms may also comprise a buffering agent. Tablets and pills can additionally be prepared with enteric coatings, or in a controlled release form, using techniques know in the art.

Liquid dosage forms for oral administration include pharmaceutically acceptable emulsions, solutions, suspensions and syrups, with the elixirs containing an inert diluent commonly used in the art, such as water. These compositions can also include one or more adjuvants, such as wetting agent, an emulsifying agent, a suspending agent, a sweetening agent, a flavoring agent or a perfuming agent.

Pharmaceutical preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of, e.g., tablets or lozenges formulated in a conventional manner.

The compounds of the described invention can also be formulated in rectal compositions such as suppositories or retention enemas, e.g., containing conventional suppository bases such as cocoa butter or other glycerides.

In addition to the formulations described previously, the compounds of the described invention can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the described invention, for example, can be applied to a plaster, or can be applied by transdermal, therapeutic systems that are consequently supplied to the organism.

Pharmaceutical compositions comprising one or more active compounds disclosed herein also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

According to the foregoing embodiments, the pharmaceutical composition may be administered once, for a limited period of time or as a maintenance therapy over an extended period of time, for example until the condition is ameliorated, cured or for the life of the subject). A limited period of time may be for 1 week, 2 weeks, 3 weeks, 4 weeks and up to one year, including any period of time between such values, including endpoints. According to some embodiments, the composition or pharmaceutical composition may be administered for about 1 day, for about 3 days, for about 1 week, for about 10 days, for about 2 weeks, for about 18 days, for about 3 weeks, or for any range between any of these values, including endpoints. According to some embodiments, the compound, composition or pharmaceutical composition may be administered for more than one year, for about 2 years, for about 3 years, for about 4 years, or longer.

According to the foregoing embodiments, the compound, composition or pharmaceutical composition may be administered once daily, twice daily, three times daily, four times daily or more to accomplish its therapeutic effect as described herein. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The Examples described herein illustrate several advantages of the described invention and are not intended to limit the scope of what the inventors regard as the invention, nor are they intended to represent that the experiments below are all or the only experiments performed. Efforts have been made to ensure accuracy with respect to numbers used (e.g., amounts, temperatures, etc.) but some experimental errors and deviations should be accounted for. Unless indicated otherwise, parts are by weight, molecular weight is weight average molecular weight, temperature is in degrees Centigrade, and pressure is at or near atmospheric.

### Example 1

### Methods

### Study Approval

All human lung experiments were approved by the Cedars-Sinai Medical Center Institutional Review Board (IRB) and were in accordance with the guidelines outlined by the IRB. Informed consent was obtained from each subject. All animal experiments were approved by the Institutional Animal Care and Use Committee at Cedars-Sinai Medical Center. All mice were housed in a pathogen-free facility at Cedars-Sinai Medical Center and had access to autoclaved water and pelleted mouse diet ad libitum.

### Human Lung Fibroblast Culture and Cell Migration and Invasion Assays

Human lung fibroblasts were isolated and cultured as previously described (Geng Y, Liu X, Liang J, Habiel D M, Kulur V, Coelho A L, Deng N, Xie T, Wang Y, Liu N et al (2019) P D-L1 on invasive fibroblasts drives fibrosis in a humanized model of idiopathic pulmonary fibrosis. JCI Insight 4; L1 Y, Jiang D, Liang J, Meltzer E B, Gray A, Miura R, Wogensen L, Yamaguchi Y, Noble P W (2011) Severe lung fibrosis requires an invasive fibroblast phenotype regulated by hyaluronan and CD44. J Exp Med 208: 1459-1471).

Briefly, human lung tissues were minced, digested, and cultured in DMEM supplemented with 15% FBS and Antibiotic-Antimycotic. Floating cells were washed out, and retained attached fibroblasts were cultured and passaged.

Fibroblast migration and invasion assays were performed as previously described (Geng et al., 2019; L1 et al., 2011). In brief, fibroblasts were loaded into the top chamber of a Corning BioCoat Control cell culture insert containing a polycarbonate membrane with 8.0 m pores (for migration) or a BioCoat Matrigel Invasion Chamber (BD Sciences; for invasion). All other settings were the same for the cell migration assay as for the cell invasion assay.

PDGF-BB (10 ng/ml, Peprotech) was used as a chemoattractant in the bottom chamber. After 24 hours culture at 37° C. with 5% CO₂, invasive cells that had passed through the matrigel layer and clung to the bottom of the insert membrane were determined as invasive fibroblasts. Cells remaining in the upper chamber were defined as non-invasive fibroblasts. For quantification of the migration and invasion indices, the membrane filters containing cells were fixed and stained with the Protocol Hema 3 stain set (Thermo Fisher Scientific) and counted in 5 randomly chosen fields per filter from triplicate filters per sample at ×40 magnification. Noninvasive cells were removed from the upper side of the filter by gentle scrubbing with a cotton swab. The cell migration or the cell invasion index was calculated as the number of cells attached to the bottom of control or Matrigel-coated membranes after 24 hours, normalized to respective control lung fibroblasts.

### Single Cell RNA-Sequencing, Data Processing and Ingenuity Pathway Analysis (IPA)

Sequencing library construction was done by the 10× genomics chromium platform as previously described (Xie et al., 2018). Cell Ranger version 1.3.1 (10× Genomics) was used to process raw sequencing data and Cell Ranger R kit version 2.0.0 and Seurat suite version 2.0.0 for downstream analysis. For clustering, principal-component analysis was performed for dimension reduction. Top 10 principal components (PCs) were selected by using a permutation-based test implemented in Seurat and passed to t-SNE for clustering visualization. Differentially expressed genes were extracted from in-1 cluster and non-in-2 cluster for IPA using the cutoff: average expression>0.1, adjusted p value (FDR)<0.05, absolute value of Log 2 fold-change>0.58.

Gene Knockdown Assay and qRT-PCT

Small interfering RNA (siRNA) knockdown assays were performed using Lipofectamin RNAiMAX Transfection Reagent following the manufacturer's protocol (Thermo Fisher Scientific). Commercial si-RNA used were listed: si-FOXF1 (Santa Cruz Biotechnology, sc-60655), si-CREBRF (Santa Cruz Biotechnology, sc-91839), si-TSC22D1 (Thermo Fisher Scientific, 16708), si-MXI1 (Santa Cruz Biotechnology, sc-35835), si-KLF9 (Santa Cruz Biotechnology, sc-37716), si-NFE2L2 (Santa Cruz Biotechnology, sc-37030), si-HMGA2 (Santa Cruz Biotechnology, sc-37994) si-DPF3 (Santa Cruz Biotechnology, sc-92150) and si-LIN00152 (Dharmacon, SMARTpool: Lincode CYTOR siRNA). Si-FENDRR were synthesized by Integrated DNA Technologies (IDT) (Table 1). RNA was isolated using RNeasy Mini kit (QIAGEN) following the manufacturer's protocol. M-NHIV Reverse Transcriptase (Progema) was used for cDNA synthesis. Gene expression was measured relative to the endogenous reference gene GAPDH using the comparative ACT method. The primer sequences used were listed (Table 1).

TABLE 1
Primer and si-RNA sequence used

| | Primer sequence | |
|---|---|---|
| Gene | Forward | Reverse |
| SEMA7A | TGTGTATTCCCTCGGTGACA | GATCTCCATCATGTTGAAGG |
| | (SEQ ID NO: 1) | (SEQ ID NO: 2) |
| F3 | TCCCCAGAGTTCACACCTTACCT | CACTTTTGTTCCCACCTGTTCA |
| | (SEQ ID NO: 3) | (SEQ ID NO: 4) |
| ITGA6 | GAGCTTTTGTGATGGGCGATT | CTCTCCACCAACTTCATAAGGC |
| | (SEQ ID NO: 5) | (SEQ ID NO: 6) |
| FENDRR | AATTGCTGGGCTGCTTTCTA | TTCACAATGGCTCAGTGCTC |
| | (SEQ ID NO: 7) | (SEQ ID NO: 8) |
| LINC00152 | TCTTCACAGCACAGTTCCTGG | GGCTGAGTCGTGATTTTCGG |
| | (SEQ ID NO: 9) | (SEQ ID NO: 10) |
| FOXF1 | GCGGCTTCCGAAGGAAATG | CAAGTGGCCGTTCATCATGC |
| | (SEQ ID NO: 11) | (SEQ ID NO: 12) |
| CREBRF | GCCATCTGAGTGGAATCGAGA | CCGTGACTTCTTAACTGCGTATT |
| | (SEQ ID NO: 13) | (SEQ ID NO: 14) |
| TSC22D1 | CTGACGACACCCCTGGTGGAT | CGATTTTGTTGTCAATAGCTAC |
| | (SEQ ID NO: 15) | CACAC (SEQ ID NO: 16) |
| MXI1 | CAACGTGCAGCGTCTGCTGGAG | CGATTCTTTTCCAGCTCATTGTG |
| | GC (SEQ ID NO: 17) | (SEQ ID NO: 18) |
| KLF9 | TGGCTGTGGGAAAGTCTATGG | CTCGTCTGAGCGGGAGAACT |
| | (SEQ ID NO: 19) | (SEQ ID NO: 20) |
| NFE2L2 | AGCCCAGCACATCCAGTCA | TGCATGCAGTCATCAAAGTAC |
| | (SEQ ID NO: 21) | AAAG (SEQ ID NO: 22) |
| HMGA2 | AAAGCAGCTCAAAAGAAAGCA | TGTTGTGGCCATTTCCTAGGT |
| | (SEQ ID NO: 23) | (SEQ ID NO: 24) |
| DPF3 | GGCTGCTGGAGATAAAACCTGA | TTCCTGGATGCTTTCCTCCTC |
| | (SEQ ID NO: 25) | (SEQ ID NO: 26) |

| si-RNA | Sequence |
|---|---|
| si-FENDRR | rCrCrArGrCrCrArUrGrUrGrArUrUrCrCrArArATT |
| #1 | (SEQ ID NO: 27) |
| si-FENDRR | rGrCrGrArUrUrGrArCrUrGrUrCrUrUrArUrArATT |
| #2 | (SEQ ID NO: 28) |

### Flow Cytometry and Fluorescence-Activated Cell Sorting (FACS)

Donor information of fresh human samples used for flow cytometry analysis is provided in Table 2. Briefly, cells were resuspended in Hank's balanced saline solution supplemented with 2% FBS, 10 mM THEPES, 0.1 mM EDTA, Antibiotic-Antimycotic. Directly conjugated antibodies used were anti-SEMA7A-PE and anti-SEMA7A-BV480 (BD Biosciences, clone KS-2), anti-anti-CD274-PE (Biolegend, clone 29E.2A3), anti-F3-PE (BD Biosciences, clone HTF-1), anti-ITGA6-PE (BD Biosciences, clone GoH3), FITC anti-human CD326 (EpCAM, Biolegend, Clone 9C4), PE/Cy7 anti-human CD31 (Biolegend, Clone WM59) and PE/Cy7 anti-human CD45 (Biolegend, Clone HI30). 4′,6-diamidino-2-phenylindole (DA-PI) was used to discriminate dead cells. Flow cytometry was performed using an LSRFortessa cell analyzer and FACS was performed on FACSAria III sorter (BD Immunocytometry Systems) and data were analyzed using FlowJo 10.2 software (Tree Star).

TABLE 2
Human sample donor information and summary of single-cell RNA-seq experiments.
(Gen = Gender, in = invasive, non-in = non-invasive)
Sample information for scRNA-seq

| | Patient information | | | Cell Number (Before QC) | | | Cell Number (After QC) | | |
|---|---|---|---|---|---|---|---|---|---|
| Sample ID | Age | Gen | Disease | in | non-in | Total | in | non-in | Total |
| CTL1 | 17 | M | normal | 4,096 | 4,450 | 8,546 | 3,966 | 4,401 | 8,367 |
| CTL2 | 21 | M | normal | 5,310 | 4,636 | 9,946 | 4,778 | 4,007 | 8,785 |
| CTL3 | 51 | F | normal | 7,701 | 2,918 | 10,619 | 6,357 | 2,340 | 8,697 |
| CTL4 | 52 | M | normal | 2,930 | 3,402 | 6,332 | 2,911 | 3,368 | 6,279 |
| IPF1 | 72 | M | IPF | 4,812 | 4,565 | 9,337 | 4,760 | 4,555 | 9,315 |
| IPF2 | 59 | M | IPF | 5,280 | 4,739 | 10,019 | 4,094 | 3,270 | 7,364 |
| IPF3 | 71 | M | IPF | 5,910 | 5,175 | 11,085 | 4,586 | 3,238 | 7,824 |
| IPF4 | 73 | M | IPF | 3,093 | 3,801 | 6,894 | 3,067 | 3,780 | 6,847 |

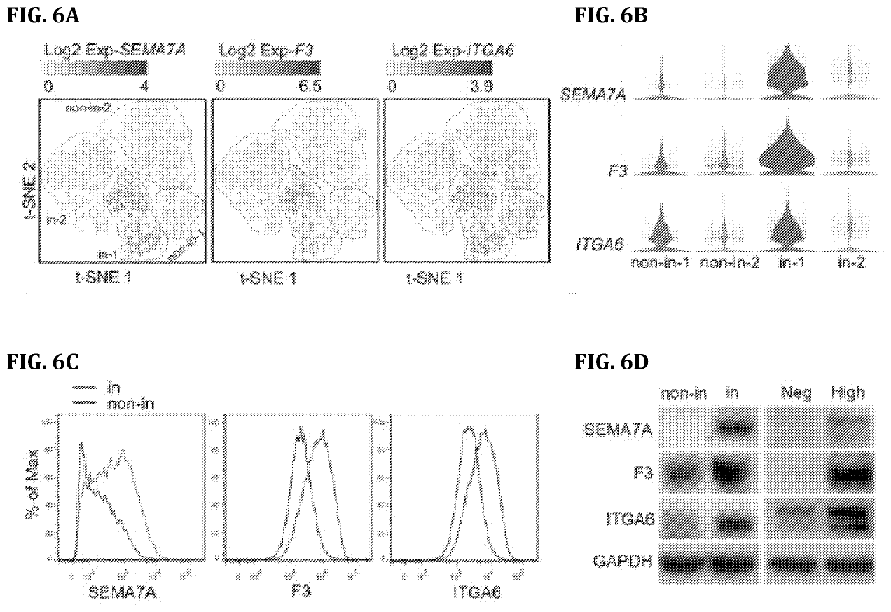

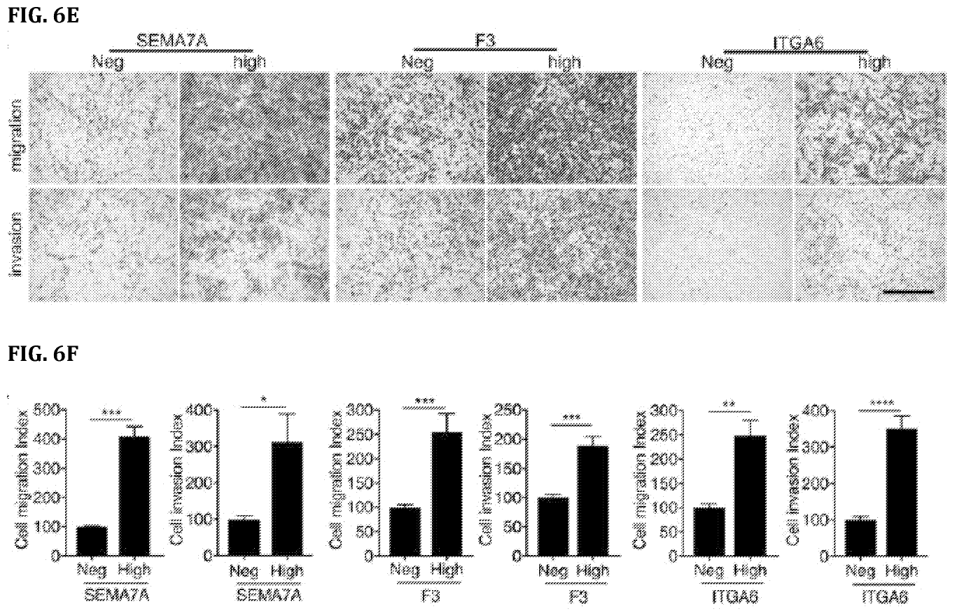

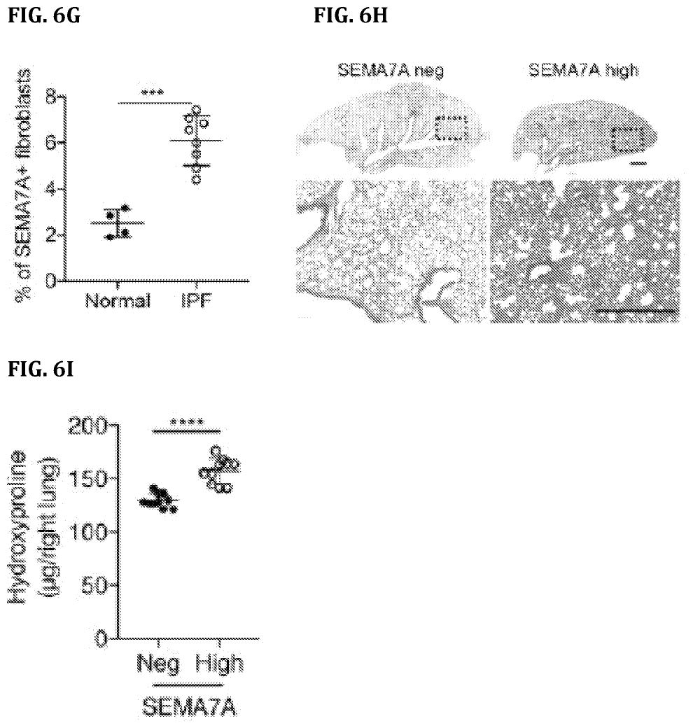

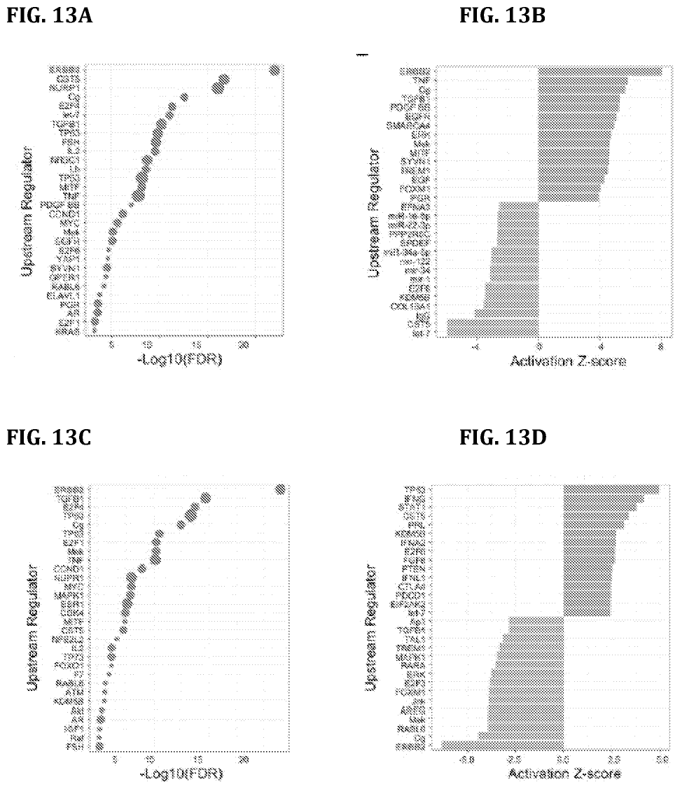

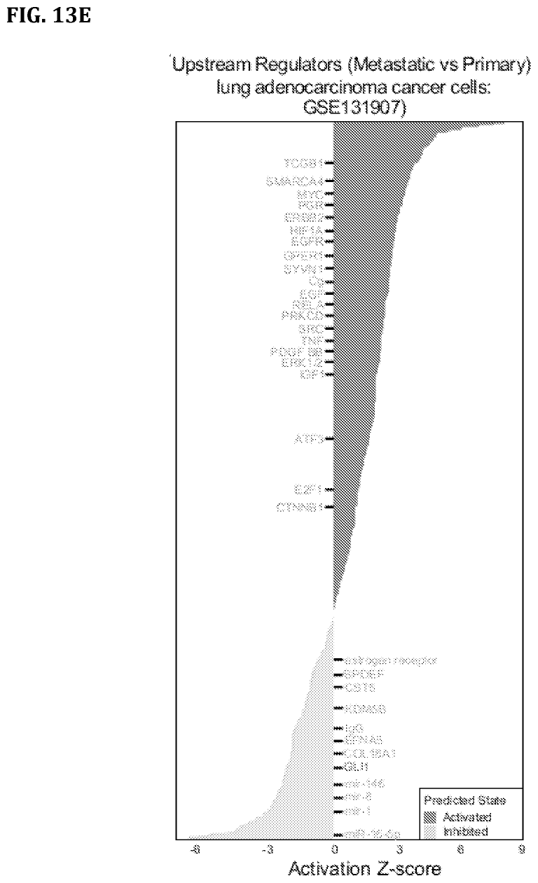

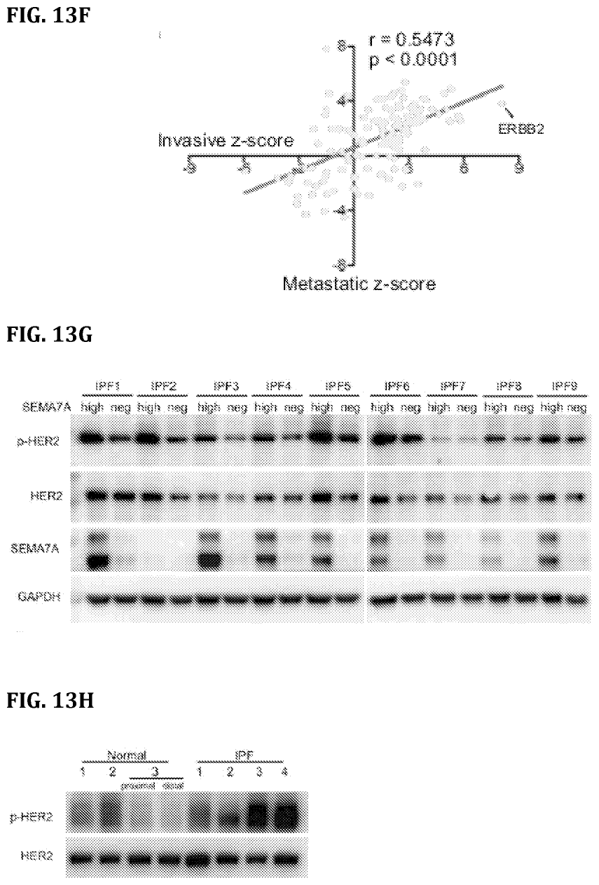

Sample information for FIG. 6G and FIG. 13H

| FIG. 6G | Age | Gen | Disease | Part | FIG. 13H | Age | Gen | Disease | Part |
|---|---|---|---|---|---|---|---|---|---|
| sample 1 | na | na | normal | na | sample 1 | 78 | M | normal | na |
| sample 2 | 2 | M | normal | na | sample 2 | 18 | M | normal | na |
| sample 3 | 78 | M | normal | na | sample 3 | 78 | F | normal | D + P |
| sample 4 | 18 | M | normal | na | sample 4 | 55 | F | IPF | na |
| sample 5 | 71 | M | IPF | D + P | sample 5 | na | na | IPF | na |
| sample 6 | na | na | IPF | D + P | sample 6 | 58 | M | IPF | na |
| sample 7 | 65 | M | IPF | na | sample 7 | 71 | M | IPF | na |

| | | | | | |
|---|---|---|---|---|---|
| sample 8 | 58 | M | IPF | D + P | na, not available |
| sample 9 | 55 | F | IPF | na | D + P, distal and proximal |

### Western Blot

Western blot analysis was performed as previously described (Geng et al., 2019). Donor information for IPF fibroblasts (Geng et al., 2019) and fresh human samples (Table 2) were provided. Antibodies were used: anti-GAPDH (Cell signaling technology, clone 14C10), anti-SEMA7A (Novus Biologicals, NBP1-86555), anti-F3 (Cell signaling technology, clone E9M6T), anti-ITGA6 (Cell signaling technology, 3750S), anti-FOXF1 (R&D system, AF4798), anti-CREBRF (abcam, ab26262), anti-TSC22D1 (Novus Biologicals, NBP2-46238), anti-MXI1 (ABclonal, A12098), anti-KLF9 (Santa Cruz Biotechnology, clone A-5) and anti-NFE2L2 (Cell signaling technology, clone D1Z9C).

### RNAscope Assay In Situ Hybridization Assay

In situ hybridization for FENDRR and FOXF1 RNA expression analysis was performed using RNAscope Chromogenic Detection Kit following the manufacturer's protocol (Advanced Cell Diagnostics, ACD). Cells were harvested by trypsin digestion and attached to glass slides by Cytospin 4 Cytocentrifuge (Thermo Fisher Scientific). Then the cells were fixed by neutral buffered formalin and dehydrated by a gradient of ethanol. After rehydration, the cells were treated with hydrogen peroxide and Protease III successively and incubated with probe, Amp 1-FL, Amp 2-FL, Amp 3-FL, Amp 4-FL in a humidity slide rack at 40° C. in oven. The processed slides were mounted in Vectashield (Vector Labs) containing DAPI and photographed with a confocal microscope (Carl Zeiss Microscopy). For the score of the RNAscope, at least 100 cells were measured in each image and at lease least 3 images were measured for each group. For the score system of the RNAscope assay, no staining or <1 dot/10 cells gives a score of 0, 1-3 dots/cell gives a score of 1, 4-9 dots/cell and none or very few dot clusters gives a score of 2, 10-15 dots/cell and/or <10% dots are in clusters gives a score of 3 and >15 dots/cell and/or >10% dots are in clusters gives a score of 4 according to the manufacturer's instructions.

### Cell Growth Rate and Cell Viability Assay

Cell growth rate of si-CTL and si-FENDRR fibroblasts were measured by IncuCyte ZOOM Live Cell Analysis System (Essen BioScience). The viability of lapatinib-treated fibroblasts or control fibroblasts was examined by Calcein AM Cell Viability Assay Kit (Trevigen).

### Humanized SCID Mouse Model of IPF

Humanized SCID mouse model of IPF was generated as previously reported (Trujillo et al., 2010). Female NOD-scid-IL2Rγc−/− (NSG) mice (6 to 8 weeks old) were purchased from the Jackson Laboratory. The NSG mice received single-cell preparations of SEMA7A high and negative IPF lung fibroblasts (0.5×10⁶ cells/mouse) via tail vein injection. For ERBB2 inhibitor studies, mice were treated with 0.5% CMS-Na (control group) or 50 mg/kg Lapatinib (Tocris Bioscience) every other day from day 35 to day 63. For the anti-ERBB2 antibody studies, mice were injected with IgG1 (InVivoPlus human IgG1 isotype control, Bio X Cell) or anti-HER2 (Pertuzumab, kindly provided by Genentech) twice per week, 100 μg/mouse from day 35 to 63. Lung fibrosis was assessed on day 63 after fibroblast transfer. The left lobe was used for histology and right lobes were used for hydroxyproline assay after right ventricle perfusion to clear the blood in lungs.

### Bleomycin Instillation and Lapatinib Treatment

Under anesthesia, mouse trachea was surgically exposed and 1.25 U/kg bleomycin (Hospira, Lake Forest, IL) in PBS was instilled into the mouse trachea with a 25-G needle inserted between the cartilaginous rings of the trachea. Control animals received saline alone. The tracheostomy site was sutured, and the animals were allowed to recover. Lapatinib or vehicle control was gavaged every day. Mice were euthanized 21 days after bleomycin treatment, and lung tissues were collected for experiments.

### Statistics

Data are expressed as the mean±SEM. All experiments were repeated 2 or more times. Student's 2-tailed t test was used for comparing differences between 2 groups. One-way or 2-way ANOVA followed by Tukey-Kramer test was used for multiple comparisons. Significance was set at P<0.05. GraphPad Prism software 8.0 was used for statistical analysis. Wilcoxon tests were conducted to compare gene transcript levels in single cell RNA-seq.

Data and materials availability: All data associated with this study are presented herein. Single cell RNA-seq and total RNA-seq data have been uploaded to GEO (GSE137025 and GSE137026). Total RNA-seq on invasive and non-invasive IPF fibroblasts were published previously (accession number, GSE118933).

### Results

### Clustering of Invasive and Non-Invasive Lung Fibroblasts by Single Cell RNA-Seq.

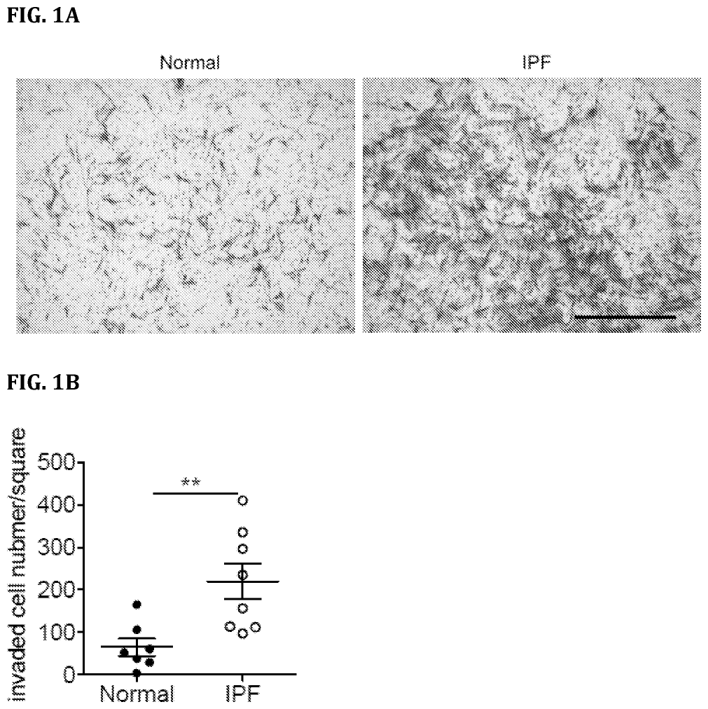

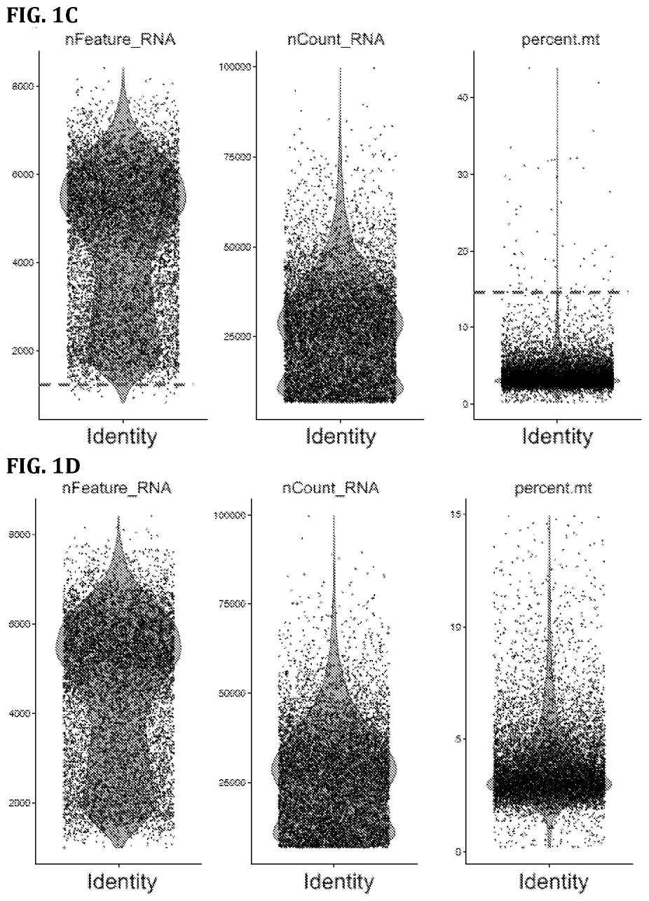

Severe lung fibrosis required an invasive fibroblast phenotype and IPF lung fibroblasts showed a significant increased capacity in invasiveness compared to normal lungs (FIGS. 1A and 1i). To better demonstrate the underlying mechanisms, we investigated single cells isolated from invasive and non-invasive lung fibroblasts from four IPF patients and four healthy controls (CTL) using GemCode™ system (10× Genomics), which is based on a high-throughput Droplet-based platform as we have previously reported with mouse lung fibroblasts. We profiled 72,818 individual cells, removed 9,340 low-quality cells (Materials and Methods, and FIGS. 1C and 1D) and retained 34,519 invasive and 28,959 non-invasive lung fibroblasts for subsequent analyses (Table 2). Table 2 provides the human sample donor information and the number of individual invasive and non-invasive lung fibroblasts profiled in each IPF or CTL sample before quality control (QC) and after QC.

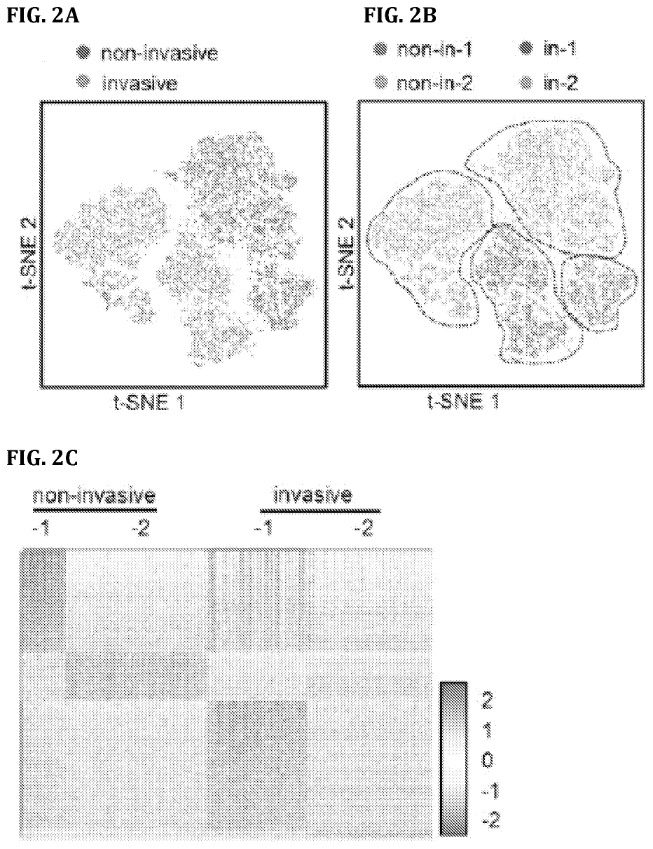

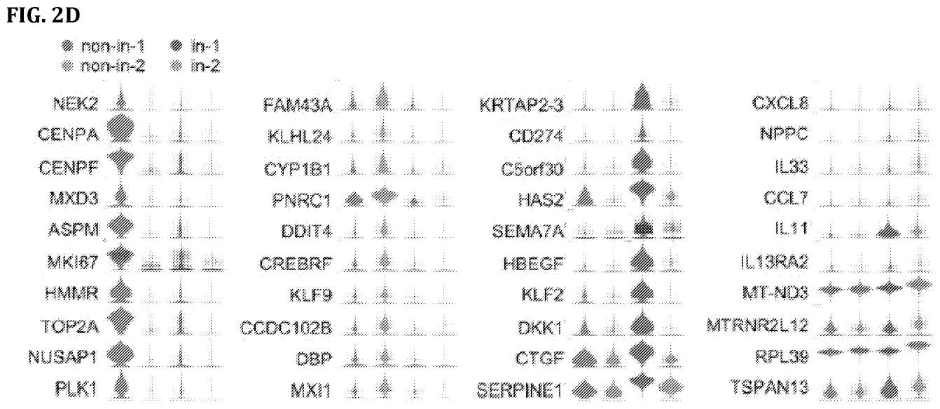

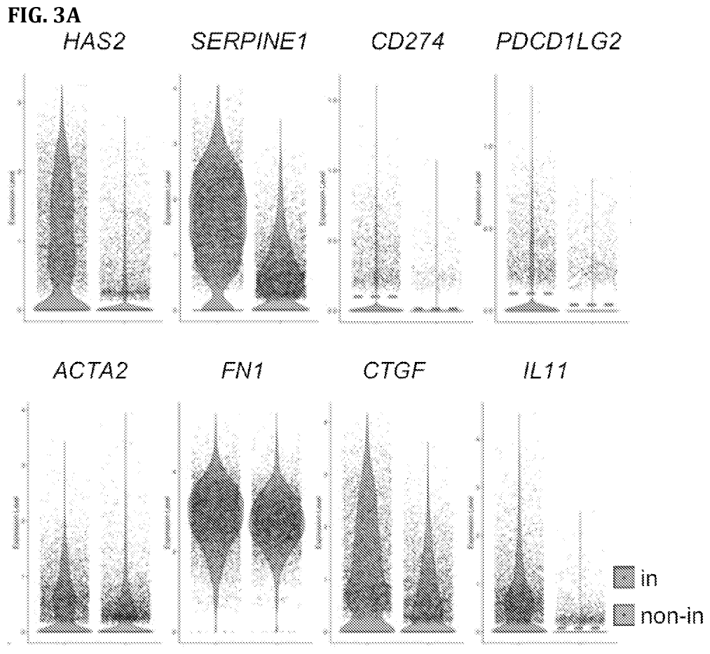

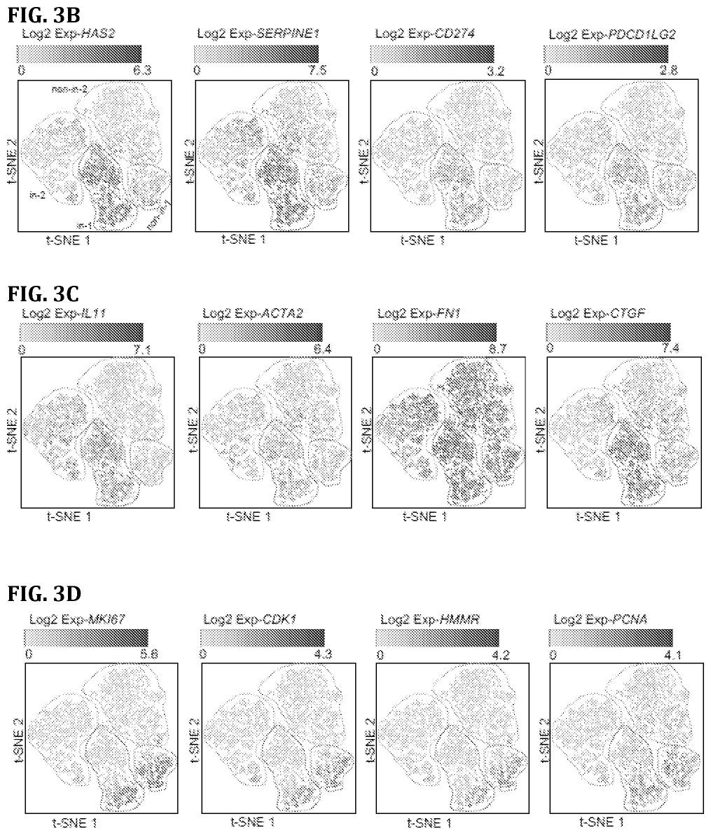

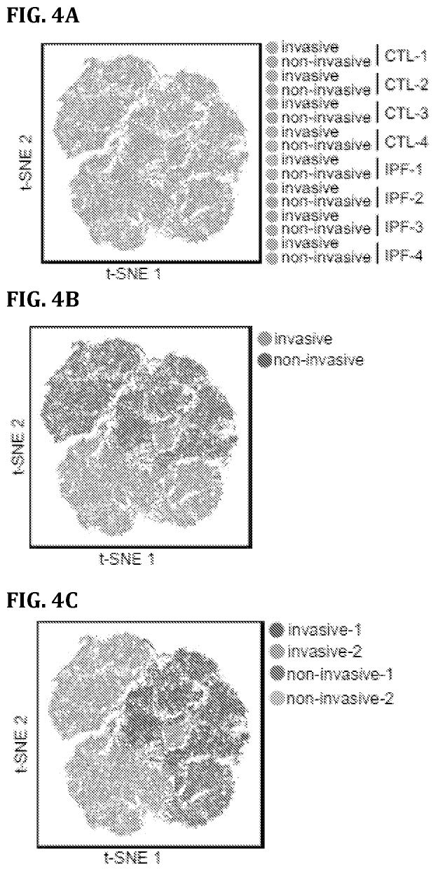

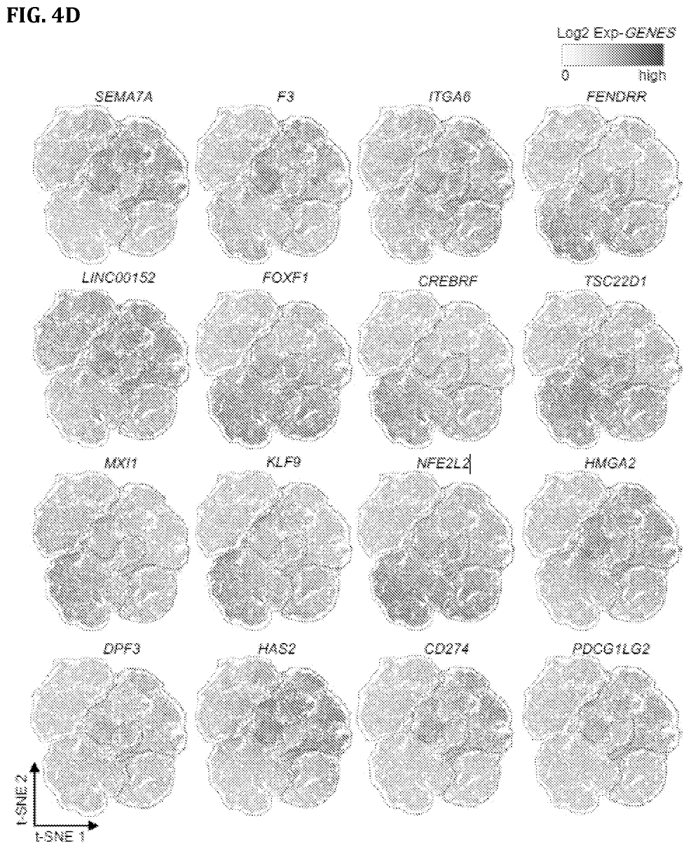

We then grouped the cells by nonlinear dimensionality reduction (t-distributed stochastic neighbor embedding (t-SNE)). There were two distinct cell clusters in both invasive and non-invasive lung fibroblasts (FIG. 1A) and we defined them as invasive-1 (in-1), invasive-2 (in-2), non-invasive-1 (non-in-1) and non-invasive-2 (non-in-2) (FIG. 1). We explored the four clusters and profiled them by significantly expressed genes (FIG. 1C). Signature genes expression across four clusters were confirmed by violin plot analysis of the single cell RNA-seq (FIG. 1D) and more gene expression signatures for each cell clusters were defined (Table 3). HAS2, SERPINE1 (PAI-1), CD274 (PD-L1), PDCDILG2 (PD-L2) and IL11 are reported to promote fibrogenesis as markers of invasive fibroblasts in fibrotic tissue and showed extremely high expression in invasive cell clusters (FIGS. 3A and 3B), which validated our single cell RNA-seq data. Several activated fibroblast (myofibroblast)-specific genes were also determined (FIGS. 3A and 3C). We identified non-invasive-1 and part of invasive-1 cell clusters as proliferation cells using cell proliferation markers MKI67, CDK1, HMMR and PCNA (FIG. 2D and FIG. 3D). We then combined all of the cells of the normal (N=4) and IPF samples (N=4) and identified the same clustering pattern (FIG. 4A-4C). As each cluster of the four clusters of IPF-1 fibroblasts was associated with a distinct cell state, these profiles demonstrated excellent reproducibility in the lung fibroblasts from the other IPF patients (FIGS. 4A-4C). Strikingly, there were similar patterns in lung fibroblasts from healthy controls (FIGS. 4A-4C) and proliferative markers, and fibrotic markers, HAS2, CD274 and PDCDILG2 (FIG. 4D), showed similar patterns among all four samples.

TABLE 3
Cell-cluster specific signature genes

Anti-sense
GPCR
Growth factor
Ion channel
Kinase
Ligand-depend nuclear receptor
LincRNA
Peptidase
Phosphatase
Transcription factor
Translation regulator
Transporter
Significance cut-offs: average expression >0.1, adjusted p value (max) <0.05, Log2 fold-change >0.58

### Differentiation Potential of the Four Clusters.

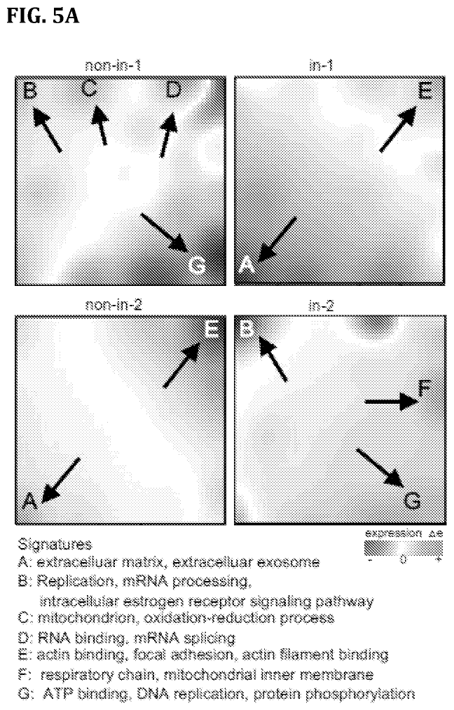

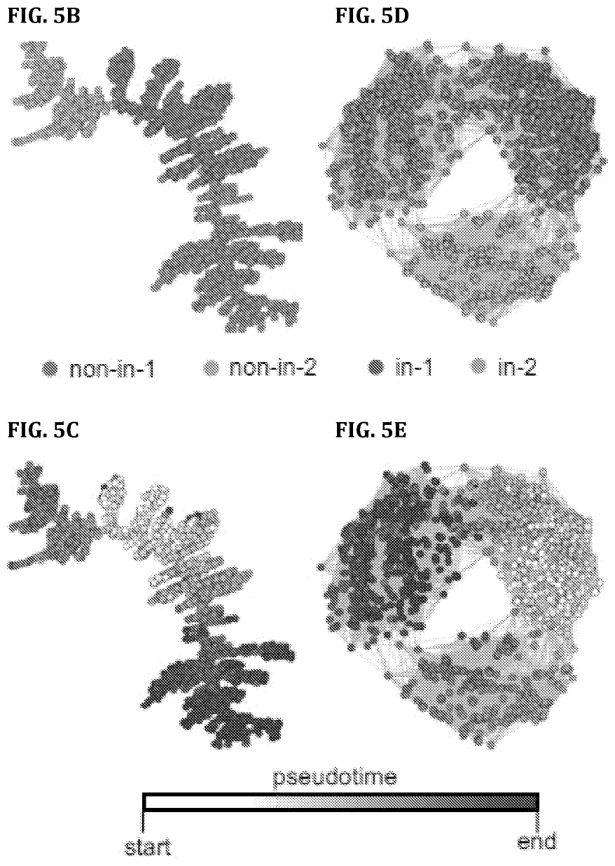

A customizable suite of single-cell R-analysis tools (SCRAT) has been developed to explore single cell transcriptome heterogeneity based on self-organizing maps (SOM) machine learning. The analysis by SCRAT revealed that the four clusters showed variable signatures and each cluster exhibited specific gene signatures, including extracellular matrix, extracellular exosome, replication, mRNA processing, intracellular estrogen receptor signaling pathway, mitochondrion, oxidation-reduction process, RNA binding, mRNA splicing, actin binding, focal adhesion, actin filament binding, respiratory chain, mitochondrial inner membrane, ATP binding, DNA replication and protein phosphorylation (FIG. 5A). Notably, the invasive-1 cell cluster showed high focal adhesion activity, which was consistent with our previous report (Geng et al., 2019).

We then projected the clusters onto the SCRAT for cell similarity and pseudotime analysis, which provides information inferring lineage trajectories from single-cell expression data in the form of 2D bifurcation. We found that the correlation-spanning tree and trajectory report displayed a directed hierarchical relation of the clusters, starting from non-invasive-2 cluster and bifurcating to other clusters. Interestingly, non-invasive-2 bifurcated to non-invasive-1 and invasive-2, whereas non-invasive-1 diverged to invasive-1 sequentially (FIGS. 5B-5E).

### Cell Surface Markers Distinguish Invasive and Non-Invasive Fibroblasts.

We reported that immune checkpoint ligand CD274 (also known as PD-L1) was significantly upregulated on invasive lung fibroblasts and was required for the invasive phenotype of IPF fibroblasts. CD274 drove lung fibrosis in a humanized IPF model in mice (Geng et al., 2019). Here the single cell RNA-seq revealed that both CD274 and PDCD1LG2 (also known as PD-L2) were upregulated in the invasive fibroblasts, which validated our previous data at RNA level (FIG. 2D, and FIGS. 3A and 4D).

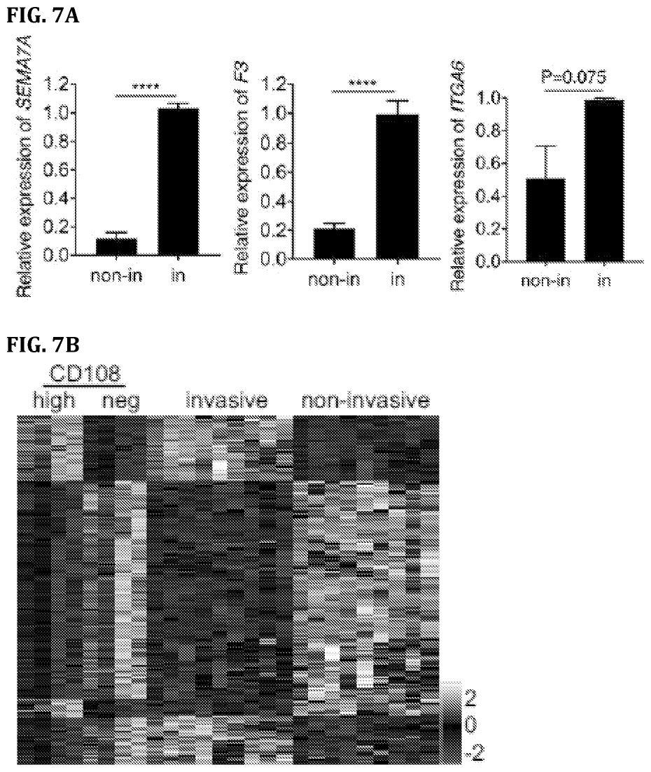

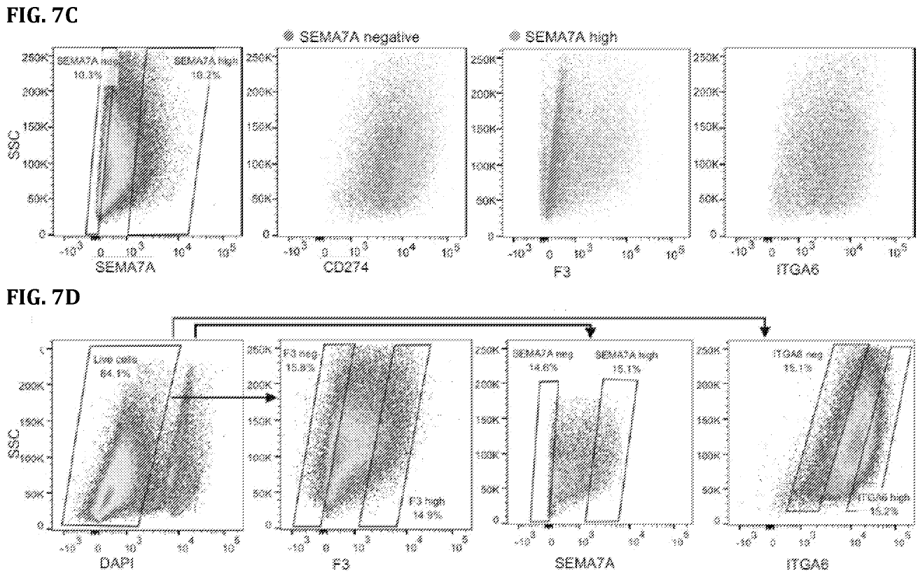

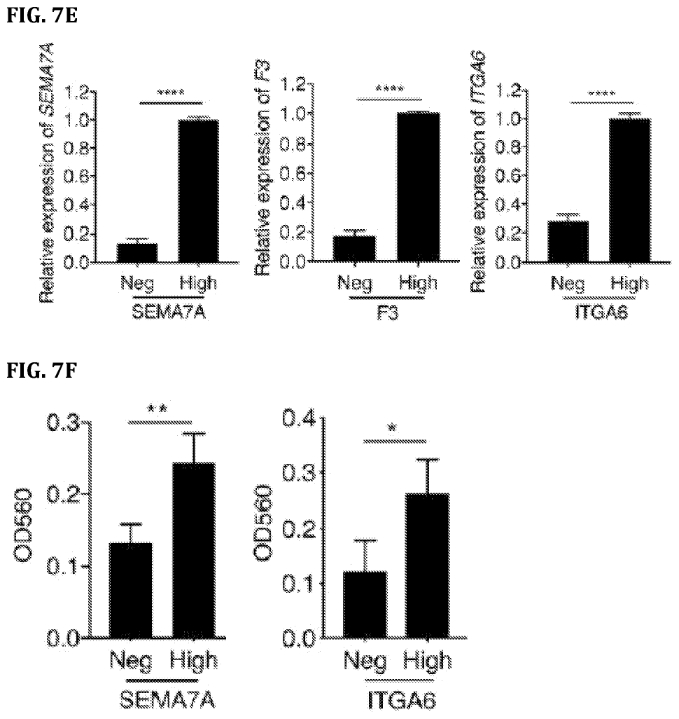

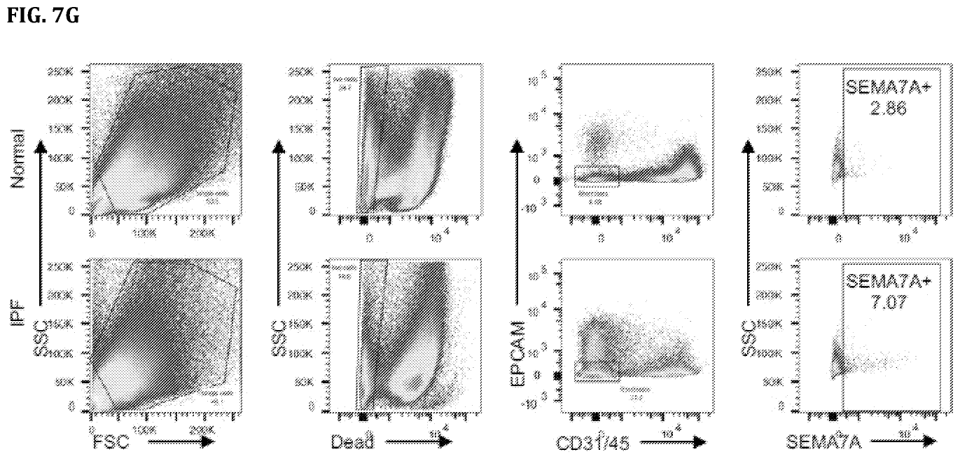

To better distinguish invasive and non-invasive lung fibroblasts, we screened six potential cell surface markers by RNA, Western, flow cytometry, as well as invasiveness. Several of them were not consistent between RNA, protein expression and cell surface expression. By RNA-seq data analysis we successfully found that SEMA7A (CD108), F3 (CD142) and ITGA6 (CD49f) were significantly upregulated in the invasive fibroblasts cluster (FIGS. 6A-6B and 4D). Cell surface, total protein and RNA expression of SEMA7A, F3 and ITGA6 were respectively validated by flow cytometry (FIG. 6C), Western blot (FIG. 6D) and qRT-PCR (FIG. 7A), which suggested that SEMA7A, F3 and ITGA6 could be the cell surface markers of invasive lung fibroblasts. To confirm this, we performed RNA-seq on flow sorted SEMA7A^{high} and SEMA7A^{negative} fibroblasts from IPF lungs and compared the RNA-seq data with invasive and non-invasive fibroblast data we recently published (Geng et al., 2019). We found that SEMA7A^{high} and invasive fibroblasts showed similar transcriptome profiles, while SEMA7^{negative} and non-invasive fibroblasts showed similar transcriptome profiles, respectively (FIG. 7B). Furthermore, flow cytometry revealed that cell surface expression of CD274, F3, and ITGA6 showed significant correlation with SEMA7A expression (FIG. 7B).

To further demonstrate the functional roles of SEMA7A, F3, and ITGA6 in fibroblast invasion, we flow sorted fibroblasts based on their cell surface expression (FIGS. 6C, 7D, and 7E). We found that cell migration and invasion were significantly increased in the cell surface protein high fibroblasts (FIGS. 6E and 6F), which suggested that these proteins could be used to identify invasive lung fibroblasts. Moreover, both SEMA7A^{high} and ITGA6^{high} fibroblasts showed greater adherent capacity than SEMA7A^{negative} and ITGA6^{negative} fibroblasts, respectively (FIG. 7F), consistent with previous reports (Geng et al., 2019).

Importantly, IPF lungs showed higher percentages of SEMA7A positive fibroblasts compared to normal lungs by flow cytometry on freshly isolated lung fibroblasts (FIGS. 6G and 7G). We have previously generated a humanized SCID IPF model, in which the immunodeficient mice NOD-scid-IL2Rγc^{-/-} (NSG) received human lung fibroblasts. Although lung fibrosis was not as acute or severe as belinomycin-induced pulmonary fibrosis, lung collagen deposition and increased expression of matrix genes were clearly identified in this model. As predicted, SEMA7A^{high} fibroblasts developed significantly more lung fibrosis than the mice receiving SEMA7^{negative} fibroblasts in a humanized severe combined immunodeficient (SCID) IPF model in vivo, although SEMA7^{negative} fibroblasts also caused lung fibrosis to a certain degree (FIG. 6H). Hydroxyproline quantification showed a 33.70% increase with SEMA7A^{high} fibroblast injection compared to that of SEMA7A^{negative} fibroblasts (FIG. 6I). These data show that these cell surface markers of invasive fibroblasts are not just “markers”, but are also functional in mediating fibroblast behaviors.

### LncRNA FENDRR was Down Regulated in Invasive Fibroblasts.

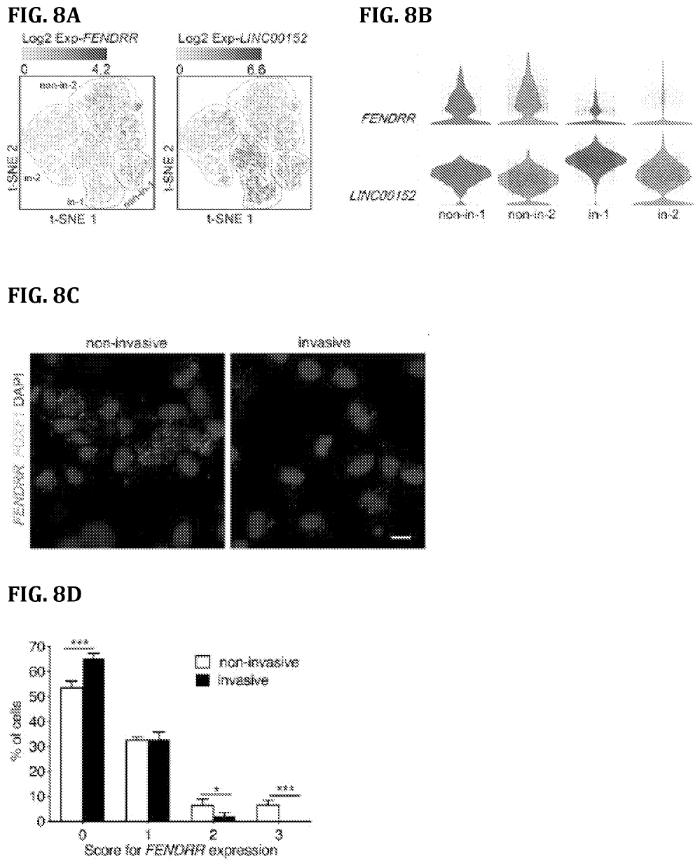

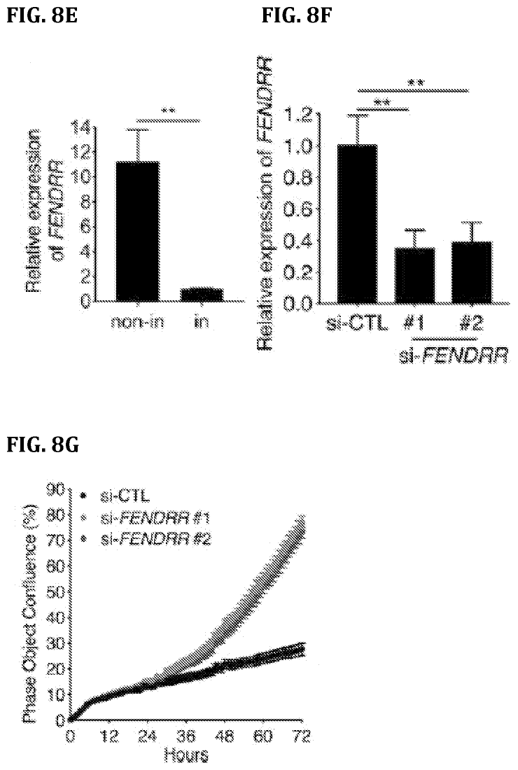

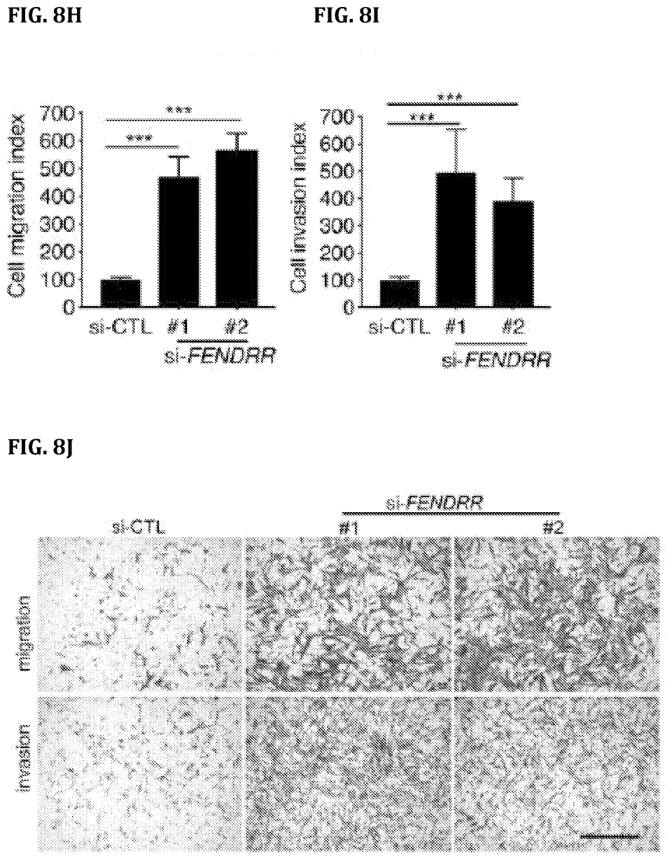

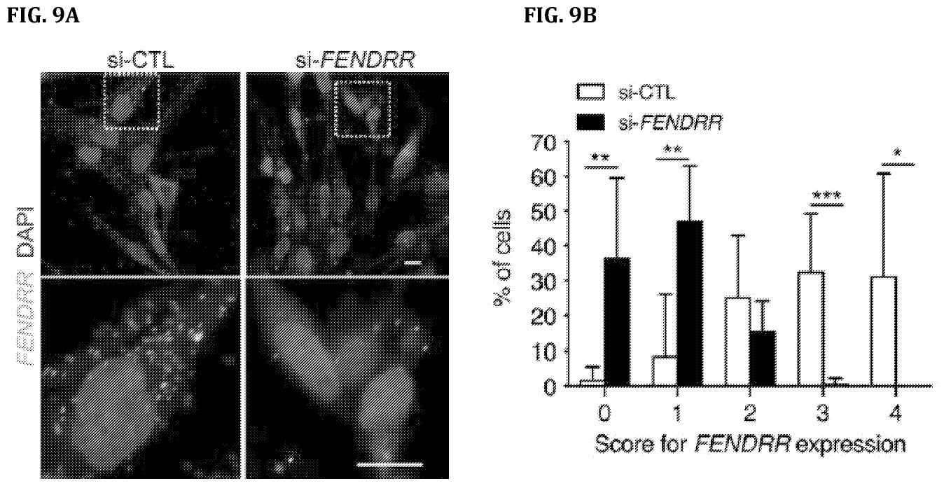

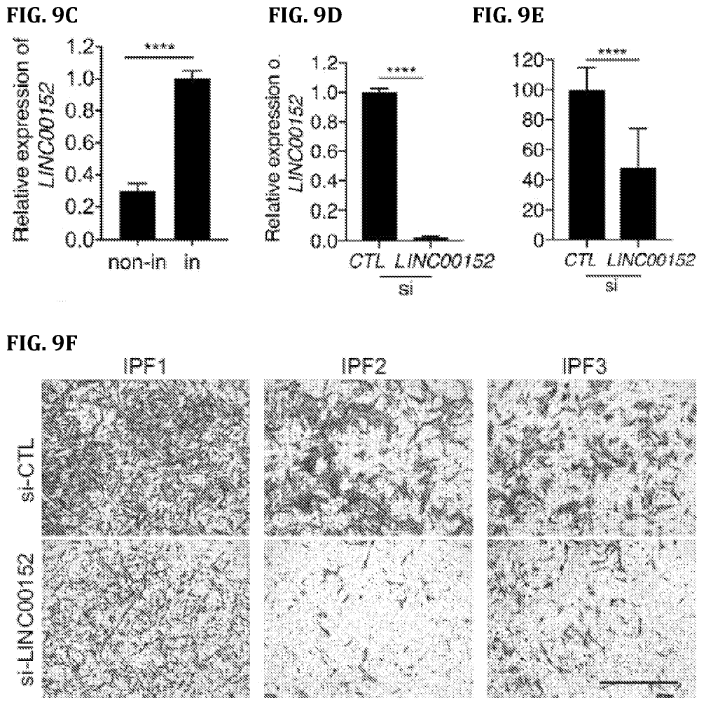

Recent studies have revealed that lncRNAs are critically involved in fibrotic diseases such as IPF. To uncover the role of lncRNAs in fibroblast invasion, two lincRNAs, FENDRR and LINC00152, were further investigated. FENDRR, FOXF1 adjacent non-coding RNA, was significantly down regulated in both invasive clusters (FIGS. 8A, 8B, and 4D). We validated the single cell RNA-seq data of FENDRR expression by RNAscope (FIGS. 8C and 8D) and qRT-PCR (FIG. 8E). To determine if the downregulation of FENDRR in lung fibroblasts would impact cell migration and invasion, we performed small interfering RNA (siRNA) knockdown assays (FIGS. 8E, 9A, and 9B). Knockdown of FENDRR in lung fibroblasts from IPF patients significantly promoted fibroblast growth (FIG. 8G), migration and invasion (FIGS. 8H-8J). LINC00152 showed an extremely high expression level in invasive fibroblasts compared to non-invasive fibroblasts (FIGS. 8A, 8B, and 4D), which was confirmed by qRT-PCR (FIG. 9C). Knockdown of LINC00152 in SEMA7A^{high} invasive fibroblasts significantly blunted fibroblast invasion (FIGS. 9D-9F).

### Identification of Transcription Factors to Distinguish Invasive and Non-Invasive Fibroblasts.

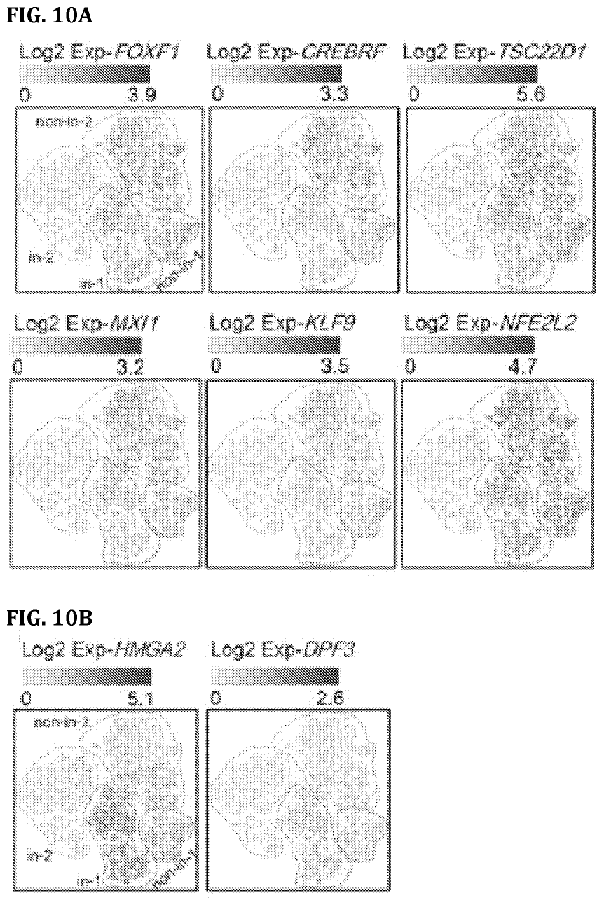

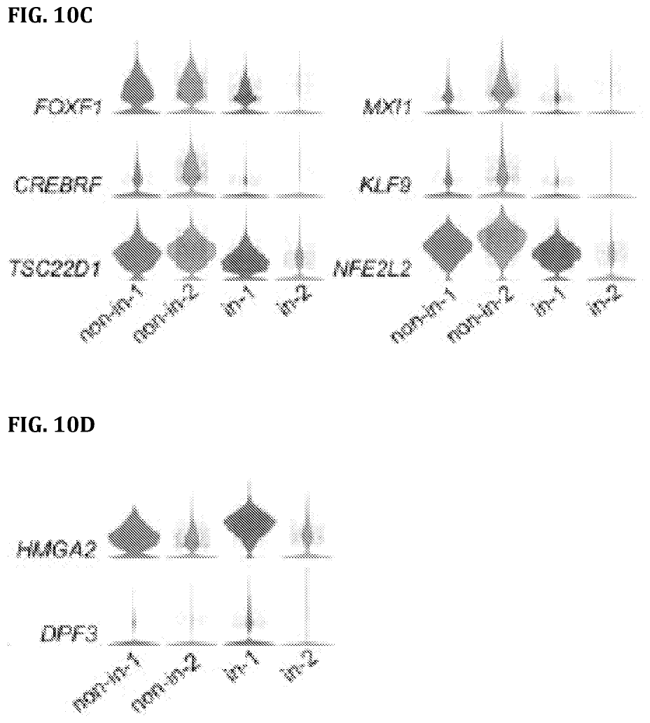

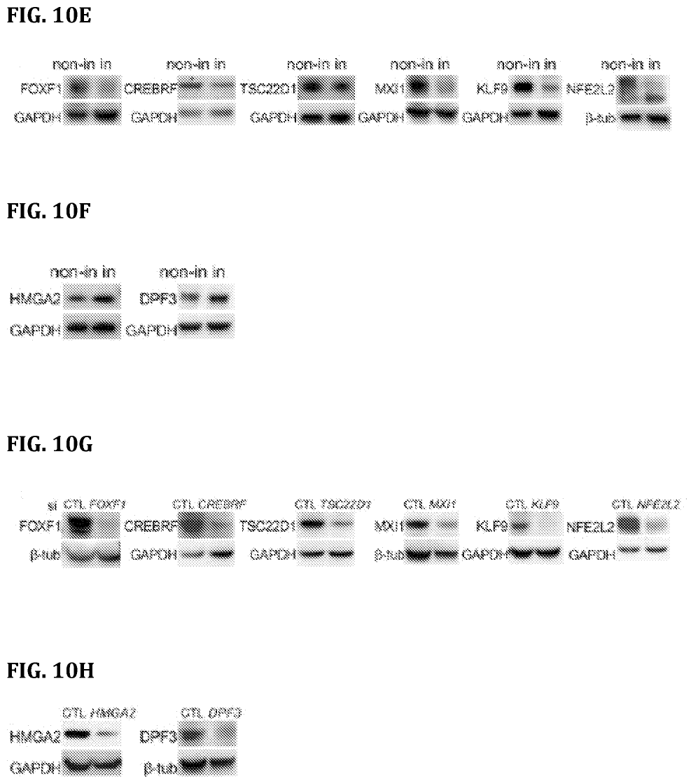

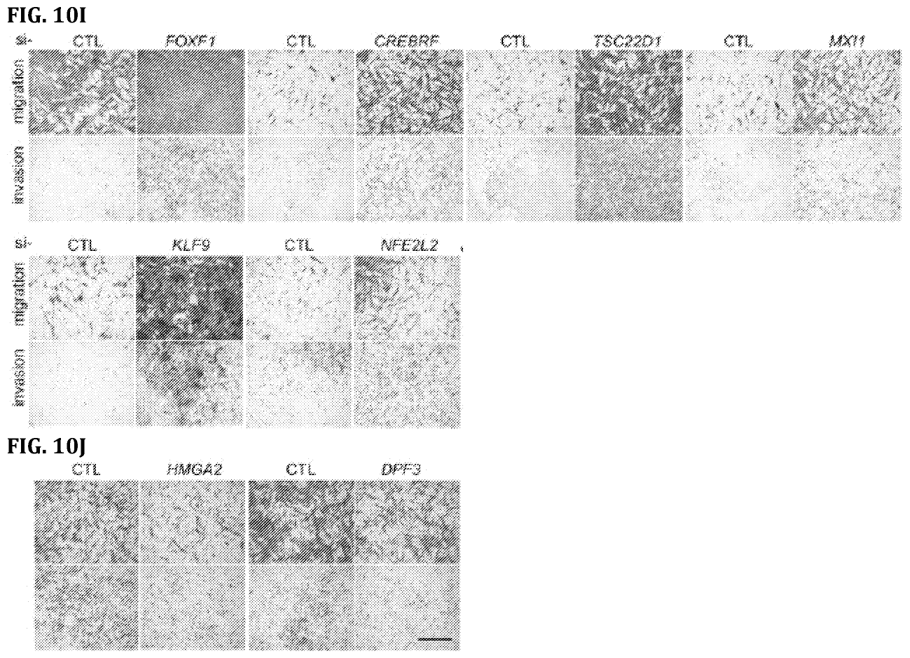

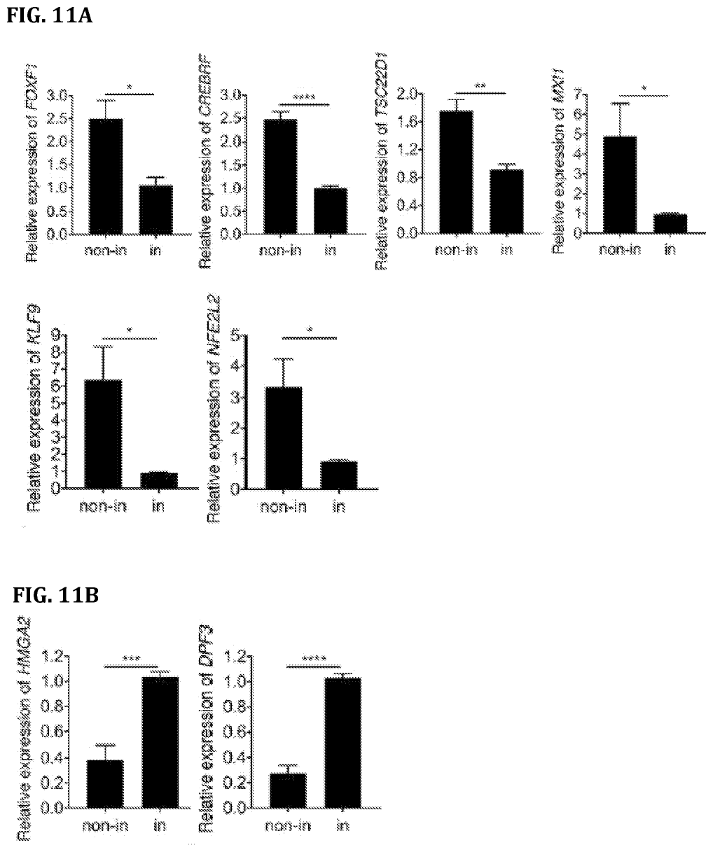

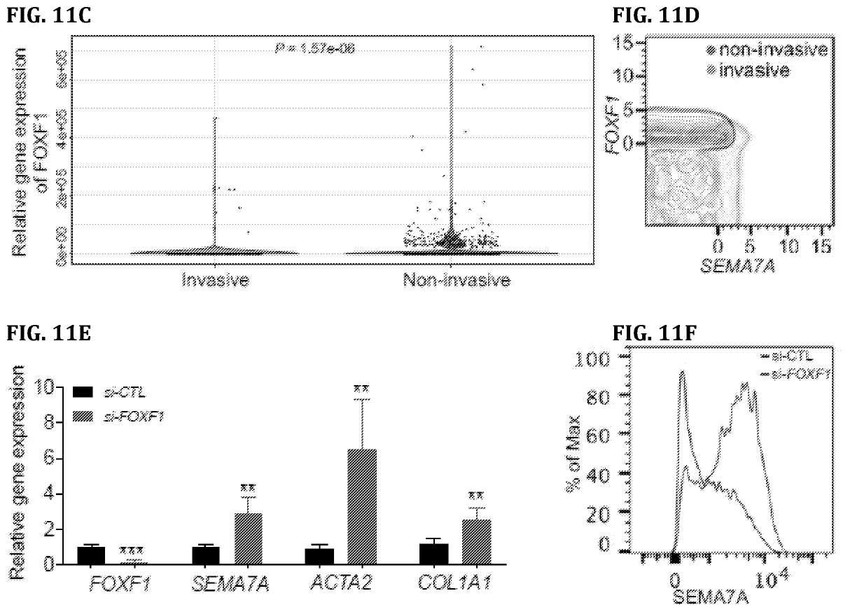

Next, we identified the critical transcription factors, which showed specific expression patterns and might regulate fibroblast invasion. By single cell RNA-seq analysis, we listed differentially expressed transcription factors (Table 2). Among 26 transcription factors screened, we found that mRNAs for FOXF1, CREBRF, TSC22D1, MXI1, KLF9 and NFE2L2 were significantly downregulated in invasive fibroblast clusters (FIGS. 10A, 10C, and 4D), while mRNAs for HMGA2 and DPF3 were upregulated in cluster invasive-1 (FIGS. 10B, 10D, and 4D). We validated the single cell RNA-seq data by Western blot (FIGS. 10E and 10F) and qRT-PCR (FIGS. 11A and 11B).

Foxf1 was recently reported to inhibit myofibroblast invasion, collagen secretion and pulmonary fibrosis in a bleomycin-induced fibrosis mouse model. Here in our single cell RNA-seq data, FOXF1 was significantly downregulated in invasive clusters, which was further confirmed by single cell Western blot (FIG. 11C), and showed negative correlation with the expression of SEMA7A, which is an invasive fibroblast surface marker (FIG. 11D). Knockdown of FOXF1 in lung fibroblasts from IPF patients significantly increased the expression of fibrosis associated markers, ACTA2, COL1A1 and FN1 (FIG. 11E). In si-FOXF1 fibroblasts, there was a significant increase in both RNA (FIG. 11E) and cell surface protein level (FIG. 11F) of SEMA7A.

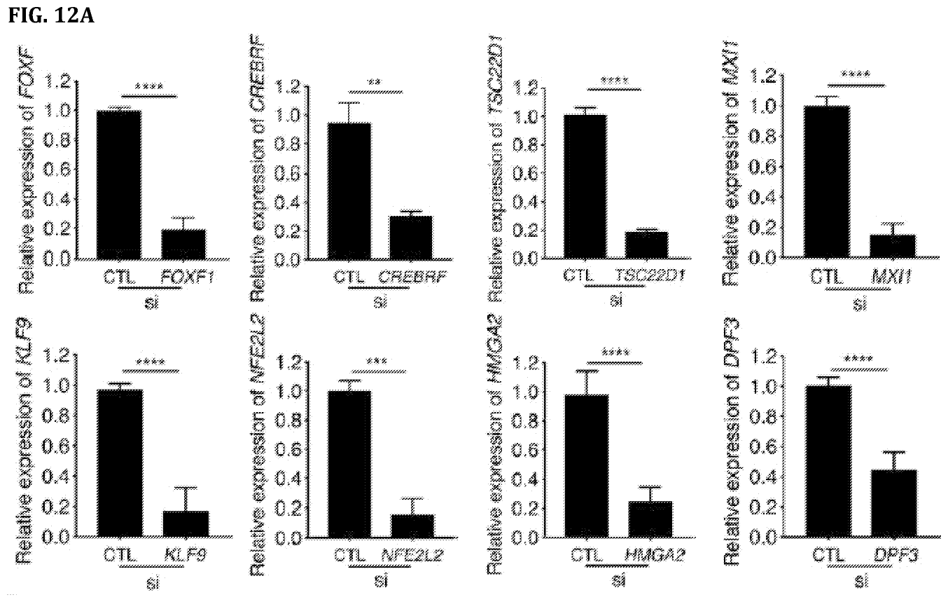

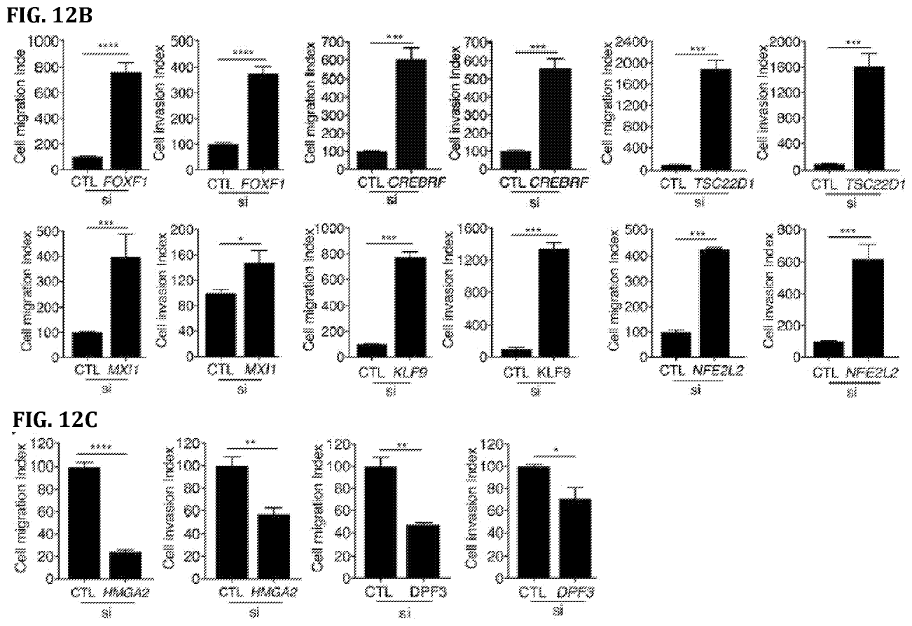

To confirm that these transcription factors were necessary for the invasiveness of lung fibroblasts, we performed siRNA knockdown assays for all the transcription factors identified. Western blot (FIGS. 10G and 10H) and qRT-PCR (FIG. 12A) were performed to evaluate the efficiency of the knockdown assays. Knockdown of FOXF1, CREBRF, TSC22D1, MXI1, KLF9 and NFE2L2 significantly promoted fibroblast migration and invasion (FIGS. 10I and 12B) and knockdown of HMGA2 and DPF3 blunted fibroblast migration and invasion (FIGS. 10J and 12C). These data show that these transcription factors drive a genetic program in promoting fibroblast invasion.

### Activated ERBB2 (HER2) Signaling Pathway in Invasive Lung Fibroblasts.

As most of the invasive signature genes were from invasive-1 cluster and most of the non-invasive signature genes were from non-invasive-2 cluster (Table 2), the specific genes of these two clusters were imported into Ingenuity Pathway Analysis (IPA), a tool to investigate possible interactions of differently regulated signaling pathway. Interestingly, the ERBB2 (HER2) was the most activated upstream regulator in the invasive-1 cluster indicated by the p-value of overlap (FIG. 13A) and activation z-score (FIG. 13B). In contrast, ERBB2 was the most inhibited upstream regulator in non-invasive-2 cluster indicated by the p-value of overlap (FIG. 13C) and activation z-score (FIG. 13D). These data demonstrated the ERBB2 signaling pathway as the key regulator to drive lung fibroblast invasion.

Many of the top upstream regulators of invasive fibroblasts, including ERBB2, EGFR, TNF, TP63, TP53 and SMARCA4, were actively involved in tumor metastasis (FIGS. 13A-D). The regulatory network by IPA analysis combining the upstream regulators and the canonical pathways revealed tumor cell invasion signaling as the core activated pathway of the network. Based on these observations, the regulatory programs of the invasive fibroblasts are closely associated with the signalings regulating tumor metastasis. To confirm that, a recently published scRNA-seq dataset on normal lung tissue and primary and metastatic lung adenocarcinoma was re-accessed and the primary and metastatic cancer cells and their specific gene expression programs were identified. IPA analysis revealed the upstream regulators of the metastatic cancer cells based on the specific gene expression (FIG. 13E). Surprisingly, among the top activated upstream regulators of invasive fibroblasts, most were significantly activated in metastatic cancer cells (20/30), while many of the inhibited upstream regulators of invasive fibroblasts were also appreciably inhibited in metastatic cancer cells (11/30) (FIG. 13E). Analysis of all the shared upstream regulators showed significant positive correlations between invasive fibroblasts and metastatic cancer cells (FIG. 13F). These data show that invasive lung fibroblasts shared similar genetic regulatory programs with metastatic lung cancer cells which were possibly determined by HER2 signaling.

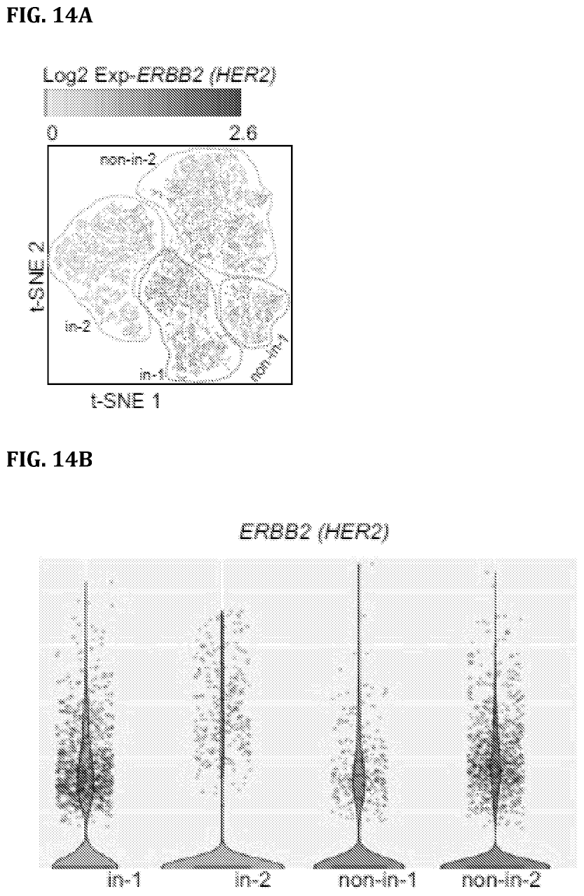

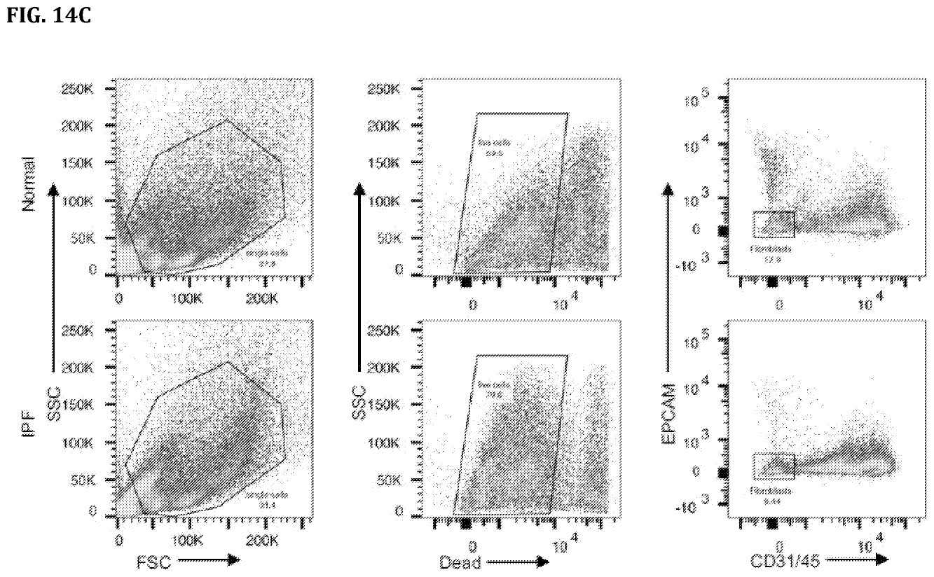

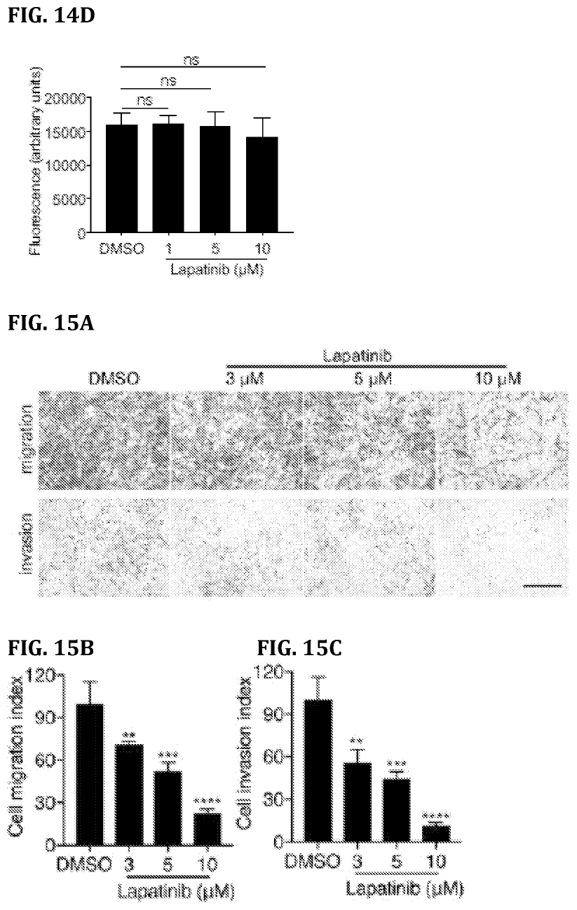

We then checked ERBB2 expression in invasive and non-invasive fibroblasts in IPF lungs and we found similar expression levels of ERBB2 in invasive and non-invasive clusters (FIGS. 14A and 14B). To confirm the activation of the HER2 signaling in invasive fibroblasts, we collected SEMA7A high and negative fibroblasts as invasive and non-invasive fibroblasts by FACS from fibroblasts of nine IPF patients. Western blot analysis on the SEMA7A high and negative fibroblasts revealed that SEMA7A^{high} fibroblasts showed significant high phospho-HER2 (p-HER2) expression compared to SEMA7A negative fibroblasts (FIG. 13G). Importantly, IPF lung fibroblasts showed higher HER2 phosphorylation by western blot on freshly sorted normal and IPF lung fibroblasts (FIGS. 14C and 13H). These data confirmed that the HER2 signaling was activated in SEMA7A^{high} invasive and IPF lung fibroblasts.

### Blocking ERBB2 (HER2) Blunted Fibroblast Invasion and Attenuated Pulmonary Fibrosis.

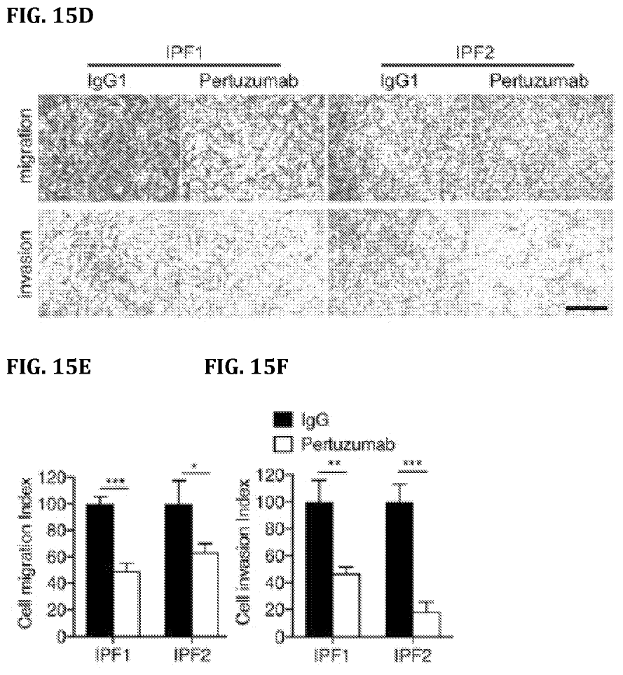

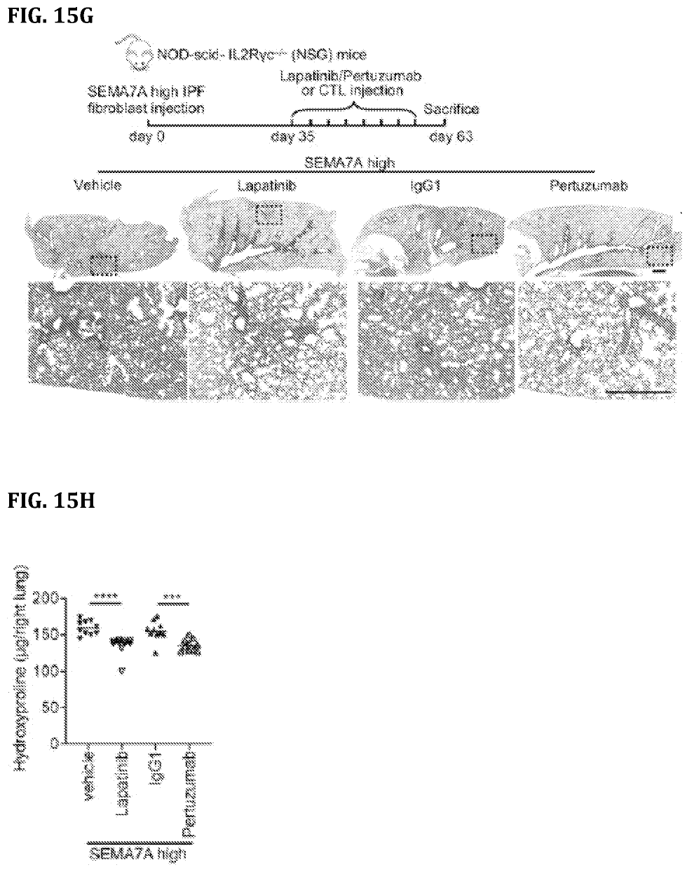

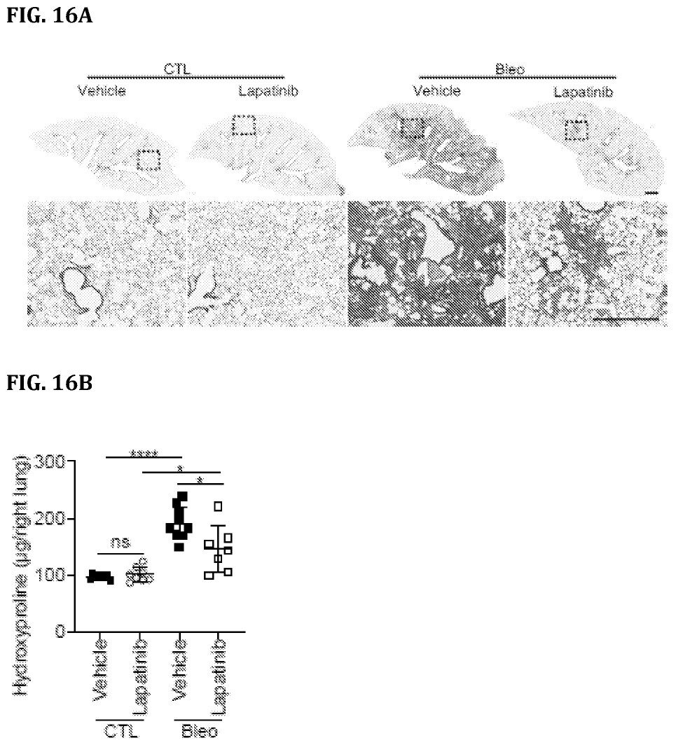

To determine if the HER2 signaling was a critical driver of lung fibroblast invasion, we blocked HER2 signaling with a HER2 inhibitor, Lapatinib, and an anti-ERBB2 monoclonal antibody, Pertuzumab (Perjeta), in migration and invasion assays. Lapatinib inhibited fibroblast migration and invasion in a dose dependent manner (FIGS. 15A-15C) and showed no effect on fibroblast viability (FIG. 14D). Pertuzumab significantly reduced fibroblast migration and invasion (FIGS. 15D-15F). We then injected SEMA7A^{high} fibroblasts from IPF patients into NSG mice and treated the mice with Lapatinib or Pertuzumab. Mice treated with Pertuzumab or Lapatinib developed significantly decreased lung fibrosis compared to the mice treated with vehicle or IgG (FIGS. 15G and 15H), which suggested that blocking ERBB2 signaling attenuated lung fibrosis. Furthermore, targeting HER2 in C57bl/6 mice remarkably reduced bleomycin-induced pulmonary fibrosis (FIGS. 16A and 16B).

### HER2 Activation Promoted Fibroblast Invasion and Fibrosis.

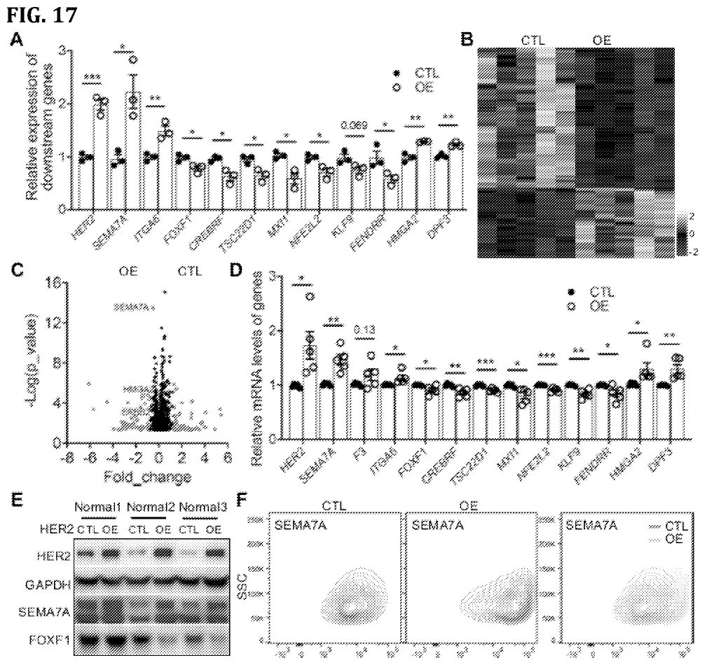

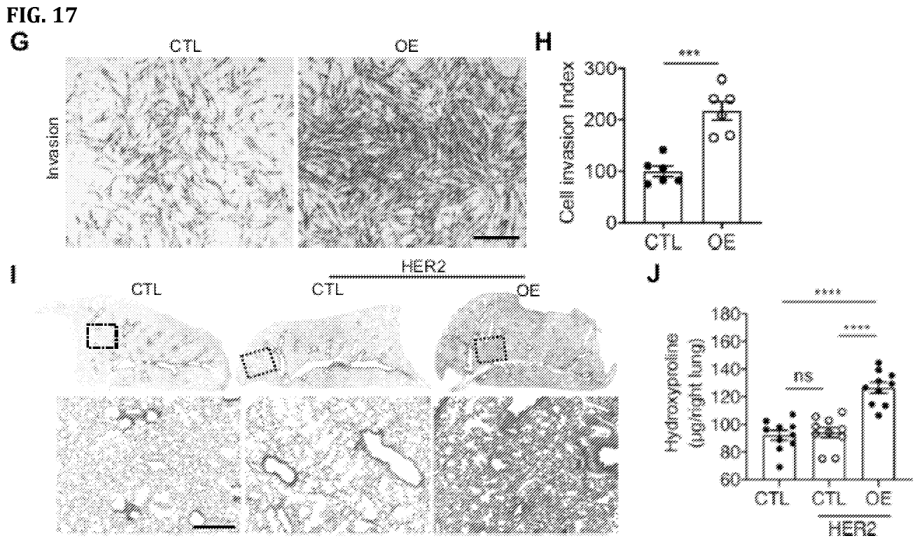

We determined if activation of HER2 is a causal factor for fibroblast invasion. To confirm this, we generated HER2 overexpression stable lines in normal human lung fibroblasts by lentivirus infection. The overexpression efficiency of HER2 was confirmed by increased total protein levels of HER2 in these stable lines (FIG. 17B). Surprisingly, the mRNA levels of invasive specific genes were elevated, while that of non-invasive specific genes were downregulated by HER2 activation in normal lung fibroblasts, confirmed by both qRT-PCR (FIG. 17A) and bulk RNA-seq (FIGS. 17B-17D). The total protein levels (FIG. 17E) and cell surface protein level (FIG. 17F) of SEMA7A, a representative invasive cell surface marker confirmed above, were also significantly increased, while FOXF1, the transcriptional factor specific to non-invasive fibroblasts, was downregulated in HER2 activated fibroblasts (FIG. 17E). These data indicated that HER2 activation activates the genetic signatures to support fibroblast invasion and subsequently increase invasion capacities of normal lung fibroblasts. The in vitro invasion assay and in vivo SCID IPF mouse model confirmed this by increased cell invasion index (FIGS. 17G and 17H) and collagen deposition (FIGS. 17I-17J) caused by the HER2 activated normal lung fibroblasts.

### HER2 Deficiency Rescued the Invasion Related Gene Signatures.

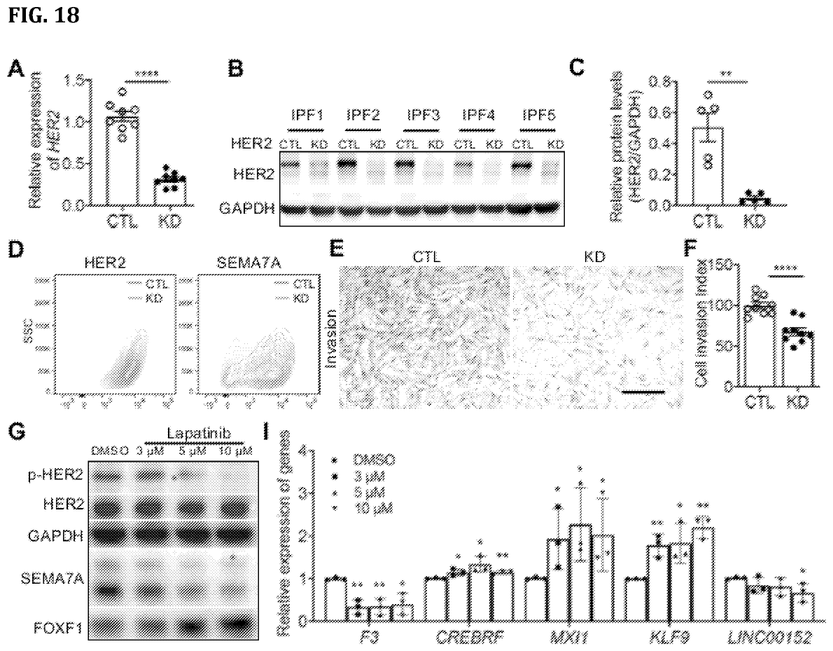

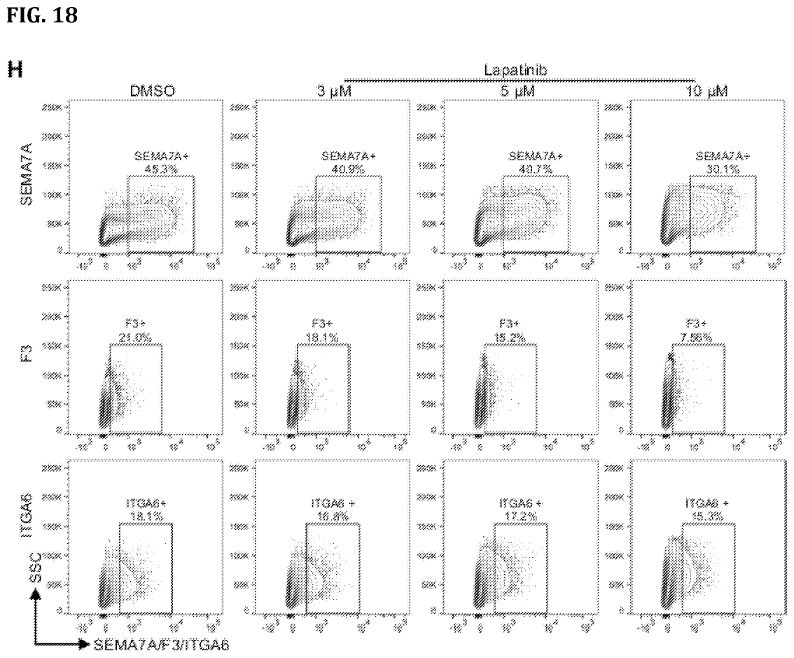

As HER2 was driving the gene programs of fibroblast invasion and mediated lung fibrosis, we then attempted to knockdown HER2 to examine the effects on IPF lung fibroblast activities. HER2 knockdown efficiency was confirmed by qRT-PCR (FIG. 18A) and Western blot (FIGS. 18B and 18C). Consistently, cell surface expression of HER2 and SEMA7A was also significantly decreased (FIG. 18D). Functionally, HER2 deficiency also caused a dramatic decrease in IPF fibroblast invasion (FIGS. 18E and 18F), confirming the crucial role of HER2 signaling in regulating fibroblast invasion.

As HER2 is a well-studied gene in cancer, several molecularly targeted therapies of HER2 positive cancers have recently become available and Lapatinib is one of most effective ones. After treating IPF lung fibroblasts with Lapatinib, decreased p-HER2 levels were detected in a dose dependent manner (FIG. 18G), showing that Lapatinib was effective to block HER2 signaling in IPF lung fibroblasts. Total protein levels of SEMA7A were decreased while FOXF1 increased with the increase of the Lapatinib concentration (FIG. 18G). We also checked the cell surface expression of SEMA7A, F3, and ITGA6, cell surface proteins specific to invasive fibroblasts, and found decreased protein levels after Lapatinib treatment (FIG. 18H). The RNA levels of the other representative genes specific to invasive or non-invasive fibroblasts were also reversed to a non-invasive state by Lapatinib treatment (FIG. 18I). These data revealed that blocking HER2 signaling by Lapatinib potentially reversed the gene programs of invasive fibroblasts and might blunt fibroblast invasion and potentially fibrosis.

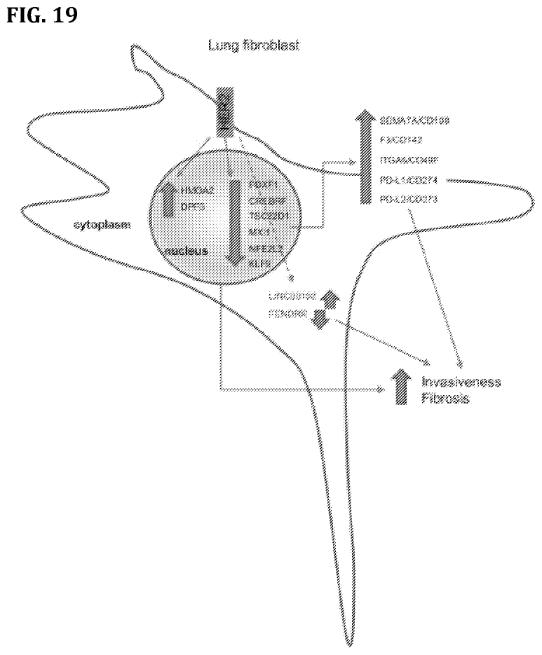

Taking together, through single cell RNA-sequencing analysis of human lung fibroblasts, we have identified invasive fibroblast clusters and non-invasive fibroblast clusters. Within these invasive fibroblast clusters, we further defined the functional roles of several cell surface markers, lncRNAs, and transcription factors. More importantly, we provide data that the HER2 signaling is the key driver of lung fibroblast invasion in IPF (FIG. 19) and serve as an attractive target for therapeutic intervention of IPF.

### Discussion

IPF, a chronic, progressive, fibrotic interstitial lung disease of unknown cause, is the most common and most lethal idiopathic interstitial pneumonia. Increasing evidence indicates that IPF is an epithelial-driven disease whereby a dysfunctional lung epithelium produces mediators of fibroblast migration, proliferation and differentiation. Activated fibroblasts secrete exaggerated amounts of ECM that subsequently remodel the lung architecture. One of the critical issues in fibroblast biology in the setting of IPF is the lack of the phenotyping-genotyping correlation of the diseased fibroblasts, which limits our understanding the pathogenesis of IPF and therapeutic development. Previous studies have shown that a subset of fibrotic fibroblasts acquire an invasive phenotype that was essential for progressive fibrogenesis. We discovered that the immune checkpoint ligand PD-L1 (CD274) was up-regulated on invasive fibroblasts and targeting PD-L1 significantly inhibited fibroblast invasion in vitro and attenuated lung fibrosis in vivo. IL11 was significantly up-regulated in the IPF lung and in invasive lung fibroblasts (FIGS. 3A and 3C). Fibroblast migration and invasion were triggered by IL11 and therapeutic targeting of IL11 prevented and reversed pulmonary fibrosis. To gain additional insights into the molecular regulation of invasive fibroblasts, we profiled invasive and non-invasive fibroblasts by single cell RNA-seq and demonstrated a classification of different clusters in invasive or non-invasive lung fibroblasts and the signature genes of each cluster. Interestingly, combining the transcription factors for IPA analysis demonstrated that the ERBB2 (HER2) signaling pathway was remarkably activated in invasive fibroblasts. Blocking HER2 inhibited fibroblast migration and invasion blunted lung fibrosis in a humanized SCID IPF model.

Several cell surface markers were identified on invasive fibroblasts. SEMA7A (Semaphorin 7A, also called CD108) is a glycosylphosphatidylinositol-anchored Semaphorin that has been previously reported to be regulated by TGF-β1 and plays a critical role in TGF-β1-induced fibrotic responses. In the present disclosure, SEMA7A was upregulated in invasive lung fibroblasts and SEMA7A^{high} fibroblasts induced more fibrogenesis than SEMA7A^{negative} fibroblasts. F3 (Coagulation Factor III, also called CD142 of tissue factor) is a cell membrane-associated protein that serves as the receptor and the essential cofactor for factors VII and VIIa. F3 is dramatically increased in lungs from patients with IPF. Similarly, in bleomycin-induced pulmonary fibrosis, the expression of F3 by alveolar macrophages, epithelial cells, and fibroblasts is dramatically enhanced. α6 (ITGA6, also called CD49f) containing integrins serve as cellular receptors for the members of the laminin family, a major structural component of the basement membrane. A recent report reveals that human IPF lung myofibroblasts express high levels of ITGA6 in vitro and in vivo, and genetic ablation of ITGA6 in collagen-expressing mesenchymal cells protects mice against bleomycin injury-induced experimental lung fibrosis. These reports are consistent with our data.

LncRNAs are recently recognized as functional regulators of tissue fibrosis and several lncRNAs are implicated in pulmonary fibrosis. LncRNA FOXF1 adjacent non-coding developmental regulatory RNA (FENDRR) dysregulation is associated with multiple types of human cancer. A recent research abstract suggested that decreased FENDRR expression may confer the altered phenotypes such as myofibroblast differentiation and cellular senescence in IPF fibroblasts, which suggested that FENDRR might be functional in lung fibrosis. In our single cell RNA-seq data, FENDRR is down-regulated in invasive fibroblasts and functional experiments reveal that FENDRR is a necessary regulator of lung fibroblast invasion, indicating that FENDRR is a functional regulator of lung fibrosis. LncRNA LINC00152 has been linked to several human cancers and promotes cell proliferation of cancer cells. However, no evidence of such activity has been reported in pulmonary fibrosis. In the present disclosure, LINC00152 is dramatically upregulated in invasive fibroblast clusters. LINC00152 knockdown in invasive fibroblasts leads to a significant decrease in IPF fibroblast invasion, showing that LINC00152 functions in pulmonary fibrosis.

Transcription factors are critical for cell transition. Based on the single cell RNA-seq data, we identified several transcription factors which show specific expression in invasive or non-invasive fibroblasts and most of which have not yet been fully delineated in lung fibrosis. FOXF1, a member of the forkhead box family of transcription factors, has previously been shown to be critical for lung development, lung regeneration after partial pneumonectomy and the inhibition of bleomycin-induced pulmonary fibrosis. TSC22D1 (TSC22 domain family, member 1) and MXI1 (MAX interactor 1) are also associated with pulmonary fibrosis in some transcriptomic, miRNomic and methylation array data. NFE2L2 (NF-E2-related transcription factor 2, also called NRF2) stimulation increases the expression of KLF9 (Kruppel-like factor 9), resulting in increases in ROS and subsequent cell death, however they show diverse functions in bleomycin-induced pulmonary fibrosis in mice and IPF. HMGA2 (High mobility group AT-hook 2) is a transcription factor that is induced by the TGF-β1/Smad3 signaling pathway and is also reported to be upregulated in pulmonary fibrosis and inhibit bleomycin-induced pulmonary fibrosis. All the above transcription factors are parts of a signaling network to regulate fibroblast invasion and are potential factors for invasive to non-invasive fibroblasts transition and are potential therapeutic targets in IPF.

HER2 (ERBB2) has been actively studied in cancer treatment for decades and was a breakthrough therapy for breast cancer. The anti-HER2 classes of drugs, such as trastuzumab, pertuzumab, lapatinib and T-DM1, for HER2-positive breast cancer have been used to treat cancer. Very limited reports have linked HER2 to pulmonary fibrosis. Targeting HER2 using 2C4, a monoclonal antibody directed against HER2 that blocks HER2/HER3 signaling, attenuated bleomycin-induced pulmonary fibrosis in mice. Reports linked EGFRs to pulmonary fibrosis by their expression levels and related genetic variation. However, cellular and molecular mechanisms of EGFRs in pulmonary fibrosis in vivo or in vitro are lacking. In the present disclosure, we combined the differentially expressed genes identified from invasive and non-invasive fibroblasts for IPA analysis and found that the HER2 signaling is dramatically upregulated in invasive fibroblasts, as well as in freshly isolated IPF fibroblasts. Blocking the HER2 signaling with pertuzumab markedly inhibited lung fibrosis in a humanized mouse fibrosis model.

The cultured fibroblasts may differ from fibroblasts in vivo in many ways. However, we do appreciate the accessibility of the cultured fibroblasts for the biological experiments, molecular manipulation in vitro, and the mechanistic studies. Importantly, these lines of investigations can be verified from fresh tissues or freshly isolated cells. Indeed, we showed that the number of SEMA7A positive fibroblasts freshly isolated from IPF lungs was higher compared to donor lungs. Higher HER2 phosphorylation was confirmed from freshly sorted IPF lung fibroblasts compared with fibroblasts from healthy donors. Although the matrigel bioassay of invasive fibroblasts may not be the perfect biologic methodology mimicking fibroblast invasion in vivo, it does provide a verifiable, experimental system to delineate fibroblast phenotype, genotype, transcription regulation, and to assess the experimental therapies. For example, with this assay we identified PD-L1 as a potential target in IPF.

In the present disclosure, we identified the HER2 signaling as a master regulator of fibroblast invasion, through regulation of an array of transcription factors. The HER2 activation also leads to upregulation of a set of cell surface molecules, enabling fibroblast adhesion and invasion, and subsequent fibrosis. The attenuation of HER2 activation effectively inhibits fibrosis in a humanized IPF model in vivo. Thus, HER2 is a novel therapeutic target for patients with IPF as described herein.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. A method for treating a subject with a progressive lung disease, the method comprising administering a pharmaceutical composition comprising a Human Epidermal Growth Factor Receptor 2 (HER2) blocking agent and a pharmaceutically acceptable carrier to the subject wherein the method improves clinical outcome compared to an untreated control.

2. The method of claim 1, wherein the progressive lung disease is pulmonary fibrosis or idiopathic pulmonary fibrosis (IPF).

3. (canceled)

4. The method of claim 1, wherein the HER2 blocking agent comprises an anti-HER2 antibody, a small molecule HER2 inhibitor, or both.

5. The method of claim 4, wherein the anti-HER2 antibody comprises one or more of trastuzumab, trastuzumab-pkrb, trastuzumab-anns, trastuzumab-dkst, trastuzumab-dttb, trastuzumab-qyyp, margutuximab-cmkb, or pertuzumab, or
wherein the anti-HER2 antibody comprises trastuzumab and pertuzumab, or
wherein the anti-HER2 antibody comprises an antibody drug conjugate.

6. (canceled)

7. The method of claim 5, wherein the anti-HER2 antibody is pertuzumab.

8. The method of claim 5, wherein the anti-HER2 antibody further comprises a recombinant hyaluronidase.

9. (canceled)

10. The method of claim 5, wherein the antibody-drug conjugate comprises trastuzumab entansine, trastuzumab deruxtecan (fam-trastuzumab deruxtecan-nxki), or disitamab vedotin.

11. The method of claim 4, wherein the small molecule HER2 inhibitor is one or more of lapatinib, neratinib, tucatanib, pyrotinib, afatinib dimaleate, dacomitinib, or a pharmaceutically acceptable salt thereof.

12. The method of claim 11, wherein the small molecule HER2 inhibitor is lapatinib.

13. The method of claim 4, wherein:
(a) the anti-HER2 antibody comprises one or more of trastuzumab, trastuzumab-pkrb, trastuzumab-anns, trastuzumab-dkst, trastuzumab-dttb, trastuzumab-qyyp, margutuximab-cmkb, or pertuzumab; and
(b) the small molecule HER2 inhibitor is one or more of lapatinib, neratinib, tucatanib, pyrotinib, afatinib dimaleate, dacomitinib, ribociclib, palbociclib, abemaciclib, or a pharmaceutically acceptable salt thereof.

14. The method of claim 1, wherein the clinical outcome is progression free survival or overall survival.

15. The method of claim 1, wherein:
(a) the method decreases one or more symptoms of the progressive lung disease; or
(b) the method increases repair of a lung injury; or
(c) the method slows progression of the progressive lung disease; or
(d) the method decreases migration, invasion, or both by lung fibroblasts obtained from the subject compared to an untreated control; or
(e) the method increases renewal of alveolar epithelial cell 2 (AEC2) cells; or
(f) a combination thereof.

16. The method of claim 1, wherein the subject is treated when one or more, two or more, three or more, four or more, or all of CD108 (SEMA7A), CD142 (F3), CD49F (ITGA6), PD-L1, and PD-L2 encoded by RNA from the subject is upregulated compared to RNA encoded by a normal healthy control subject.

17. The method of claim 16, wherein:
CD108 (SEMA7A) encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
CD142 (F3) encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
CD49F (ITGA6) encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
PD-L1 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject; and/or
PD-L2 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject.

18. The method of claim 1, wherein the subject is treated when one or more, two or more, three or more, four or more, five or more, or all of transcription factors FOXF1, CREBRF, TSC22D1, MXI1, NFE2L2, and KLF9 encoded by RNA from the subject are downregulated compared to RNA encoded by a normal healthy control subject.

19. The method of claim 18, wherein:
the transcription factor FOXF1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
the transcription factor CREBRF encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
the transcription factor TSC22D1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
the transcription factor MXI1 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject;
the transcription factor NFE2L2 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject; and/or
the transcription factor KLF9 encoded by the RNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject.

20. The method of claim 1, wherein the subject is treated when transcription factors HMGA2 and/or DPF3 encoded by RNA from the subject are upregulated compared to RNA encoded by a normal healthy control subject.

21. The method of claim 20, wherein:
the transcription factor HMGA2 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject; and/or
the transcription factor DPF3 encoded by the RNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA encoded by the normal healthy control subject.

22. The method of claim 1, wherein the subject is treated when regulation of lncRNA from the subject, which comprises one or more of LINC00152 and FENDRR, is different compared to regulation of incRNA from a normal healthy control subject such that:
the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the normal healthy control subject; and/or
the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the normal healthy control subject.

23. A method for identifying a subject with a progressive lung disease as one who can benefit therapeutically from being treated with a pharmaceutical composition comprising a Human Epidermal Growth Factor Receptor 2 (HER2) blocking agent, the method comprising:
(1) obtaining a biological sample from the subject, wherein the biological sample comprises non-invasive lung fibroblasts, invasive lung fibroblasts, or both non-invasive lung fibroblasts and invasive lung fibroblasts; and
(2) determining an RNA profile for the biological sample by:
(i) identifying and quantifying expression of RNA from the lung fibroblasts in the biological sample by RNA sequencing; and
(ii) comparing the RNA profile for the lung fibroblasts in the biological sample to an RNA profile of lung fibroblasts obtained from a normal healthy control subject,
wherein the subject is identified as the one who can benefit therapeutically from being treated with the pharmaceutical composition comprising the HER2 blocking agent, when the RNA profile from the biological sample is distinguishable from the RNA profile from the healthy control subject.

24. The method of claim 23,
wherein the subject is identified as the one who can benefit therapeutically from being treated with the pharmaceutical composition comprising the HER2 blocking agent, when:
CD108 (SEMA7A) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to RNA from lung fibroblasts of the normal healthy control subject,
CD142 (F3) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
CD49F (ITGA6) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
PD-L1 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject, and/or
PD-L2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject; or
wherein the subject is identified as the one who can benefit therapeutically from being treated with the pharmaceutical composition comprising the HER2 blocking agent; when:
transcription factor FOXF1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
transcription factor CREBRF encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
transcription factor TSC22D1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA front lung fibroblasts of the normal healthy control subject,
transcription factor MXI1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
transcription factor NFE2L2 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject, and/or
transcription factor KLF9 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject; or
wherein the subject is identified as the one who can benefit therapeutically from being treated with the pharmaceutical composition comprising the HER2 blocking agent, when:
transcription factor HMGA2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject, and/or
transcription factor DPF3 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject; or
wherein the subject is identified as the one who can benefit therapeutically from being treated with the pharmaceutical composition comprising the HER2 blocking agent, when regulation of lncRNA from the subject, which comprises one or more of LINC00152 and FENDRR, is different compared to regulation of lncRNA from the normal healthy control subject such that:
the LINC00152 encoded by the lncRNA front the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the normal healthy control subject, and/or
the FENDRR encoded by the incRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the normal healthy control subject.

25-27. (canceled)

28. A method for treating a subject with a progressive lung disease and identified as one who can benefit therapeutically from being treated with a pharmaceutical composition comprising a Human Epidermal Growth Factor Receptor 2 (HER2) blocking agent, the method comprising treating the identified subject with the pharmaceutical composition comprising the HER2 blocking agent such that clinical outcome improves by the treating,
wherein the subject is identified as the one who can benefit therapeutically from being treated with the pharmaceutical composition by:
(1) obtaining or having obtained a biological sample from the subject, wherein the biological sample comprises non-invasive lung fibroblasts, invasive lung fibroblasts, or both non-invasive lung fibroblasts and invasive lung fibroblasts; and
(2) determining or having determined an RNA profile for the biological sample by:
(i) identifying and quantifying or having identified and quantified expression of RNA from the lung fibroblasts in the biological sample by RNA sequencing; and
(ii) comparing or having compared the RNA profile for the lung fibroblasts in the biological sample to an RNA profile of lung fibroblasts obtained from a healthy control subject, and
wherein the subject is identified as the one who can benefit therapeutically from being treated with the pharmaceutical composition comprising the HER2 blocking agent, when the RNA profile from the biological sample is distinguishable from the RNA profile from the healthy control subject, or
wherein the subject is identified as the one who can benefit therapeutically from being treated with the pharmaceutical composition comprising the HER2 blocking agent, when:
CD108 (SEMA7) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to RNA from lung fibroblasts of the normal healthy control subject,
CD142 (F3 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
CD49F (ITGA6) encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold contoured to the RNA from lung fibroblasts of the normal healthy control subject,
PD-L1 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from hug fibroblasts of the normal healthy control subject, and/or
PD-L2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1 fold to about 5 fold, about 1.5 fold to about 3 fold, or about 2.5 fold to about 5 fold compared to the RNA from long fibroblasts of the normal healthy control subject; or
wherein the subject is identified as the one who can benefit therapeutically from being treated with the pharmaceutical composition comprising the HER2 blocking agent, when:
transcription facto FOXF1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
transcription factor CREBRF encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject,
transcription factor TSC22D1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 Nod to about 5 fold, about 1.5 fold to about 3 fold, or about 2.5 fold to about 5 fold compared to the RNA from lung fibroblasts of the normal healthy control subject,
transcription factor MXI1 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold t, about 5 fold compared to the RNA from lung fibroblasts of the normal healthy control subject,
transcription factor NFE2L2 encoded by the RNA front the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject, and/or
transcription actor KLF9 encoded by the RNA from the lung fibroblasts in the biological sample is downregulated about 1.5 fold, about 2 fold, about 2.5 fold, about 3 fold, about 3.5 fold, about 4 fold, about 4.5 fold, about 5 fold, about 1.5 fold to abut 5 fold, about 1.5 fold to about 3 fold, or about 2.5 fold to about 5 fold compared to the RNA from lung fibroblasts of the normal healthy control subject; or
wherein the subject is identified as the one who can benefit therapeutically from being treated with the pharmaceutical composition comprising the HER2 blocking agent, when:
transcription factor HMGA2 encoded by the RNA from the lung fibroblasts in the biological sample is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the RNA from lung fibroblasts of the normal healthy control subject, and/or
transcription factor DPF3 encoded by the RNA from the lung fibroblasts in the biological sample is upregulated about 1.5 fold, about 2 fold, about 2.5 fold, about 3 fold, about 3.5 fold, about 4 fold, about 4 5 fold, about 5 fold, about 1.5 fold to about 5 fold, about 1.5 fold to about 3 fold, or about 2.5 fold to about 5 fold compared to the RNA from lung fibroblasts of the normal healthy control subject; or
wherein the subject is identified as the one who can benefit therapeutically from being treated with the pharmaceutical composition comprising the HER2 blocking agent, when regulation of incRNA from the subject, which comprises one or more of LINC00152 and FENDRR is different compared to regulation of lncRNA from the normal healthy control subject such that:
the LINC00152 encoded by the lncRNA from the subject is upregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the normal healthy control subject, and/or
the FENDRR encoded by the lncRNA from the subject is downregulated about 1.5 fold, about 2 fold, about 2.5 fold, about 3 fold, about 3.5 fold, about 4 fold, about 4.5 fold, about 5 fold, about 1.5 fold to about 5 fold, about 1.5 fold to about 3 fold, or about 2.5 fold to about 5 fold compared to the normal healthy control subject.

29-32. (canceled)


SEQUENCE LISTING (28 sequences)

SEQ ID NO: 1 - DNA, 20 bp, Homo sapiens
tgtgtattccctcggtgaca

SEQ ID NO: 2 - DNA, 20 bp, Homo sapiens
gatctccatcatgttgaagg

SEQ ID NO: 3 - DNA, 23 bp, Homo sapiens
tccccagagttcacaccttacct

SEQ ID NO: 4 - DNA, 22 bp, Homo sapiens
cacttttgttcccacctgttca

SEQ ID NO: 5 - DNA, 21 bp, Homo sapiens
gagcttttgtgatgggcgatt

SEQ ID NO: 6 - DNA, 22 bp, Homo sapiens
ctctccaccaacttcataaggc

SEQ ID NO: 7 - DNA, 20 bp, Homo sapiens
aattgctgggctgctttcta

SEQ ID NO: 8 - DNA, 20 bp, Homo sapiens
ttcacaatggctcagtgctc

SEQ ID NO: 9 - DNA, 21 bp, Homo sapiens
tcttcacagcacagttcctgg

SEQ ID NO: 10 - DNA, 20 bp, Homo sapiens
ggctgagtcgtgattttcgg

SEQ ID NO: 11 - DNA, 19 bp, Homo sapiens
gcggcttccgaaggaaatg

SEQ ID NO: 12 - DNA, 20 bp, Homo sapiens
caagtggccgttcatcatgc

SEQ ID NO: 13 - DNA, 21 bp, Homo sapiens
gccatctgagtggaatcgaga

SEQ ID NO: 14 - DNA, 23 bp, Homo sapiens
ccgtgacttcttaactgcgtatt

SEQ ID NO: 15 - DNA, 21 bp, Homo sapiens
ctgacgacacccctggtggat

SEQ ID NO: 16 - DNA, 27 bp, Homo sapiens
cgattttgttgtcaatagctaccacac

SEQ ID NO: 17 - DNA, 24 bp, Homo sapiens
caacgtgcagcgtctgctggaggc

SEQ ID NO: 18 - DNA, 23 bp, Homo sapiens
cgattcttttccagctcattgtg

SEQ ID NO: 19 - DNA, 21 bp, Homo sapiens
tggctgtgggaaagtctatgg

SEQ ID NO: 20 - DNA, 20 bp, Homo sapiens
ctcgtctgagcgggagaact

SEQ ID NO: 21 - DNA, 19 bp, Homo sapiens
agcccagcacatccagtca

SEQ ID NO: 22 - DNA, 25 bp, Homo sapiens
tgcatgcagtcatcaaagtacaaag

SEQ ID NO: 23 - DNA, 21 bp, Homo sapiens
aaagcagctcaaaagaaagca

SEQ ID NO: 24 - DNA, 21 bp, Homo sapiens
tgttgtggccatttcctaggt

SEQ ID NO: 25 - DNA, 22 bp, Homo sapiens
ggctgctggagataaaacctga

SEQ ID NO: 26 - DNA, 21 bp, Homo sapiens
ttcctggatgctttcctcctc

SEQ ID NO: 27 - DNA, 21 bp, Artificial Sequence
  synthetic
ccagccaugugauuccaaatt

SEQ ID NO: 28 - DNA, 21 bp, Artificial Sequence
  synthetic
gcgauugacugucuuauaatt